(19) United States
(12) Patent Application Publication
(10) Pub. No.: US 2021/0107961 A1
(43) Pub. Date: Apr. 15, 2021

(54) Fusion Protein Constructs Comprising an Anti-MUC1 Antibody and IL-15

(71) Applicant: Glycotope GmbH, Berlin (DE)

(72) Inventors: Johanna GELLERT, Berlin (DE); Patrik KEHLER, Berlin (DE); Anika JÄKEL, Berlin (DE); Laura DIX, Berlin (DE); Antje DANIELCZYK, Berlin (DE); Steffen GOLETZ, Berlin (DE)

(21) Appl. No.: 16/971,756

(22) PCT Filed: Mar. 1, 2019
(86) PCT No.: PCT/EP2019/055125

§ 371 Date: Aug. 21, 2020

(30) Foreign Application Priority Data
Mar. 1, 2018 (EP) 18159449.0
Sep. 13, 2018 (EP) 18194290.5

(51) Int. Cl. C07K 14/54; C07K 16/30; A61P 35/00

(52) U.S. Cl. CPC C07K 14/5443; C07K 16/3092; A61P 35/00; C07K 2319/00; A61K 2039/505; C07K 2317/24; C07K 2317/732; C07K 2317/90; C07K 2317/41

(57) ABSTRACT

The present invention pertains to fusion protein constructs comprising an antibody against the cancer antigen MUC1 and IL-15. In particular, the fusion protein constructs activate NK cells and T cells at the cancer site.

### FIELD OF THE INVENTION

The present invention pertains to the field of antibodies. A fusion protein of an antibody against a cancer antigen and a cytokine is provided. In particular, the fusion protein activates T cells and natural killer cells—via its IL-15 part—at the cancer site—by binding to the cancer antigen MUC1. The design of the fusion protein constructs shows distinct advantages in the specific setting provided, in particular highly specific targeting of the tumor sites with strong antigen binding and high immune cell activation. In specific embodiments, the present invention is directed to the therapeutic and diagnostic use of these fusion protein constructs.

### BACKGROUND OF THE INVENTION

Cytokines are promising drugs for anti-cancer treatment as they modulate immune responses. Cytokines are molecular messengers that allow the cells of the immune system to communicate with one another to generate a coordinated response to a target antigen. While many forms of communication of the immune system occur through direct cell-cell interaction, the secretion of cytokines enables the rapid propagation of immune signaling in a multifaceted and efficient manner.

Cytokines directly stimulate immune effector cells and stromal cells at the tumor site and enhance tumor cell recognition by cytotoxic effector cells. Numerous animal tumor model studies have demonstrated that cytokines have broad anti-tumor activity and this has been translated into a number of cytokine-based approaches for cancer therapy.

Recent years have seen a number of cytokines, including GM-CSF, IL-7, IL-12, IL-15, IL-18 and IL-21, enter clinical trials for patients with advanced cancer. There is ongoing pre-clinical work supporting the neutralization of suppressive cytokines, such as IL-10 and TGF-β in promoting anti-tumor immunity.

Particularly interleukin-15 (IL-15) is an attractive cytokine for cancer therapy. IL-15 is a cytokine that stimulates effector immune responses. It induces development, activation and proliferation of T cells and natural killer (NK) cells. IL-15 and its IL-15 receptor α-chain are expressed on monocytes, macrophages and dendritic cells and bind to the IL-2 receptor β- common γ-chain (IL2Rβγ_{c}) complex on effector immune cells. IL-15 induces high levels of anti-tumor cytotoxicity when used in combination with common tumor targeting antibodies in vitro and in vivo. Yet, administration of cytokines is often hindered by dose-limiting toxicities preventing their use as effective modulators.

In view of this, there is a need in the art for effective targeting of cytokines such as IL-15 to the tumor site. As a prerequisite for safe and effective immunocytokine therapy highly specific tumor targeting is necessary.

### SUMMARY OF THE INVENTION

The present inventors have found interleukin 15 can effectively and specifically be targeted to tumor sites by combining it with anti-MUC1 antibodies. TA-MUC1 is a novel carbohydrate/protein mixed epitope on the tumor marker MUC1 that is virtually absent from normal cells. TA-MUC1 shows a broad distribution among epithelial cancers of different origin and is also present on metastases and cancer stem cells underpinning its broad therapeutic potential. Simultaneous binding of the anti-cancer antigen MUC1 and IL2Rβγ_{c} enables activation and proliferation of NK and T cells directly at the tumor site. The present inventors could now proof that a fusion protein comprising an antibody specific for MUC1 and IL-15 effectively targets cancer cells and induces a NK and T cell response against said cancer cells.

Therefore, in a first aspect, the present invention is directed to a fusion protein construct, comprising
(i) an antibody module specifically binding to MUC1, and
(ii) an IL-15 module.

In a second aspect, the present invention provides a nucleic acid encoding the fusion protein construct according to the invention. Furthermore, in a third aspect an expression cassette or vector comprising the nucleic acid according to the invention and a promoter operatively connected with said nucleic acid and, in a fourth aspect, a host cell comprising the nucleic acid or the expression cassette or vector according to the invention are provided.

In a fifth aspect, the present invention is directed to a pharmaceutical composition comprising the fusion protein construct according to the invention.

According to a sixth aspect, the invention provides the fusion protein construct or the pharmaceutical composition according to the invention for use in medicine, in particular in the treatment of cancer or infections.

Other objects, features, advantages and aspects of the present invention will become apparent to those skilled in the art from the following description and appended claims. It should be understood, however, that the following description, appended claims, and specific examples, which indicate preferred embodiments of the application, are given by way of illustration only. Various changes and modifications within the spirit and scope of the disclosed invention will become readily apparent to those skilled in the art from reading the following.

### Definitions

As used herein, the following expressions are generally intended to preferably have the meanings as set forth below, except to the extent that the context in which they are used indicates otherwise.

The expression “comprise”, as used herein, besides its literal meaning also includes and specifically refers to the expressions “consist essentially of” and “consist of”. Thus, the expression “comprise” refers to embodiments wherein the subject-matter which “comprises” specifically listed elements does not comprise further elements as well as embodiments wherein the subject-matter which “comprises” specifically listed elements may and/or indeed does encompass further elements. Likewise, the expression “have” is to be understood as the expression “comprise”, also including and specifically referring to the expressions “consist essentially of” and “consist of”. The term “consist essentially of”, where possible, in particular refers to embodiments wherein the subject-matter comprises 20% or less, in particular 15% or less, 10% or less or especially 5% or less further elements in addition to the specifically listed elements of which the subject-matter consists essentially of.

As used herein, the term “protein” refers to a polypeptide or a combination of two or more polypeptides or a complex comprising one or more polypeptides and one or more other molecules or ions. A protein can contain any of the naturally occurring amino acids as well as artificial amino acids and can be of biologic or synthetic origin. The polypeptide(s) of a protein may be modified, naturally (post-translational modifications) or synthetically, by e.g. glycosylation, amidation, carboxylation, hydroxylation and/or phosphorylation. A polypeptide comprises at least two amino acids, but does not have to be of any specific length; this term does not include any size restrictions. Preferably, a polypeptide comprises at least 50 amino acids, more preferably at least 100 amino acids, most preferably at least 150 amino acids.

The term “fusion protein construct” refers to a protein wherein two or more polypeptides derived from different naturally occurring proteins are artificially combined to form one protein. The different polypeptides may in particular be fused to each other so as to form one polypeptide chain comprising said different polypeptides.

The terms “protein” and “protein construct”, as used herein, refer in certain embodiments to a population of proteins or protein constructs, respectively, of the same kind. In particular, all proteins or protein constructs of the population exhibit the features used for defining the protein or protein construct. In certain embodiments, all proteins or protein constructs in the population have the same amino acid sequence.

The term “antibody” in particular refers to a protein comprising at least two heavy chains and two light chains connected by disulfide bonds. Each heavy chain is comprised of a heavy chain variable region (VH) and a heavy chain constant region (CH). Each light chain is comprised of a light chain variable region (VL) and a light chain constant region (CL). The heavy chain-constant region comprises three or—in the case of antibodies of the IgM- or IgE-type—four heavy chain-constant domains (CH1, CH2, CH3 and CH4) wherein the first constant domain CH1 is adjacent to the variable region and may be connected to the second constant domain CH2 by a hinge region. The light chain-constant region consists only of one constant domain. The variable regions can be further subdivided into regions of hypervariability, termed complementarity determining regions (CDRs), interspersed with regions that are more conserved, termed framework regions (FR), wherein each variable region comprises three CDRs and four FRs. The variable regions of the heavy and light chains contain a binding domain that interacts with an antigen. The heavy chain constant regions may be of any type such as γ-, δ-, α-, μ- or ε-type heavy chains. Preferably, the heavy chain of the antibody is a γ-chain. Furthermore, the light chain constant region may also be of any type such as κ- or λ-type light chains. Preferably, the light chain of the antibody is a κ-chain. The constant regions of the antibodies may mediate the binding of the immunoglobulin to host tissues or factors, including various cells of the immune system (e.g., effector cells) and the first component (C1q) of the classical complement system. The antibody can be e.g. a humanized, human or chimeric antibody.

The antigen-binding portion of an antibody usually refers to full length or one or more fragments of an antibody that retains the ability to specifically bind to an antigen. It has been shown that the antigen-binding function of an antibody can be performed by fragments of a full-length antibody. Examples of binding fragments of an antibody include a Fab fragment, a monovalent fragment consisting of the V_{L}, V_{H}, C_{L} and C_{H1} domains; a F(ab)₂ fragment, a bivalent fragment comprising two Fab fragments, each of which binds to the same antigen, linked by a disulfide bridge at the hinge region; a Fd fragment consisting of the V_{H} and C_{H1} domains; a Fv fragment consisting of the V_{L} and V_{H} domains of a single arm of an antibody; and a dAb fragment, which consists of a V_{H} domain.

The “Fab part” of an antibody in particular refers to a part of the antibody comprising the heavy and light chain variable regions (V_{H} and V_{L}) and the first domains of the heavy and light chain constant regions (C_{H1} and C_{L}). In cases where the antibody does not comprise all of these regions, then the term “Fab part” only refers to those of the regions V_{H}, V_{L}, C_{H1} and C_{L} which are present in the antibody. Preferably, “Fab part” refers to that part of an antibody corresponding to the fragment obtained by digesting a natural antibody with papain which contains the antigen binding activity of the antibody.

In particular, the Fab part of an antibody encompasses the antigen binding site or antigen binding ability thereof. Preferably, the Fab part comprises at least the V_{H} region of the antibody.

The “Fc part” of an antibody in particular refers to a part of the antibody comprising the heavy chain constant regions 2, 3 and—where applicable—4 (C_{H2}, C_{H3} and C_{H4}). In particular, the Fc part comprises two of each of these regions. In cases where the antibody does not comprise all of these regions, then the term “Fc part” only refers to those of the regions C_{H2}, C_{H3} and C_{H4} which are present in the antibody. Preferably, the Fc part comprises at least the C_{H2} region of the antibody. Preferably, “Fc part” refers to that part of an antibody corresponding to the fragment obtained by digesting a natural antibody with papain which does not contain the antigen binding activity of the antibody. In particular, the Fc part of an antibody is capable of binding to the Fc receptor and thus, e.g. comprises an Fc receptor binding site or an Fc receptor binding ability.

The terms “antibody” and “antibody construct”, as used herein, refer in certain embodiments to a population of antibodies or antibody constructs, respectively, of the same kind. In particular, all antibodies or antibody constructs of the population exhibit the features used for defining the antibody or antibody construct. In certain embodiments, all antibodies or antibody constructs in the population have the same amino acid sequence.

The term “antibody” as used herein also includes fragments and derivatives of said antibody. A “fragment or derivative” of an antibody in particular is a protein or glycoprotein which is derived from said antibody and is capable of binding to the same antigen, in particular to the same epitope as the antibody. Thus, a fragment or derivative of an antibody herein generally refers to a functional fragment or derivative. In particularly preferred embodiments, the fragment or derivative of an antibody comprises a heavy chain variable region. It has been shown that the antigen-binding function of an antibody can be performed by fragments of a full-length antibody or derivatives thereof. Examples of fragments of an antibody include (i) Fab fragments, monovalent fragments consisting of the variable region and the first constant domain of each the heavy and the light chain; (ii) F(ab)₂ fragments, bivalent fragments comprising two Fab fragments linked by a disulfide bridge at the hinge region; (iii) Fd fragments consisting of the variable region and the first constant domain CH1 of the heavy chain; (iv) Fv fragments consisting of the heavy chain and light chain variable region of a single arm of an antibody; (v) scFv fragments, Fv fragments consisting of a single polypeptide chain; (vi) (Fv)₂ fragments consisting of two Fv fragments covalently linked together; (vii) a heavy chain variable domain; and (viii) multibodies consisting of a heavy chain variable region and a light chain variable region covalently linked together in such a manner that association of the heavy chain and light chain variable regions can only occur intermolecular but not intramolecular. Derivatives of an antibody in particular include antibodies which bind to the same antigen as the parent antibody, but which have a different amino acid sequence than the parent antibody from which it is derived. These antibody fragments and derivatives are obtained using conventional techniques known to those with skill in the art.

A target amino acid sequence is “derived” from or “corresponds” to a reference amino acid sequence if the target amino acid sequence shares a homology or identity over its entire length with a corresponding part of the reference amino acid sequence of at least 75%, more preferably at least 80%, at least 85%, at least 90%, at least 93%, at least 95%, at least 97%, at least 98% or at least 99%. The “corresponding part” means that, for example, framework region 1 of a heavy chain variable region (FRH1) of a target antibody corresponds to framework region 1 of the heavy chain variable region of the reference antibody. In particular embodiments, a target amino acid sequence which is “derived” from or “corresponds” to a reference amino acid sequence is 100% homologous, or in particular 100% identical, over its entire length with a corresponding part of the reference amino acid sequence. A “homology” or “identity” of an amino acid sequence or nucleotide sequence is preferably determined according to the invention over the entire length of the reference sequence or over the entire length of the corresponding part of the reference sequence which corresponds to the sequence which homology or identity is defined. An antibody derived from a parent antibody which is defined by one or more amino acid sequences, such as specific CDR sequences or specific variable region sequences, in particular is an antibody having amino acid sequences, such as CDR sequences or variable region sequences, which are at least 75%, preferably at least 80%, at least 85%, at least 90%, at least 93%, at least 95%, at least 97%, at least 98% or at least 99% homologous or identical, especially identical, to the respective amino acid sequences of the parent antibody. In certain embodiments, the antibody derived from (i.e. derivative of) a parent antibody comprises the same CDR sequences as the parent antibody, but differs in the remaining sequences of the variable regions.

The term “antibody” as used herein also refers to multivalent and multispecific antibodies, i.e. antibody constructs which have more than two binding sites each binding to the same epitope and antibody constructs which have one or more binding sites binding to a first epitope and one or more binding sites binding to a second epitope, and optionally even further binding sites binding to further epitopes.

“Specific binding” preferably means that an agent such as an antibody binds stronger to a target such as an epitope for which it is specific compared to the binding to another target. An agent binds stronger to a first target compared to a second target if it binds to the first target with a dissociation constant (K_{d}) which is lower than the dissociation constant for the second target. Preferably the dissociation constant for the target to which the agent binds specifically is more than 100-fold, 200-fold, 500-fold or more than 1000-fold lower than the dissociation constant for the target to which the agent does not bind specifically. Furthermore, the term “specific binding” in particular indicates a binding affinity between the binding partners with an affinity constant Kₐ of at least 10⁶ M⁻¹, preferably at least 10⁷ M⁻¹, more preferably at least 10⁸ M⁻¹. An antibody specific for a certain antigen in particular refers to an antibody which is capable of binding to said antigen with an affinity having a Kₐ of at least 10⁶ M⁻¹, preferably at least 10⁷ M⁻¹, more preferably at least 10⁸ M⁻¹. For example, the term “anti-MUC1 antibody” in particular refers to an antibody specifically binding MUC1 and preferably is capable of binding to MUC1 with an affinity having a Kₐ of at least 10⁶ M⁻¹, preferably at least 10⁷ M⁻¹, more preferably at least 10⁸ M⁻¹.

An “antibody module” as referred to herein refers to a polypeptide construct which is derived from an antibody and is capable of specifically binding to an antigen. In particular, the antibody module comprises at least one, especially two, antibody heavy chains and optionally at least one, especially two, antibody light chains.

The term “antigen binding fragment” as used herein refers to a polypeptide construct which is derived from an antibody, is capable of specifically binding to an antigen, but does not comprise all elements of a natural antibody. In particular, the antigen binding fragment does not comprise some or all of the constant domains of an antibody, and may comprise only one instead of two antigen binding sites.

An “antigen binding site” in particular comprises at least one antibody variable region, for example an antibody heavy chain variable region. In specific embodiments, an antigen binding site comprises an antibody heavy chain variable region and an antibody light chain variable region. The antibody heavy chain variable region and the antibody light chain variable region of an antigen binding site may be arranged to each other using an antigen scaffold, in particular a CH1 domain and a CL domain, and/or may be fused to each other via a peptide linker. In certain embodiments, an antigen binding site is a single chain variable region fragment (scFv).

“IL-15” refers to the cytokine interleukin 15, in particular to human interleukin 15. IL-15 is a four α-helix bundle protein which is expressed with an N terminal signal peptide. In the mature IL-15, the signal peptide is cleaved off and the protein is glycosylated, having a mass of about 14-15 kDa. IL-15 binds to the IL-15 receptor α chain, in particular to the sushi domain thereof, and to a complex of the IL-2 receptor β-chain and the common interleukin receptor γ-chain (common γ-chain).

The term “MUC1” refers to the protein MUC1, also known as mucin-1, polymorphic epithelial mucin (PEM) or cancer antigen 15-3, in particular to human MUC1. MUC1 is a member of the mucin family and encodes a membrane bound, glycosylated phosphoprotein. MUC1 has a core protein mass of 120-225 kDa which increases to 250-500 kDa with glycosylation. It extends 200-500 nm beyond the surface of the cell. The protein is anchored to the apical surface of many epithelial cells by a transmembrane domain. The extracellular domain includes a 20 amino acid variable number tandem repeat (VNTR) domain, with the number of repeats varying from 20 to 120 in different individuals. These repeats are rich in serine, threonine and proline residues which permits heavy 0-glycosylation. In certain embodiments, the term “MUC1” refers to tumor-associated MUC1 (“TA-MUC1”). TA-MUC1 is MUC1 present on cancer cells. This MUC1 differs from MUC1 present on non-cancer cells in its much higher expression level, its localization and its glycosylation. In particular, TA-MUC1 is present apolarly over the whole cell surface in cancer cells, while in non-cancer cells MUC1 has a strictly apical expression and hence, is not accessible for systemically administered antibodies. Furthermore, TA-MUC1 has an aberrant 0-glycosylation which exposes new peptide epitopes on the MUC1 protein backbone and new carbohydrate tumor antigens such as the Thomsen-Friedenreich antigen alpha (TFα).

“TFα”, also called Thomsen-Friedenreich antigen alpha or Core-1, refers to the disaccharide Gal-β1,3-GaINAc which is O-glycosidically linked in an alpha-anomeric configuration to the hydroxy amino acids serine or threonine of proteins in carcinoma cells.

A “relative amount of glycans” according to the invention refers to a specific percentage or percentage range of the glycans attached to the antibodies of an antibody preparation or in a composition comprising antibodies, respectively. In particular, the relative amount of glycans refers to a specific percentage or percentage range of all glycans comprised in the antibodies and thus, attached to the polypeptide chains of the antibodies in an antibody preparation or in a composition comprising antibodies. 100% of the glycans refers to all glycans attached to the antibodies of the antibody preparation or in a composition comprising antibodies, respectively. For example, a relative amount of glycans carrying fucose of 10% refers to a composition comprising antibodies wherein 10% of all glycans comprised in the antibodies and thus, attached to the antibody polypeptide chains in said composition comprise a fucose residue while 90% of all glycans comprised in the antibodies and thus, attached to the antibody polypeptide chains in said composition do not comprise a fucose residue. The corresponding reference amount of glycans representing 100% may either be all glycan structures attached to the antibodies in the composition, or all N-glycans, i.e. all glycan structures attached to an asparagine residue of the antibodies in the composition, or all complex-type glycans. The reference group of glycan structures generally is explicitly indicated or directly derivable from the circumstances by the skilled person.

The term “N-glycosylation” refers to all glycans attached to asparagine residues of the polypeptide chain of a protein. These asparagine residues generally are part of N-glycosylation sites having the amino acid sequence Asn-Xaa-Ser/Thr, wherein Xaa may be any amino acid except for proline. Likewise, “N-glycans” are glycans attached to asparagine residues of a polypeptide chain. The terms “glycan”, “glycan structure”, “carbohydrate”, “carbohydrate chain” and “carbohydrate structure” are generally used synonymously herein. N-glycans generally have a common core structure consisting of two N-acetylglucosamine (GlcNAc) residues and three mannose residues, having the structure Mana1,6-(Manα1,3-)Manβ1,4-GIcNAcβ1,4-GIcNAcβ1-Asn with Asn being the asparagine residue of the polypeptide chain. N-glycans are subdivided into three different types, namely complex-type glycans, hybrid-type glycans and high mannose-type glycans.

The numbers given herein, in particular the relative amounts of a specific glycosylation property, are preferably to be understood as approximate numbers. In particular, the numbers preferably may be up to 10% higher and/or lower, in particular up to 9%, 8%, 7%, 6%, 5%,4%, 3%, 2% or 1% higher and/or lower.

The term “nucleic acid” includes single-stranded and double-stranded nucleic acids and ribonucleic acids as well as deoxyribonucleic acids. It may comprise naturally occurring as well as synthetic nucleotides and can be naturally or synthetically modified, for example by methylation, 5′- and/or 3′-capping.

The term “expression cassette” in particular refers to a nucleic acid construct which is capable of enabling and regulating the expression of a coding nucleic acid sequence introduced therein. An expression cassette may comprise promoters, ribosome binding sites, enhancers and other control elements which regulate transcription of a gene or translation of an mRNA. The exact structure of expression cassette may vary as a function of the species or cell type, but generally comprises 5′-untranscribed and 5′- and 3′-untranslated sequences which are involved in initiation of transcription and translation, respectively, such as TATA box, capping sequence, CAAT sequence, and the like. More specifically, 5′-untranscribed expression control sequences comprise a promoter region which includes a promoter sequence for transcriptional control of the operatively connected nucleic acid. Expression cassettes may also comprise enhancer sequences or upstream activator sequences.

According to the invention, the term “promoter” refers to a nucleic acid sequence which is located upstream (5′) of the nucleic acid sequence which is to be expressed and controls expression of the sequence by providing a recognition and binding site for RNA-polymerases. The “promoter” may include further recognition and binding sites for further factors which are involved in the regulation of transcription of a gene. A promoter may control the transcription of a prokaryotic or eukaryotic gene. Furthermore, a promoter may be “inducible”, i.e. initiate transcription in response to an inducing agent, or may be “constitutive” if transcription is not controlled by an inducing agent. A gene which is under the control of an inducible promoter is not expressed or only expressed to a small extent if an inducing agent is absent. In the presence of the inducing agent the gene is switched on or the level of transcription is increased. This is mediated, in general, by binding of a specific transcription factor.

The term “vector” is used here in its most general meaning and comprises any intermediary vehicle for a nucleic acid which enables said nucleic acid, for example, to be introduced into prokaryotic and/or eukaryotic cells and, where appropriate, to be integrated into a genome. Vectors of this kind are preferably replicated and/or expressed in the cells. Vectors comprise plasmids, phagemids, bacteriophages or viral genomes. The term “plasmid” as used herein generally relates to a construct of extrachromosomal genetic material, usually a circular DNA duplex, which can replicate independently of chromosomal DNA.

According to the invention, the term “host cell” relates to any cell which can be transformed or transfected with an exogenous nucleic acid. The term “host cells” comprises according to the invention prokaryotic (e.g. E. coli) or eukaryotic cells (e.g. mammalian cells, in particular human cells, yeast cells and insect cells). Particular preference is given to mammalian cells such as cells from humans, mice, hamsters, pigs, goats, or primates. The cells may be derived from a multiplicity of tissue types and comprise primary cells and cell lines. A nucleic acid may be present in the host cell in the form of a single copy or of two or more copies and, in one embodiment, is expressed in the host cell.

The term “patient” means according to the invention a human being, a nonhuman primate or another animal, in particular a mammal such as a cow, horse, pig, sheep, goat, dog, cat or a rodent such as a mouse and rat. In a particularly preferred embodiment, the patient is a human being.

The term “cancer” according to the invention in particular comprises leukemias, seminomas, melanomas, carcinomas, teratomas, lymphomas, sarcomas, mesotheliomas, neuroblastomas, gliomas, rectal cancer, endometrial cancer, kidney cancer, adrenal cancer, thyroid cancer, blood cancer, skin cancer, cancer of the brain, cervical cancer, intestinal cancer, liver cancer, colon cancer, stomach cancer, intestine cancer, head and neck cancer, gastrointestinal cancer, lymph node cancer, esophagus cancer, colorectal cancer, pancreas cancer, ear, nose and throat (ENT) cancer, breast cancer, prostate cancer, bladder cancer, cancer of the uterus, ovarian cancer and lung cancer and the metastases thereof. The term cancer according to the invention also comprises cancer metastases.

By “tumor” is meant a group of cells or tissue that is formed by misregulated cellular proliferation. Tumors may show partial or complete lack of structural organization and functional coordination with the normal tissue, and usually form a distinct mass of tissue, which may be either benign or malignant.

By “metastasis” is meant the spread of cancer cells from its original site to another part of the body. The formation of metastasis is a very complex process and normally involves detachment of cancer cells from a primary tumor, entering the body circulation and settling down to grow within normal tissues elsewhere in the body. When tumor cells metastasize, the new tumor is called a secondary or metastatic tumor, and its cells normally resemble those in the original tumor. This means, for example, that, if breast cancer metastasizes to the lungs, the secondary tumor is made up of abnormal breast cells, not of abnormal lung cells. The tumor in the lung is then called metastatic breast cancer, not lung cancer.

The term “pharmaceutical composition” particularly refers to a composition suitable for administering to a human or animal, i.e., a composition containing components which are pharmaceutically acceptable. Preferably, a pharmaceutical composition comprises an active compound or a salt or prodrug thereof together with a carrier, diluent or pharmaceutical excipient such as buffer, preservative and tonicity modifier.

Numeric ranges described herein are inclusive of the numbers defining the range. The headings provided herein are not limitations of the various aspects or embodiments of this invention which can be read by reference to the specification as a whole. According to one embodiment, subject-matter described herein as comprising certain steps in the case of methods or as comprising certain ingredients in the case of compositions refers to subject-matter consisting of the respective steps or ingredients. It is preferred to select and combine preferred aspects and embodiments described herein and the specific subject-matter arising from a respective combination of preferred embodiments also belongs to the present disclosure.

### DETAILED DESCRIPTION OF THE INVENTION

The present invention is based on the development of fusion protein constructs in which IL-15 or variants thereof were fused to an anti-cancer antibody targeting MUC1. The established anti-MUC1 antibody exerts its anti-cancer activity by binding to tumor-associated MUC1 and recruiting and activating cytotoxic immune cells. The binding and activation of immune cells, in particular natural killer cells (NK cells) is achieved via the interaction of the antibody Fc part with Fcγ receptors, especially FcγRllla, on the immune cells. Upon activation, antibody-dependent cellular cytotoxicity (ADCC) is initiated. In view of this, the present inventors further improved efficacy of the established anti-MUC1 antibody by attaching IL-15 or a combination of IL-15 and the sushi domain of the IL-15 receptor α-chain to these antibodies. IL-15 is a cytokine which induces development, activation and proliferation of NK, NKT and T cells.

The inventors could demonstrate that a fusion construct of the anti-MUC1 antibody PankoMab and IL-15 effectively activates and recruits immune cells and induces lysis of tumor cells. Fine tuning of the activity can be achieved by using an active or inactive Fc part of the antibody, which either carries its natural glycosylation and is able to interact with Fc receptors, or is inactivated by deletion of the glycosylation site. Furthermore, also the activity of IL-15 can be controlled by using native IL-15 or a mutated version with decreased binding affinity to its receptor, as well as combining it with the sushi domain of the IL-15 receptor a subunit. Generally, the anti-MUC1 antibody with functional Fc part fused to native IL-15 without the sushi domain gave the best balance of target binding affinities, robust functionality and safety, with a high anti-tumor activity, a low off-target activity and favorable pharmacokinetic behavior with a long circulation half-life.

Furthermore, the inventors showed that IL-15 can be fused to the anti-MUC1 antibody at different locations, for example the heavy chain C terminus, the light chain C terminus and the light chain N terminus, which all gave functional fusion constructs. Surprisingly, the best activities as well as pharmacokinetic and pharmacodynamic parameters were obtained when fusing IL-15 to the C terminus of the antibody heavy chain.

In view of this, the present invention provides a fusion protein construct, comprising
(i) an antibody module specifically binding to MUC1, and
(ii) an IL-15 module.

### The Anti-MUC1 Antibody Module

The antibody module specifically binding to MUC1 (anti-MUC1 antibody module) comprises at least one antigen binding site specifically binding to an epitope of MUC1.

In certain embodiments, the antibody module comprises at least two, especially exactly two, antigen binding sites specifically binding to an epitope of MUC1. These antigen binding sites may be different or identical and in particular have the same amino acid sequence. In specific embodiments, the antigen binding sites of the antibody module comprise an antibody heavy chain variable region and an antibody light chain variable region.

In certain embodiments, the anti-MUC1 antibody module comprises at least one antibody heavy chain. In certain embodiments, the antibody module comprises two antibody heavy chains. The antibody heavy chains in particular comprise a VH domain, a CH1 domain, a hinge region, a CH2 domain and a CH3 domain. In certain other embodiments, the antibody heavy chains comprise a CH2 domain and a CH3 domain, but do not comprise a CH1 domain. In further embodiments, one or more constant domains of the heavy chains may be replaced by other domains, in particular similar domains such as for example albumin. The antibody heavy chains may be of any type, including γ-, α-, ε-, δ- and μ-chains, and preferably are γ-chains, including γ1, γ2, γ3- and γ4-chains, especially γ1-chains. Hence, the antibody module preferably is an IgG-type antibody module, in particular an IgG1-type antibody module.

In preferred embodiments, the antibody module comprises an Fc region. The antibody module may especially be a whole antibody, comprising two heavy chains each comprising the domains VH, CH1, hinge region, CH2 and CH3, and two light chains each comprising the domains VL and CL. The antibody module in particular is capable of binding to one or more human Fcγ receptors, especially human Fcγ receptor IIIA. In certain embodiments, the antibody module does not or not significantly bind to human Fcγ receptors. In these embodiments the antibody module in particular does not comprise a glycosylation site in the CH2 domain. In certain embodiments, the heavy chains of the antibody module do not comprise a C terminal lysine residue, e.g. the C terminal lysine encoded by the human gene for the γ1 antibody heavy chain. Furthermore, in some embodiments one or more of the last three amino acid residues at the C terminus of the heavy chain may be deleted and/or substituted. For example, the last two or the last three amino acids may be deleted, or the C terminal sequence PGK may be mutated, e.g. by substituting the lysine with alanine, the glycine with alanine or serine or the proline with leucine, or a combination thereof. The terms “heavy chain” and “CH3” as used herein include versions comprising such deletions and/or mutations.

In particular, the antibody module further comprises at least one antibody light chain, especially two antibody light chains. The antibody light chains in particular comprise a VL domain and a CL domain. The antibody light chain may be a κ-chain or a λ-chain and especially is a κ-chain. In certain embodiments, the antibody module comprises two antibody heavy chains and two antibody light chains.

In alternative embodiments, the antibody module does not comprise an antibody light chain. In these embodiments, the antibody heavy chains of the antibody module may additionally comprise a light chain variable region. In particular, the light chain variable region is fused to the N terminus of the heavy chain or is inserted C terminal to the heavy chain variable region. Peptide linkers may be present to connect the light chain variable region with the remaining parts of the heavy chain.

In specific embodiments, the antibody module comprises an antibody heavy chain variable region and an antibody light chain variable region. These variable regions may be covalently attached to each other, for example by a peptide linker. In certain embodiment, the antibody module comprises a polypeptide chain comprising—especially in the direction from N terminus to C terminus—an antibody heavy chain variable region, a peptide linker and an antibody light chain variable region. In particular, the antibody module may be a single chain variable fragment (scFv).

The anti-MUC1 antibody module specifically binds to an epitope of MUC1. The epitope may be specific for MUC1, i.e. it is not present on other molecules, or it may be an epitope also found on other molecules.

In certain embodiments, the antibody module binds to MUC1 in a glycosylation-dependent manner. In particular, the antibody module binds stronger to MUC1 if it is glycosylated, especially glycosylated in the extracellular tandem repeats. In specific embodiments, the antibody module binds stronger to MUC1 if it is O-glycosylated with N-acetyl galactosamine (Tn), sialyl α2-6 N-acetyl galactosamine (sTn), galactose β1-3 N-acetyl galactosamine (TF) or galactose βR1-3 (sialyl α2-6) N-acetyl galactosamine (sTF), preferably with Tn or TF.

In certain embodiments, the antibody module specifically binds to an epitope in the extracellular tandem repeats of MUC1. In particular, the antibody module binds stronger if said tandem repeats are glycosylated at a threonine residue with N-acetyl galactosamine (Tn), sialyl α2-6 N-acetyl galactosamine (sTn), galactose β1-3 N-acetyl galactosamine (TF) or galactose β1-3 (sialyl α2-6) N-acetyl galactosamine (sTF), preferably with Tn or TF. Preferably, the carbohydrate moiety is bound to the threonine residue by an α-O-glycosidic bond.

In particular embodiments, the antibody module is capable of specifically binding to an epitope in the tandem repeat domain of MUC1 which comprises the amino acid sequence PDTR (SEQ ID NO: 19) or PDTRP (SEQ ID NO: 20). The binding to this epitope preferably is glycosylation dependent, as described above, wherein in particular the binding is increased if the carbohydrate moiety described above is attached to the threonine residue of the sequence PDTR or PDTRP (SEQ ID NOs: 19 and 20), respectively.

In certain embodiments, the antibody module specifically binds a tumor-associated MUC1 epitope (TA-MUC1). A TA-MUC1 epitope in particular refers to an epitope of MUC1 which is present on tumor cells but not on normal cells and/or which is only accessible by antibodies in the host's circulation when present on tumor cells but not when present on normal cells. The epitopes described above, in particular those present in the tandem repeat domain of MUC1, may be tumor-associated MUC1 epitopes. In certain embodiments, the binding of the antibody module to cells expressing TA-MUC1 epitope is stronger than the binding to cells expressing normal, non-tumor MUC1. Preferably, said binding is at least 1.5-fold stronger, preferably at least 2-fold stronger, at least 5-fold stronger, at least 10-fold stronger or at least 100-fold stronger. In particular, TA-MUC1 is glycosylated with at least one N-acetyl galactosamine (Tn) or galactose β1-3 N-acetyl galactosamine (TF) in its extracellular tandem repeat region. In certain embodiments, the antibody module specifically binds to this epitope in the extracellular tandem repeat region of TA-MUC1 comprising N-acetyl galactosamine (Tn) or galactose β1-3 N-acetyl galactosamine (TF). Especially, said epitope comprises at least one PDTR or PDTRP (SEQ ID NO: 19 or 20) sequence of the MUC1 tandem repeats and is glycosylated at the threonine of the PDTR or PDTRP (SEQ ID NO: 19 or 20) sequence with N-acetyl galactosamine (Tn) or galactose β1-3 N-acetyl galactosamine (TF), preferably via an α-O-glycosidic bond. For TA-MUC1 binding, the antibody module preferably specifically binds the glycosylated MUC1 tumor epitope such that the strength of the bond is increased at least by a factor 2, preferably a factor of 4 or a factor of 10, most preferably a factor of 20 in comparison with the bond to the non-glycosylated peptide of identical length and identical peptide sequence.

In the following, specific embodiments of antibody modules specifically binding to TA-MUC1 are described.

In certain embodiments, the antibody module comprises at least one heavy chain variable region comprising the complementarity determining regions CDR-H1 having the amino acid sequence of SEQ ID NO: 1, CDR-H2 having the amino acid sequence of SEQ ID NO: 3 and CDR-H3 having the amino acid sequence of SEQ ID NO: 5, or comprising the complementarity determining regions CDR-H1 having the amino acid sequence of SEQ ID NO: 2, CDR-H2 having the amino acid sequence of SEQ ID NO: 4 and CDR-H3 having the amino acid sequence of SEQ ID NO: 6. According to one embodiment, the heavy chain variable region(s) present in the antibody module comprise(s) the amino acid sequence of SEQ ID NOs: 7, 8 or 9 or an amino acid sequence which is at least 75%, in particular at least 80%, at least 85%, at least 90%, at least 95% or at least 97% identical to one of said sequences. In certain embodiments, the heavy chain variable region of the antibody module comprises an amino acid sequence (i) which comprises a set of CDRs wherein CDR-H1 has the amino acid sequence of SEQ ID NO: 1, CDR-H2 has the amino acid sequence of SEQ ID NO: 3 and CDR-H3 has the amino acid sequence of SEQ ID NO: 5, or wherein CDR-H1 has the amino acid sequence of SEQ ID NO: 2, CDR-H2 has the amino acid sequence of SEQ ID NO: 4 and CDR-H3 has the amino acid sequence of SEQ ID NO: 6; and (ii) which is at least 80%, at least 85%, at least 90%, or at least 95% identical to any one of SEQ ID NOs: 7, 8 and 9.

The antibody module may further comprise at least one light chain variable region comprising the complementarity determining regions CDR-L1 having the amino acid sequence of SEQ ID NO: 10, CDR-L2 having the amino acid sequence of SEQ ID NO: 12 and CDR-L3 having the amino acid sequence of SEQ ID NO: 14, or comprising the complementarity determining regions CDR-L1 having the amino acid sequence of SEQ ID NO: 11, CDR-L2 having the amino acid sequence of SEQ ID NO: 13 and CDR-L3 having the amino acid sequence of SEQ ID NO: 15. According to one embodiment, the light chain variable region(s) present in the antibody module comprise(s) the amino acid sequence of SEQ ID NOs: 16, 17 or 18 or an amino acid sequence which is at least 75%, in particular at least 80%, at least 85%, at least 90%, at least 95% or at least 97% identical to one of said sequences. In certain embodiments, the light chain variable region of the antibody module comprises an amino acid sequence (i) which comprises a set of CDRs wherein CDR-L1 has the amino acid sequence of SEQ ID NO: 10, CDR-L2 has the amino acid sequence of SEQ ID NO: 12 and CDR-L3 has the amino acid sequence of SEQ ID NO: 14, or wherein CDR-L1 has the amino acid sequence of SEQ ID NO: 11, CDR-L2 has the amino acid sequence of SEQ ID NO: 13 and CDR-L3 has the amino acid sequence of SEQ ID NO: 15; and (ii) which is at least 80%, at least 85%, at least 90%, or at least 95% identical to any one of SEQ ID NOs: 16, 17 and 18.

In particular preferred embodiments, the antibody module comprises at least one, in particular two, heavy chain variable region comprising the amino acid sequence of SEQ ID NO: 9 and at least one, in particular two, light chain variable region comprising the amino acid sequence of SEQ ID NO: 18. In a further embodiment, the antibody module is derived from an antibody comprising one or more of the sequences described above, in particular from the antibody PankoMab in its chimeric or humanized version as described, for example, in WO 2004/065423 and WO 2011/012309, or from the antibody Gatipotuzumab.

The antibody module, wherein the CDR-H2 has the amino acid sequence of SEQ ID NO: 3 and/or wherein the heavy chain variable region has the amino acid sequence of SEQ ID NO: 8 or 9, has an N-glycosylation site in the heavy chain variable region. In certain embodiments, the antibody molecule comprises a mutation which removes this N-glycosylation site in the heavy chain variable region. In particular, the amino acid residue at position 8 of SEQ ID NO: 3 and/or at position 57 of SEQ ID NO: 9, respectively, is substituted by any other amino acid residue except Asn, especially by Gln or Ala. Therefore, in certain embodiments, the heavy chain variable region(s) present in the antibody module comprise(s) the complementarity determining regions CDR-H1 having the amino acid sequence of SEQ ID NO: 1, CDR-H2 having the amino acid sequence of SEQ ID NO: 33 and CDR-H3 having the amino acid sequence of SEQ ID NO: 5. In particular, the heavy chain variable region(s) present in the antibody module comprise(s) the amino acid sequence of SEQ ID NO: 34 or an amino acid sequence which is at least 75%, in particular at least 80%, at least 85%, at least 90%, at least 95% or at least 97% identical to one of said sequences. In certain embodiments, the heavy chain variable region(s) of the antibody module comprise(s) an amino acid sequence (i) which comprises a set of CDRs wherein CDR-H1 has the amino acid sequence of SEQ ID NO: 1, CDR-H2 has the amino acid sequence of SEQ ID NO: 33 and CDR-H3 has the amino acid sequence of SEQ ID NO: 5; and (ii) which is at least 80%, at least 85%, at least 90%, or at least 95% identical to any one of SEQ ID NO: 34. In these embodiments, the amino acid residue at position 8 of SEQ ID NO: 33 and position 57 of SEQ ID NO: 34 in particular is any amino acid residue except asparagine, especially glutamine or alanine. Furthermore, in these embodiments the light chain variable region(s) present in the antibody module in particular comprise(s) the complementarity determining regions CDR-L1 having the amino acid sequence of SEQ ID NO: 10, CDR-L2 having the amino acid sequence of SEQ ID NO: 12 and CDR-L3 having the amino acid sequence of SEQ ID NO: 14. In particular, the light chain variable region(s) present in the antibody module comprise(s) the amino acid sequence of SEQ ID NO: 18 or an amino acid sequence which is at least 75%, in particular at least 80%, at least 85%, at least 90%, at least 95% or at least 97% identical to one of said sequences. In these embodiments, the light chain variable region(s) of the antibody module especially comprise(s) an amino acid sequence (i) which comprises a set of CDRs wherein CDR-L1 has the amino acid sequence of SEQ ID NO: 10, CDR-L2 has the amino acid sequence of SEQ ID NO: 12 and CDR-L3 has the amino acid sequence of SEQ ID NO: 14; and (ii) which is at least 80%, at least 85%, at least 90%, or at least 95% identical to any one of SEQ ID NO: 18.

### The IL-15 Module

The IL-15 module of the fusion protein construct comprises one or more of the activities of IL-15. In particular, the IL-15 module is capable of specifically binding to the IL-2 receptor β- common γ-chain complex and/or to the IL-15 receptor α chain. In certain embodiments, the IL-15 module comprises IL-15 or a fragment thereof, especially human IL-15 or a fragment thereof. In specific embodiments, the IL-15 module comprises and in particular consists of human IL-15.

In specific embodiments, the IL-15 module comprises the sequence of SEQ ID NO: 21 or a sequence which is derived therefrom. In particular, the IL-15 module comprises an amino acid sequence which is at least 75%, in particular at least 80%, at least 85%, at least 90%, at least 95% or at least 97% identical to the sequence of SEQ ID NO: 21. In certain embodiments, the IL-15 module comprises and in particular consists of the amino acid sequence of SEQ ID NO: 21. In further embodiments, the IL-15 module comprises a fragment of said sequences, especially a fragment of at least 80, at least 90 or at least 100 amino acids in length. The fragment in particular retains the ability to specifically bind to the IL-2 receptor β- common γ-chain complex and/or to the IL-15 receptor α chain.

The IL-15 module may comprise a mutation which increases receptor binding. For example, the IL-15 module may comprise a substitution of asparagine to aspartic acid at an amino acid position corresponding to Asn72 of SEQ ID NO: 21. In embodiments wherein the IL-15 module comprises the sequence of SEQ ID NO: 21 or a sequence which is derived therefrom, the mutation which increases receptor binding is N72D. In alternative embodiments, the IL-15 module may comprise a mutation which decreases receptor binding. For example, the IL-15 module may comprise a substitution of isoleucine to glutamic acid at an amino acid position corresponding to lle67 of SEQ ID NO: 21. In embodiments wherein the IL-15 module comprises the sequence of SEQ ID NO: 21 or a sequence which is derived therefrom, the mutation which decreases receptor binding is I67E. The IL-15 module may be glycosylated at an amino acid corresponding to Asn79 and/or an amino acid corresponding to Asn112 of SEQ ID NO: 21.

In specific embodiments, the IL-15 module further comprises the IL-15 receptor α chain or a fragment thereof. The IL-15 receptor α chain in particular is human IL-15 receptor a chain. In certain embodiments, the IL-15 receptor α chain or the fragment thereof specifically binds to IL-15, especially to human IL-15. In specific embodiments, the IL-15 module comprises a fragment of the IL-15 receptor α chain which comprises or consists of only the extracellular domain or a part thereof of the IL-15 receptor α chain, especially of the human IL-15 receptor α chain. Especially, the fragment of the IL-15 receptor α chain comprises or consists of only the sushi domain of the IL-15 receptor a chain, especially of the human IL-15 receptor α chain.

In specific embodiments, the IL-15 module comprises a fragment of the IL-15 receptor α chain which comprises the sequence of SEQ ID NO: 22 or a sequence which is derived therefrom. In particular, the fragment of the IL-15 receptor α chain comprises an amino acid sequence which is at least 75%, in particular at least 80%, at least 85%, at least 90%, at least 95% or at least 97% identical to the sequence of SEQ ID NO: 22. In further embodiments, the fragment of the IL-15 receptor α chain comprises a fragment of said sequences, especially a fragment of at least 50, at least 55 or at least 60 amino acids in length. The fragment in particular retains the ability to specifically bind to the IL-2 receptor α- common γ-chain complex and/or to IL-15.

The IL-15 receptor α chain or fragment thereof may be part of the same polypeptide chain as the IL-15 or fragment thereof, or the IL-15 receptor α chain or fragment thereof and the IL-15 or fragment thereof may be part of different polypeptide chains. In preferred embodiments, the IL-15 receptor α chain or fragment thereof and the IL-15 or fragment thereof are part of the same polypeptide chain. In these embodiments, the IL-15 receptor α chain or fragment thereof may be fused to the N terminus or C terminus of the IL-15 or fragment thereof, especially to the N terminus thereof. In certain embodiments, the IL-15 receptor α chain or fragment thereof is fused to the IL-15 or fragment thereof via a peptide linker, in particular a peptide linker as described herein.

In specific embodiments, the IL-15 module as well as the entire fusion protein construct do not comprise the IL-15 receptor α chain or a fragment thereof which is capable of binding to IL-15.

In certain specific embodiments, the IL-15 module comprises and especially consists of human IL-15 having the amino acid sequence of SEQ ID NO: 21. In an alternative embodiment, the IL-15 module comprises and especially consists of human IL-15 having the amino acid sequence of SEQ ID NO: 21, wherein the isoleucine residue at position 67 is substituted with glutamic acid. In another embodiment, the IL-15 module comprises and especially consists of the human IL-15 receptor α chain fragment having the amino acid sequence of SEQ ID NO: 22 fused to the N terminus of human IL-15 having the amino acid sequence of SEQ ID NO: 21 via a peptide linker. In another embodiment, the IL-15 module has any of these designs, except that the human IL-15 has an amino acid sequence which is at least 90%, in particular at least 95% identical to SEQ ID NO: 21 over the entire length of the reference sequence, and/or the human IL-15 receptor α chain fragment has an amino acid sequence which is at least 90%, in particular at least 95% identical to SEQ ID NO: 22 over the entire length of the reference sequence, and wherein the IL-15 module specifically binds to the IL-2 receptor β- common γ-chain complex.

### The Fusion Protein Construct

The fusion protein construct comprises at least one anti-MUC1 antibody module and at least one IL-15 module. In certain embodiments, the fusion protein construct comprises at least two, in particular exactly two IL-15 modules. The IL-15 modules may be identical or different and in particular have the same amino acid sequence. In certain embodiments, at least one IL-15 module is fused to the C terminus of a heavy chain of the antibody module. Furthermore or alternative to this, at least one IL-15 module is fused to the C terminus of a light chain of the antibody module.

In specific embodiments wherein the fusion protein construct comprises two IL-15 modules, the antibody module also comprises two heavy chains and each of the IL-15 modules is fused to the C terminus of a different heavy chain of the antibody module. In alternative embodiments, wherein the fusion protein construct comprises two IL-15 modules, the antibody module also comprises two light chains and each of the IL-15 modules is fused to the C terminus of a different light chain of the antibody module. In further alternative embodiments, wherein the fusion protein construct comprises two IL-15 modules, the antibody module also comprises two light chains and each of the IL-15 modules is fused to the N terminus of a different light chain of the antibody module.

The IL-15 module may be fused to the antibody module directly via a peptide bond or indirectly via a peptide linker. A direct fusion refers to embodiments wherein the sequence of the IL-15 module directly follows the sequence of the antibody module without any intermediate amino acids between these two sequences. A fusion via a peptide linker refers to embodiments wherein one or more amino acids are present between the sequence of the antibody module and the sequence of the IL-15 module. These one or more amino acids form the peptide linker between the antibody module and the IL-15 module.

The peptide linker may in principle have any number of amino acids and any amino acid sequence which are suitable for linking the antibody module and the IL-15 module. In certain embodiments, the peptide linker comprises at least 3, preferably at least 5, at least 8, at least 10, at least 15 or at least 20 amino acids. In further embodiments, the peptide linker comprises 50 or less, preferably 45 or less, 40 or less, 35 or less, 30 or less, 25 or less or 20 or less amino acids. In particular, the peptide linker comprises from 10 to 30 amino acids, especially 20 or 30 amino acids. In specific embodiments, the peptide linker consists of glycine and serine residues. Glycine and serine may be present in the peptide linker in a ratio of 2 to 1, 3 to 1, 4 to 1 or 5 to 1 (number of glycine residues to number of serine residues). For example, the peptide linker may comprise a sequence of four glycine residues followed by one serine residue, and in particular 1, 2, 3, 4, 5 or 6 repeats of this sequence. Specific examples are peptide linkers comprising or consisting of the amino acid sequence GGGGS (SEQ ID NO: 31), 2 repeats of the amino acid sequence GGGGS (SEQ ID NO: 31), 3 repeats of the amino acid sequence GGGGS (SEQ ID NO: 31), 4 repeats of the amino acid sequence GGGGS (SEQ ID NO: 31) and 6 repeats of the amino acid sequence GGGGS (SEQ ID NO: 31). Especially peptide linkers consisting of 2, 3 or 4 repeats of the amino acid sequence GGGGS (SEQ ID NO: 31) may be used. In specific embodiments, the fusion protein construct comprises a peptide linker comprising 2, 3 or 4 repeats of the amino acid sequence GGGGS (SEQ ID NO: 31) between a C terminus of the antibody module and the N terminus of the IL-15 module and/or a peptide linker comprising 4 or 6 repeats of the amino acid sequence GGGGS (SEQ ID NO: 31) between the IL-15 or fragment thereof and the IL-15 receptor α chain or fragment thereof of the IL-15 module. In further embodiments, the peptide linker comprises the sequence PAPAP (SEQ ID NO: 32), and in particular 3 or 6 repeats of this sequence. In specific embodiments, the fusion protein construct comprises a peptide linker comprising 3 or 6 repeats of the amino acid sequence PAPAP (SEQ ID NO: 32) between a C terminus of the antibody module and the N terminus of the IL-15 module.

In other embodiments the peptide linker comprises sequences which show no or only minor immunogenic potential in humans, preferably sequences which are human sequences or naturally occurring sequences. In a further preferred embodiment the peptide linker and the adjacent amino acids show no or only minor immunogenic potential. Peptide linkers as described above may also be used to link other elements of the fusion protein construct, such as a heavy chain variable region and a light chain variable region present in one antigen binding fragment.

In certain embodiments, the IL-15 module is fused to the C terminus of a heavy chain of the antibody module via a peptide linker. In these embodiments, the peptide linker may comprise an additional amino acid residue at its N terminus, in particular a proline residue, an aspartate residue or an alanine residue. Additionally or alternatively, the last 1, 2 or 3 amino acid residues of the antibody heavy chain may be deleted and/or mutated. Specific examples include fusion protein constructs wherein the peptide linker comprises an additional proline residue or aspartate residue at its N terminus; fusion protein constructs wherein the peptide linker comprises an additional alanine residue at its N terminus and the last amino acid residue of the antibody heavy chain is deleted; and fusion protein constructs wherein the last two amino acid residues of the antibody heavy chain are deleted. In these embodiments, the peptide linker especially comprises 2, 3 or 4 repeats of the amino acid sequence GGGGS (SEQ ID NO: 31) or 3 or 6 repeats of the amino acid sequence PAPAP (SEQ ID NO: 32).

The fusion protein construct in particular is an antibody construct. The antibody construct specifically binds to an epitope of MUC1, but does not comprise any further antigen binding sites specifically binding to another antigen. In alternative embodiments, the fusion protein construct comprises one or more additional antigen binding sites specifically binding other antigens. These additional antigen binding sites may be present anywhere in the fusion protein construct. In certain embodiments, an additional antigen binding site is present in an antigen binding fragment fused to the C or N terminus of an antibody light chain or heavy chain of the antibody module. In particular, if the antibody module comprises two antibody light chains, one or more antigen binding fragments, especially one additional antigen binding fragment, may be fused to the C or N terminus, especially C terminus, of each of the antibody light chains of the antibody module. These additional antigen binding fragments may be identical or different, and in particular have the same amino acid sequence. In these embodiments, the IL-15 module is preferably fused to the C terminus of the antibody heavy chain(s) of the antibody module. Furthermore, if the antibody module comprises two antibody heavy chains, one or more additional antigen binding fragments, especially one additional antigen binding fragment, may be fused to the C terminus of each of the antibody heavy chains of the antibody module. These additional antigen binding fragments may be identical or different, and in particular have the same amino acid sequence. In these embodiments, the IL-15 module is preferably fused to the C terminus of the antibody light chain(s) of the antibody module.

In specific embodiments, the additional antigen binding fragment comprises an antibody heavy chain variable region and an antibody light chain variable region. These variable regions may be covalently attached to each other, for example by a peptide linker. In certain embodiment, the additional antigen binding fragment comprises a polypeptide chain comprising—especially in the direction from N terminus to C terminus—an antibody heavy chain variable region, a peptide linker and an antibody light chain variable region. In particular, the additional antigen binding fragment may be a single chain variable fragment (scFv).

The additional antigen binding site may specifically bind to any antigen, especially to tumor-associated antigens or checkpoint antigens of immune cells. Suitable examples of such antigens may be selected from the group consisting of CD3, EGFR, HER2, PD-1, PD-L1, CD40, CEA, EpCAM, CD7, CD28, GITR, ICOS, OX40, 4-1BB, CTLA-4, TFa, LeY, CD160, Galectin-3, and Galectin-1.

In specific embodiments, the additional antigen binding fragment specifically binds to CD3. In particular, the additional antigen binding fragment is a single chain variable region fragment (scFv) specifically binding to CD3. The additional antigen binding fragment specifically binds to an epitope of CD3. In particular, the additional antigen binding fragment specifically binds to CD3ε. In specific embodiments, the additional antigen binding fragment specifically binds to CD3ε n a conformation-dependent manner, especially only if it is in complex with CD3δ.

In certain embodiments, the additional antigen binding fragment specifically binding to CD3 comprises at least one heavy chain variable region comprising the complementarity determining regions CDR-H1 having the amino acid sequence of SEQ ID NO: 23, CDR-H2 having the amino acid sequence of SEQ ID NO: 24 and CDR-H3 having the amino acid sequence of SEQ ID NO: 25. According to one embodiment, the heavy chain variable region(s) present in the additional antigen binding fragment comprise(s) the amino acid sequence of SEQ ID NOs: 26 or an amino acid sequence which is at least 75%, in particular at least 80%, at least 85%, at least 90%, at least 95% or at least 97% identical to one of said sequences. In certain embodiments, the heavy chain variable region of the additional antigen binding fragment comprises an amino acid sequence (i) which comprises a set of CDRs wherein CDR-H1 has the amino acid sequence of SEQ ID NO: 23, CDR-H2 has the amino acid sequence of SEQ ID NO: 24 and CDR-H3 has the amino acid sequence of SEQ ID NO: 25; and (ii) which is at least 80%, at least 85%, at least 90%, or at least 95% identical to any one of SEQ ID NOs: 26.

The additional antigen binding fragment specifically binding to CD3 may further comprise at least one light chain variable region comprising the complementarity determining regions CDR-L1 having the amino acid sequence of SEQ ID NO: 27, CDR-L2 having the amino acid sequence of SEQ ID NO: 28 and CDR-L3 having the amino acid sequence of SEQ ID NO: 29. According to one embodiment, the light chain variable region(s) present in the additional antigen binding fragment comprise(s) the amino acid sequence of SEQ ID NOs: 30 or an amino acid sequence which is at least 75%, in particular at least 80%, at least 85%, at least 90%, at least 95% or at least 97% identical to one of said sequences. In certain embodiments, the light chain variable region of the additional antigen binding fragment comprises an amino acid sequence (i) which comprises a set of CDRs wherein CDR-L1 has the amino acid sequence of SEQ ID NO: 27, CDR-L2 has the amino acid sequence of SEQ ID NO: 28 and CDR-L3 has the amino acid sequence of SEQ ID NO: 29 and (ii) which is at least 80%, at least 85%, at least 90%, or at least 95% identical to any one of SEQ ID NOs: 30.

In particular preferred embodiments, the additional antigen binding fragment specifically binding to CD3 comprises at least one, in particular one, heavy chain variable region comprising the amino acid sequence of SEQ ID NO: 26 and at least one, in particular one, light chain variable region comprising the amino acid sequence of SEQ ID NO: 30.

In certain embodiments, the fusion protein construct comprises one or more further agents conjugated thereto. The further agent may be any agent suitable for conjugation to the fusion protein construct. If more than one further agent is present in the fusion protein construct, these further agents may be identical or different, and in particular are all identical. Conjugation of the further agent to the fusion protein construct can be achieved using any methods known in the art. The further agent may be covalently, in particular by fusion or chemical coupling, or non-covalently attached to the fusion protein construct. In certain embodiments, the further agent is covalently attached to the fusion protein construct, especially via a linker moiety. The linker moiety may be any chemical entity suitable for attaching the further agent to the fusion protein construct.

In certain embodiments, the further agent is a polypeptide of protein. This polypeptide or protein may in particular be fused to a polypeptide chain of the antibody module or a polypeptide chain of the IL-15 module. In certain embodiments, the further agent being a polypeptide or protein is fused to the C or N terminus of an antibody light chain or antibody heavy chain of the antibody module. In embodiments wherein the antibody module comprises two antibody light chains, a further agent being a polypeptide or protein may be fused to the C or N terminus, especially the C terminus, of each of the two antibody light chains. In embodiments wherein the antibody module comprises two antibody heavy chains, a further agent being a polypeptide or protein may be fused to the C terminus of each of the two antibody heavy chains. The polypeptide or protein may be identical or different and in particular have the same amino acid sequence. Suitable examples of such further agents being a polypeptide or protein may be selected from the group consisting of cytokines, chemokines, antibody modules, antigen binding fragments, enzymes, and interaction domains.

The further agent preferably is useful in therapy, diagnosis, prognosis and/or monitoring of a disease, in particular cancer. For example, the further agent may be selected from the group consisting of radionuclides, chemotherapeutic agents, detectable labels, toxins, cytolytic components, immunomodulators, immunoeffectors, and liposomes.

### Glycosylation of the Fusion Protein Construct

The anti-MUC1 antibody module may comprise a CH2 domain in one or more antibody heavy chains. Natural human antibodies of the IgG type comprise an N-glycosylation site in the CH2 domain. The CH2 domains present in the antibody module may or may not comprise an N-glycosylation site.

In certain embodiments, the CH2 domains present in the antibody module do not comprise an N-glycosylation site. In particular, the antibody module does not comprise an asparagine residue at the position in the heavy chain corresponding to position 297 according to the IMGT/Eu numbering system. For example, the antibody module may comprise an Ala297 mutation in the heavy chain. In these embodiments, the fusion protein construct preferably has a strongly reduced ability or completely lacks the ability to induce, via binding to Fcγ receptors, antibody-dependent cellular cytotoxicity (ADCC) and/or antibody-dependent cellular phagocytosis (ADCP) and/or complement-dependent cytotoxicity (CDC). Strongly reduced ability in this respect in particular refers to a reduction to 10% or less, especially 3% or less, 1% or less or 0.1% or less activity compared to the same fusion protein construct comprising an N-glycosylation site in its CH2 domains and having a common mammalian glycosylation pattern such as those obtainable by production in human cell lines or in CHO or SP2/0 cell lines, for example a glycosylation pattern as described herein.

Via the presence or absence of N-glycosylation at the CH2 domain and the glycosylation pattern, the activation of T cells and NK cells by and the cytotoxicity of the fusion protein construct can be controlled. Even without glycosylation at the CH2 domain, immune cells are activated at the tumor site by the IL-15 module of the fusion protein construct. With CH2 glycosylation, immune cell activation is increased, and with a glycosylation pattern with reduced fucosylation, immune cell activation is even more pronounced.

In alternative embodiments, the CH2 domains present in the antibody module comprise an N-glycosylation site. This glycosylation site in particular is at an amino acid position corresponding to amino acid position 297 of the heavy chain according to the IMGT/Eu numbering system and has the amino acid sequence motive Asn Xaa Ser/Thr wherein Xaa may be any amino acid except proline. The N-linked glycosylation at Asn297 is conserved in mammalian IgGs as well as in homologous regions of other antibody isotypes. Due to optional additional amino acids which may be present in the variable region or other sequence modifications, the actual position of this conserved glycosylation site may vary in the amino acid sequence of the antibody. Preferably, the glycans attached to the antibody module are biantennary complex type N-linked carbohydrate structures, preferably comprising at least the following structure:

Asn-GlcNAc-GlcNAc-Man-(Man-GlcNAc)₂

wherein Asn is the asparagine residue of the polypeptide portion of the antibody module; GlcNAc is N-acetylglucosamine and Man is mannose. The terminal GlcNAc residues may further carry a galactose residue, which optionally may carry a sialic acid residue. A further GlcNAc residue (named bisecting GlcNAc) may be attached to the Man nearest to the polypeptide. A fucose may be bound to the GlcNAc attached to the Asn.

The fusion protein construct may have a glycosylation pattern at the CH2 domains of the antibody module having a high amount of core fucose or a low amount of core fucose. A reduced amount of fucosylation at the CH2 domains increases the ability of the fusion protein construct to induce ADCC. In certain embodiments, the relative amount of glycans carrying a core fucose residue is 40% or less, especially 30% or less or 20% or less of the total amount of glycans attached to the CH2 domains of the antibody module in a composition. In alternative embodiments, the relative amount of glycans carrying a core fucose residue is at least 60%, especially at least 65% or at least 70% of the total amount of glycans attached to the CH2 domains of the antibody module in a composition.

Via the presence or absence of the glycosylation site in the CH2 domain of the anti-MUC1 antibody module and the presence or absence of fucose in the glycan structures at said glycosylation site, the ability of the fusion protein construct to induce ADCC via the Fc part of the antibody module and the strength of said ADCC induction can be controlled. Cytotoxicity mediated by T cells and NK cells is already initiated by proliferation and activation of said immune cells at the tumor site. This is achieved by the anti-MUC1 antibody module which binds to the tumor cells and locates the fusion protein construct to the tumor site, and the IL-15 module which induces proliferation and activation of T cells and NK cells. The overall cytotoxic activity as mediated by T cells and NK cells may be increased by glycosylation of the Fc part of the antibody module and further by reducing the amount of fucosylation in said glycosylation. With Fc-glycosylation, in particular with low fucosylation, ADCC mediated by NK cells is further enhanced. In certain applications, fine tuning of the ADCC activity is important. Therefore, in certain situations, the fusion protein construct without a glycosylation site in the CH2 domain of the antibody module, the fusion protein construct with a glycosylation site in the CH2 domain of the antibody module and with a high amount of fucosylation, or the fusion protein construct with a glycosylation site in the CH2 domain of the antibody module and with a low amount of fucosylation may be most advantageous.

In certain embodiments, the IL-15 module is glycosylated. In particular, the IL-15 module may be glycosylated at an amino acid corresponding to Asn79 and/or Asn112 of SEQ ID NO: 21.

The fusion protein construct is preferably recombinantly produced in a host cell. The host cell used for the production of the fusion protein construct may be any host cells which can be used for antibody production. Suitable host cells are in particular eukaryotic host cells, especially mammalian host cells. Exemplary host cells include yeast cells such as Pichia pastoris cell lines, insect cells such as SF9 and SF21 cell lines, plant cells, bird cells such as EB66 duck cell lines, rodent cells such as CHO, NS0, SP2/0 and YB2/0 cell lines, and human cells such as HEK293, PER.C6, CAP, CAP-T, AGE1.HN, Mutz-3 and KG1 cell lines.

In certain embodiments, the fusion protein construct is produced recombinantly in a human blood cell line, in particular in a human myeloid leukemia cell line. Preferred human cell lines which can be used for production of the fusion protein construct as well as suitable production procedures are described in WO 2008/028686 A2. In a specific embodiment, the fusion protein construct is obtained by expression in a human myeloid leukemia cell line selected from the group consisting of NM-H9D8, NM-H9D8-E6 and NM-H9D8-E6Q12. These cell lines were deposited under the accession numbers DSM ACC2806 (NM-H9D8; deposited on Sep. 15, 2006), DSM ACC2807 (NM-H9D8-E6; deposited on Oct. 5, 2006) and DSM ACC2856 (NM-H9D8-E6Q12; deposited on Aug. 8, 2007) according to the requirements of the Budapest Treaty at the Deutsche Sammlung von Mikroorganismen and Zellkulturen (DSMZ), Inhoffenstraße 7B, 38124 Braunschweig (DE) by Glycotope GmbH, Robert-Rössle-Str. 10, 13125 Berlin (DE). NM-H9D8 cells provide a glycosylation pattern with a high degree of sialylation, a high degree of bisecting GlycNAc, a high degree of galactosylation and a high degree of fucosylation. NM-H9D8-E6 and NM-H9D8-E6Q12 cells provide a glycosylation pattern similar to that of NM-H9D8 cells, except that the degree of fucosylation is very low. Other suitable cell lines include K562, a human myeloid leukemia cell line present in the American Type Culture Collection (ATCC CCL-243), as well as cell lines derived from the aforementioned.

In further embodiments, the fusion protein construct is produced recombinantly in a CHO cell line, especially a CHO dhfr− cell line such as the cell line of ATCC No. CRL-9096.

### The Nucleic Acid, Expression Cassette, Vector, Cell Line and Composition

In a further aspect, the present invention provides a nucleic acid encoding the fusion protein construct. The nucleic acid sequence of said nucleic acid may have any nucleotide sequence suitable for encoding the fusion protein construct. However, preferably the nucleic acid sequence is at least partially adapted to the specific codon usage of the host cell or organism in which the nucleic acid is to be expressed, in particular the human codon usage. The nucleic acid may be double-stranded or single-stranded DNA or RNA, preferably double-stranded DNA such as cDNA or single-stranded RNA such as mRNA. It may be one consecutive nucleic acid molecule or it may be composed of several nucleic acid molecules, each coding for a different part of the fusion protein construct.

If the fusion protein construct is composed of more than one different amino acid chain, such as a light chain and a heavy chain of the antibody module, the nucleic acid may, for example, be a single nucleic acid molecule containing several coding regions each coding for one of the amino acid chains of the fusion protein construct, preferably separated by regulatory elements such as IRES elements in order to generate separate amino acid chains, or the nucleic acid may be composed of several nucleic acid molecules wherein each nucleic acid molecule comprises one or more coding regions each coding for one of the amino acid chains of the fusion protein construct. In addition to the coding regions encoding the fusion protein construct, the nucleic acid may also comprise further nucleic acid sequences or other modifications which, for example, may code for other proteins, may influence the transcription and/or translation of the coding region(s), may influence the stability or other physical or chemical properties of the nucleic acid, or may have no function at all.

In a further aspect, the present invention provides an expression cassette or vector comprising a nucleic acid according to the invention and a promoter operatively connected with said nucleic acid. In addition, the expression cassette or vector may comprise further elements, in particular elements which are capable of influencing and/or regulating the transcription and/or translation of the nucleic acid, the amplification and/or reproduction of the expression cassette or vector, the integration of the expression cassette or vector into the genome of a host cell, and/or the copy number of the expression cassette or vector in a host cell. Suitable expression cassettes and vectors comprising respective expression cassettes for expressing antibodies are well known in the prior art and thus, need no further description here.

Furthermore, the present invention provides a host cell comprising the nucleic acid according to the invention or the expression cassette or vector according to the invention. The host cell may be any host cell. It may be an isolated cell or a cell comprised in a tissue. Preferably, the host cell is a cultured cell, in particular a primary cell or a cell of an established cell line, preferably a tumor-derived cell. Preferably, it is a bacterial cell such as E. coli, a yeast cell such as a Saccharomyces cell, in particular S. cerevisiae, an insect cell such as a Sf9 cell, or a mammalian cell, in particular a human cell such as a tumor-derived human cell, a hamster cell such as CHO, or a primate cell. In a preferred embodiment of the invention the host cell is derived from human myeloid leukaemia cells. Preferably, it is selected from the following cells or cell lines: K562, KG1, MUTZ-3 or a cell or cell line derived therefrom, or a mixture of cells or cell lines comprising at least one of those aforementioned cells. The host cell is preferably selected from the group consisting of NM-H9D8, NM-H9D8-E6, NM H9D8-E6Q12, and a cell or cell line derived from anyone of said host cells, or a mixture of cells or cell lines comprising at least one of those aforementioned cells. These cell lines and their properties are described in detail in the PCT-application WO 2008/028686 A2. In preferred embodiments, the host cell is optimized for expression of glycoproteins, in particular antibodies, having a specific glycosylation pattern. Preferably, the codon usage in the coding region of the nucleic acid according to the invention and/or the promoter and the further elements of the expression cassette or vector are compatible with and, more preferably, optimized for the type of host cell used. Preferably, the fusion protein construct is produced by a host cell or cell line as described above.

In another aspect, the present invention provides a composition comprising the fusion protein construct, the nucleic acid, the expression cassette or vector, or the host cell. The composition may also contain more than one of these components. Furthermore, the composition may comprise one or more further components selected from the group consisting of solvents, diluents, and excipients. Preferably, the composition is a pharmaceutical composition. In this embodiment, the components of the composition preferably are all pharmaceutically acceptable. The composition may be a solid or fluid composition, in particular a—preferably aqueous—solution, emulsion or suspension or a lyophilized powder.

### Use in Medicine

The fusion protein construct in particular is useful in medicine, in particular in therapy, diagnosis, prognosis and/or monitoring of a disease, in particular a disease as described herein, preferably cancer, infections and immunodeficiencies.

Therefore, in a further aspect, the invention provides the fusion protein construct, the nucleic acid, the expression cassette or vector, the host cell, or the composition for use in medicine. Preferably, the use in medicine is a use in the treatment, prognosis, diagnosis and/or monitoring of a disease such as, for example, diseases associated with abnormal cell growth such as cancer, infections such as bacterial, viral, fungal or parasitic infections, and diseases associated with a reduce immune activity such as immunodeficiencies. In a preferred embodiment, the disease is cancer. Preferably the cancer is selected from the group consisting of ovarian cancer, breast cancer such as triple negative breast cancer, lung cancer and pancreatic cancer. The cancer may further in particular be selected from colon cancer, stomach cancer, liver cancer, kidney cancer, bladder cancer, skin cancer, cervix cancer, prostate cancer, 2 5 gastrointestinal cancer, endometrial cancer, thyroid cancer and blood cancer.

In certain embodiments, the viral infection is caused by human immunodeficiency virus, herpes simplex virus, Epstein Barr virus, influenza virus, lymphocytic choriomeningitis virus, hepatitis B virus or hepatitis C virus. In certain embodiments, the disease comprises or is associated with cells which express MUC1. For example, a cancer to be treated is MUC1 positive, i.e. comprises cancer cells which express MUC1.

In specific embodiments, the fusion protein construct is used in combination with another therapeutic agent, especially another anti-cancer agent. Said further therapeutic agent may be any known anti-cancer drug. Suitable anti-cancer therapeutic agents which may be combined with the fusion protein construct may be chemotherapeutic agents, antibodies, immunostimulatory agents, cytokines, chemokines, and vaccines. Furthermore, therapy with the fusion protein construct may be combined with radiation therapy, surgery and/or traditional Chinese medicine.

In certain embodiments, the fusion protein construct is for use in the treatment of cancer in combination with one or more of the following
(i) a cellular therapy, e.g., CAR-T, TCR, NK, or CD-based cell therapy;
(ii) an immune activating antibody, e.g. bispecific T or NK cell engager or other immunocytokines;
(iii) a checkpoint antibody, e.g., antagonistic or agonistic checkpoint antibodies, such as antibodies against CD3, PD-1, PD-L1, CD40, CD7, CD28, GITR, ICOS, OX40, 4-1BB, CTLA-4, CD160, Galectin-3, and Galectin-1;
(iv) vaccination therapy;
(v) chemotherapy;
(vi) a tumor-targeting antibody, including but not limited to ADCC-mediating monoclonal antibodies, such as antibodies against EGFR, HER2, TFα, LeY, CEA and EpCAM;
(vii) a therapy which up-regulates TA-MUC1 on the surface of the cancer cells, e.g., via inhibition of EGFR.

In specific embodiments, the fusion protein construct is for use in the treatment of cancer in combination with a bispecific antibody targeting MUC1 and CD3, especially a bispecific antibody comprising an antibody module specifically binding to MUC1 and an antigen binding fragment specifically binding to CD3. The antibody module specifically binding to MUC1 of the bispecific antibody in particular is as described herein for the usion protein construct and the antigen binding fragment specifically binding to CD3 of the bispecific antibody in particular is as described herein for the additional antigen binding fragment specifically binding to CD3. Suitable bispecific antibodies are described, for example, in WO 2018/178047 (PCT/EP2018/057721).

In further embodiments, the fusion protein construct is for use in the treatment of cancer in combination with an antibody against PD-L1. In particular, a combination of the fusion protein construct and an antibody against PD-L1 shows synergistic effects in tumor cell killing and/or immune cell activation, especially T cell activation. Exemplary antibodies against PD-L1 are described, for example, in WO 2018/178122 (PCT/EP2018/057844).

In further embodiments, the fusion protein construct is for use in the treatment of cancer in combination with an antibody against EGFR. In particular, a combination of the fusion protein construct and an antibody against EGFR shows synergistic effects in tumor cell killing. Exemplary antibodies against EGFR are tomuzotuximab and cetuximab.

In further embodiments, the fusion protein construct is for use in the treatment of cancer in combination with an antibody against CD40. Treatment with an anti-CD40 antibody up-regulates expression of the IL-15 receptor subunits on immune cells of the patient. Thereby, immune cell activation and tumor treatment with the fusion protein construct are enhanced in patients treated with an anti-CD40 antibody. Exemplary antibodies against CD40 are described, for example, in WO 2018/178046 (PCT/EP2018/057717).

### Specific Embodiments

In the following, specific embodiments of the present invention are described.

Embodiment 1. A fusion protein construct, comprising
(i) an antibody module specifically binding to MUC1 (anti-MUC1 antibody module), and
(ii) an IL-15 module.

Embodiment 2. The fusion protein construct according to Embodiment 1, wherein the anti-MUC1 antibody module comprises two heavy chains, each comprising a VH domain, a CH1 domain, a hinge region, a CH2 domain and a CH3 domain.

Embodiment 3. The fusion protein construct according to Embodiment 1 or 2, wherein the anti-MUC1 antibody module comprises two light chains, each comprising a

VL domain and a CL domain.

Embodiment 4. The fusion protein construct according to any one of Embodiments 1 to 3, wherein the anti-MUC1 antibody module is an IgG-type antibody module, in particular an IgG1-type antibody module.

Embodiment 5. The fusion protein construct according to any one of Embodiments 1 to 4, wherein the anti-MUC1 antibody module has a κ-chain.

Embodiment 6. The fusion protein construct according to any one of Embodiments 1 to 5, wherein the anti-MUC1 antibody module specifically binds to a TA-MUC1 epitope.

Embodiment 7. The fusion protein construct according to any one of Embodiments 1 to 6, wherein the anti-MUC1 antibody module comprises a set of heavy chain CDR sequences with CDR-H1 having the amino acid sequence of SEQ ID NO: 1, CDR-H2 having the amino acid sequence of SEQ ID NO: 3 and CDR-H3 having the amino acid sequence of SEQ ID NO: 5, or CDR-H1 having the amino acid sequence of SEQ ID NO: 2, CDR-H2 having the amino acid sequence of SEQ ID NO: 4 and CDR-H3 having the amino acid sequence of SEQ ID NO: 6.

Embodiment 8. The fusion protein construct according to any one of Embodiments 1 to 7, wherein the anti-MUC1 antibody module comprises an antibody heavy chain variable region sequence which is at least 80% identical to any one of SEQ ID NOs: 7, 8 and 9.

Embodiment 9. The fusion protein construct according to any one of Embodiments 1 to 6, wherein the anti-MUC1 antibody module comprises a set of heavy chain CDR sequences with CDR-H1 having the amino acid sequence of SEQ ID NO: 1, CDR-H2 having the amino acid sequence of SEQ ID NO: 3 and CDR-H3 having the amino acid sequence of SEQ ID NO: 5.

Embodiment 10. The fusion protein construct according to any one of Embodiments 1 to 6 and 9, wherein the anti-MUC1 antibody module comprises an antibody heavy chain variable region sequence which is at least 80% identical to SEQ ID NO: 9.

Embodiment 11. The fusion protein construct according to any one of Embodiments 1 to 6, wherein the anti-MUC1 antibody module comprises a set of heavy chain CDR sequences with CDR-H1 having the amino acid sequence of SEQ ID NO: 1, CDR-H2 having the amino acid sequence of SEQ ID NO: 33 and CDR-H3 having the amino acid sequence of SEQ ID NO: 5.

Embodiment 12. The fusion protein construct according to any one of Embodiments 1 to 6 and 11, wherein the anti-MUC1 antibody module comprises an antibody heavy chain variable region sequence which is at least 80% identical to SEQ ID NO: 34.

Embodiment 13. The fusion protein construct according to any one of Embodiments 1 to 12, wherein the anti-MUC1 antibody module comprises a set of light chain CDR sequences with CDR-L1 having the amino acid sequence of SEQ ID NO: 10, CDR-L2 having the amino acid sequence of SEQ ID NO: 12 and CDR-L3 having the amino acid sequence of SEQ ID NO: 14.

Embodiment 14. The fusion protein construct according to any one of Embodiments 1 to 13, wherein the anti-MUC1 antibody module comprises an antibody light chain variable region sequence which is at least 80% identical to SEQ ID NO: 18.

Embodiment 15. The fusion protein construct according to any one of Embodiments 1 to 14, wherein the IL-15 module comprises IL-15 or a fragment thereof, especially human IL-15 or a fragment thereof.

Embodiment 16. The fusion protein construct according to Embodiment 15, wherein the IL-15 module comprises full-length human IL-15.

Embodiment 17. The fusion protein construct according to Embodiment 15 or 16, wherein human IL-15 has the amino acid sequence of SEQ ID NO: 21.

Embodiment 18. The fusion protein construct according to any one of Embodiments 1 to 17, wherein the IL-15 module comprises a mutation decreasing receptor binding.

Embodiment 19. The fusion protein construct according to Embodiment 18, wherein the mutation decreasing receptor binding is a substitution of isoleucine to glutamate at the position corresponding to 11e67 in SEQ ID NO: 21.

Embodiment 20. The fusion protein construct according to any one of Embodiments 1 to 19, wherein the IL-15 module specifically binds to an interleukin receptor comprising the IL-2 receptor β-chain, the common γ-chain and the IL-15 receptor α chain.

Embodiment 21. The fusion protein construct according to any one of Embodiments 1 to 20, wherein the IL-15 module specifically binds to an interleukin receptor comprising the human IL-2 receptor β-chain, the human common γ-chain and the human IL-15 receptor α chain.

Embodiment 22. The fusion protein construct according to any one of Embodiments 15 to 21, wherein the IL-15 module further comprises an IL-15 receptor α chain or a fragment thereof, especially human IL-15 receptor α chain or a fragment thereof.

Embodiment 23. The fusion protein construct according to Embodiment 22, wherein the fragment of the IL-15 receptor α chain is the extracellular domain of the human IL-15 receptor α chain or a part thereof.

Embodiment 24. The fusion protein construct according to Embodiment 22, wherein the fragment of the IL-15 receptor α chain is the sushi domain of the human IL-15 receptor α chain or a part thereof.

Embodiment 25. The fusion protein construct according to any one of Embodiments 22 to 24, wherein IL-15 receptor α chain or the fragment thereof comprises the sequence of SEQ ID NO: 22.

Embodiment 26. The fusion protein construct according to any one of Embodiments 22 to 25, wherein IL-15 receptor α chain or the fragment thereof specifically binds to human IL-15.

Embodiment 27. The fusion protein construct according to any one of Embodiments 22 to 26, wherein IL-15 receptor α chain or the fragment thereof is fused to the N terminus of human IL-15 or the fragment thereof.

Embodiment 28. The fusion protein construct according to any one of Embodiments 22 to 27, wherein IL-15 receptor α chain or the fragment thereof is fused to the human IL-15 or the fragment thereof via a peptide linker.

Embodiment 29. The fusion protein construct according to Embodiment 28, wherein the peptide linker comprises the amino acid sequence of SEQ ID NO: 31, in particular 2 or more, especially 3 or 4, repeats of the amino acid sequence of SEQ ID NO: 31.

Embodiment 30. The fusion protein construct according to Embodiment 29, wherein the peptide linker consists of 2, 3 or 4 repeats of the amino acid sequence of SEQ ID NO: 31.

Embodiment 31. The fusion protein construct according to any one of Embodiments 1 to 14, wherein the IL-15 module has the amino acid sequence of SEQ ID NO: 21.

Embodiment 32. The fusion protein construct according to any one of Embodiments 1 to 14, wherein the IL-15 module comprises the amino acid sequence of SEQ ID NO: 22 and the amino acid sequence of SEQ ID NO: 21.

Embodiment 33. The fusion protein construct according to Embodiment 32, wherein the amino acid sequence of SEQ ID NO: 22 is N terminal of the amino acid sequence of SEQ ID NO: 21.

Embodiment 34. The fusion protein construct according to any one of Embodiments 1 to 14, wherein the IL-15 module has, from N terminus to C terminus, the amino acid sequence of SEQ ID NO: 22 followed by 2, 3 or 4 repeats of the amino acid sequence of SEQ ID NO: 31, followed by the amino acid sequence of SEQ ID NO: 21.

Embodiment 35. The fusion protein construct according to any one of Embodiments 1 to 14, wherein the IL-15 module has the amino acid sequence of SEQ ID NO: 21 comprising the mutation lle67Glu.

Embodiment 36. The fusion protein construct according to any one of Embodiments 1 to 35, wherein the IL-15 module is fused to a C terminus of the antibody module.

Embodiment 37. The fusion protein construct according to any one of Embodiments 1 to 21, wherein the IL-15 module does not comprises an IL-15 receptor α chain or a fragment thereof, especially the extracellular domain of the IL-15 receptor α chain or the sushi domain of the IL-15 receptor α chain or a fragment thereof capable of binding to IL-15.

Embodiment 38. The fusion protein construct according to any one of Embodiments 1 to 37, wherein the fusion protein construct comprises two IL-15 modules, each fused to the C terminus of a different heavy chain of the antibody module.

Embodiment 39. The fusion protein construct according to Embodiment 38, wherein the heavy chains of the antibody module do not comprise a C terminal lysine residue.

Embodiment 40. The fusion protein construct according to Embodiment 38, wherein the heavy chains of the antibody module do not comprise the two C terminal residues glycine and lysine.

Embodiment 41. The fusion protein construct according to Embodiment 38, wherein the heavy chains of the antibody module do not comprise the three C terminal residues proline, glycine and lysine.

Embodiment 42. The fusion protein construct according to any one of Embodiments 38 to 41, wherein one or more of the three C terminal residues proline, glycine and lysine, if present, of the heavy chains of the antibody module are substituted, especially by leucine or alanine or serine.

Embodiment 43. The fusion protein construct according to any one of Embodiments 1 to 42, wherein the fusion protein construct comprises two IL-15 modules, each fused to the C terminus of a different light chain of the antibody module.

Embodiment 44. The fusion protein construct according to any one of Embodiments 1 to 42, wherein the fusion protein construct comprises two IL-15 modules, each fused to the N terminus of a different light chain of the antibody module.

Embodiment 45. The fusion protein construct according to any one of Embodiments 1 to 44, wherein the fusion protein construct comprises a peptide linker between the antibody module and the IL-15 module.

Embodiment 46. The fusion protein construct according to Embodiment 45, wherein the peptide linker comprises the amino acid sequence of SEQ ID NO: 31, in particular 2 or more, especially 2, 3 or 4, repeats of the amino acid sequence of SEQ ID NO: 31.

Embodiment 47. The fusion protein construct according to Embodiment 46, wherein the peptide linker consists of 2, 3 or 4 repeats of the amino acid sequence of SEQ ID NO: 31.

Embodiment 48. The fusion protein construct according to Embodiment 45, wherein the peptide linker comprises the amino acid sequence of SEQ ID NO: 32, in particular 2 or more, especially 3 or 6, repeats of the amino acid sequence of SEQ ID NO: 32.

Embodiment 49. The fusion protein construct according to Embodiment 48, wherein the peptide linker consists of 3 or 6 repeats of the amino acid sequence of SEQ ID NO: 32.

Embodiment 50. The fusion protein construct according to any one of Embodiments 45 to 49, wherein the peptide linker further comprises an additional N terminal proline, aspartate or alanine residue.

Embodiment 51. The fusion protein construct according to any one of Embodiments 1 to 44, wherein the fusion protein construct does not comprise a peptide linker between the antibody module and the IL-15 module.

Embodiment 52. The fusion protein construct according to any one of Embodiments 1 to 51, wherein the antibody module does not comprise an N-glycosylation site in the CH2 domain.

Embodiment 53. The fusion protein construct according to any one of Embodiments 1 to 51, wherein the antibody module comprises an N-glycosylation site in the CH2 domain of the antibody heavy chains.

Embodiment 54. The fusion protein construct according to Embodiment 53, wherein the antibody module has a glycosylation pattern in the CH2 domain of the antibody heavy chains, having a relative amount of glycans carrying a core fucose residue of at least 60% of the total amount of glycans attached to the CH2 domains of the antibody module in a composition.

Embodiment 55. The fusion protein construct according to Embodiment 53, wherein the antibody module has a glycosylation pattern in the CH2 domain of the antibody heavy chains, having a relative amount of glycans carrying a core fucose residue of 40% or less of the total amount of glycans attached to the CH2 domains of the antibody module in a composition.

Embodiment 56. The fusion protein construct according to any one of Embodiments 1 to 55, comprising a further agent conjugated thereto.

Embodiment 57. The fusion protein construct according to Embodiment 56, wherein the further agent is a polypeptide or protein which is fused to a polypeptide chain of the antibody module or to a polypeptide chain of the IL-15 module.

Embodiment 58. The fusion protein construct according to Embodiment 57, wherein the antibody module comprises two antibody heavy chains and two antibody light chains, wherein a IL-15 module is fused to the C terminus of each antibody light chain, and wherein a further agent being a polypeptide or protein is fused to the C terminus of each antibody heavy chain.

Embodiment 59. The fusion protein construct according to Embodiment 57, wherein the antibody module comprises two antibody heavy chains and two antibody light chains, wherein a IL-15 module is fused to the C terminus of each antibody heavy chain, and wherein a further agent being a polypeptide or protein is fused to the C terminus of each antibody light chain.

Embodiment 60. The fusion protein construct according to any one of Embodiments 56 to 59, wherein the further agent is selected from the group consisting of cytokines, chemokines, antibody modules, antigen binding fragments, enzymes and binding domains.

Embodiment 61. A nucleic acid encoding the fusion protein construct according to any one of Embodiments 1 to 60.

Embodiment 62. An expression cassette or vector comprising the nucleic acid according to Embodiment 61 and a promoter operatively connected with said nucleic acid.

Embodiment 63. A host cell comprising the nucleic acid according to Embodiment 61 or the expression cassette or vector according to Embodiment 62.

Embodiment 64. A pharmaceutical composition comprising the fusion protein construct according to any one of Embodiments 1 to 60 and one or more further components selected from the group consisting of solvents, diluents, and excipients.

Embodiment 65. The fusion protein construct according to any one of Embodiments 1 to 60 or the pharmaceutical composition according to Embodiment 64 for use in medicine.

Embodiment 66. The fusion protein construct according to any one of Embodiments 1 to 60 or the pharmaceutical composition according to Embodiment 58 for use in the treatment, prognosis, diagnosis and/or monitoring of diseases associated with abnormal cell growth such as cancer; infections such as bacterial, viral, fungal or parasitic infections; and diseases associated with a reduce immune activity such as immunodeficiencies.

Embodiment 67. The fusion protein construct or pharmaceutical composition according to Embodiment 66 for use in the treatment of cancer, wherein the cancer is selected from the group consisting of cancer of the breast, colon, stomach, liver, pancreas, kidney, blood, lung, endometrium, thyroid and ovary.

Embodiment 68. The fusion protein construct or pharmaceutical composition according to Embodiment 66 for use in the treatment of infections, wherein the infection is selected from the group consisting of bacterial infections, viral infections, fungal infections and parasitic infections.

Embodiment 69. A fusion protein construct, comprising
(i) an anti-MUC1 antibody module, the antibody module comprising two antibody heavy chains and two antibody light chains, each heavy chain comprising a VH domain, a CH1 domain, a hinge region, a CH2 domain and a CH3 domain, and each light chain comprising a VL domain and a CL domain; and
(ii) two IL-15 modules, each comprising human IL-15.

Embodiment 70. The fusion protein construct according to Embodiment 69, wherein the antibody heavy chains each comprise a set of heavy chain CDR sequences with CDR-H1 having the amino acid sequence of SEQ ID NO: 1, CDR-H2 having the amino acid sequence of SEQ ID NO: 33 and CDR-H3 having the amino acid sequence of SEQ ID NO: 5.

Embodiment 71. The fusion protein construct according to Embodiment 70, wherein each VH domain of the anti-MUC1 antibody module comprises an amino acid sequence which is at least 80% identical, especially 100% identical, to SEQ ID NO: 34.

Embodiment 72. The fusion protein construct according to Embodiment 70 or 71, wherein the amino acid residue at position 8 of SEQ ID NO: 33 and position 57 of SEQ ID NO: 34 is any amino acid residue except asparagine, especially glutamine or alanine.

Embodiment 73. The fusion protein construct according to Embodiment 69, wherein the antibody heavy chains each comprise a set of heavy chain CDR sequences with CDR-H1 having the amino acid sequence of SEQ ID NO: 1, CDR-H2 having the amino acid sequence of SEQ ID NO: 3 and CDR-H3 having the amino acid sequence of SEQ ID NO: 5.

Embodiment 74. The fusion protein construct according to Embodiment 73, wherein each VH domain of the anti-MUC1 antibody module comprises an amino acid sequence which is at least 80% identical, especially 100% identical, to SEQ ID NO: 8 or 9.

Embodiment 75. The fusion protein construct according to any one of Embodiments 70 to 74, wherein the antibody light chains each comprise a set of light chain CDR sequences with CDR-L1 having the amino acid sequence of SEQ ID NO: 10, CDR-L2 having the amino acid sequence of SEQ ID NO: 12 and CDR-L3 having the amino acid sequence of SEQ ID NO: 14.

Embodiment 76. The fusion protein construct according to Embodiment 75, wherein each VL domain of the anti-MUC1 antibody module comprises an amino acid sequence which is at least 80% identical, especially 100% identical, to SEQ ID NO: 17 or 18, especially 18.

Embodiment 77. The fusion protein construct according to Embodiment 69, wherein the antibody heavy chains each comprise a set of heavy chain CDR sequences with CDR-H1 having the amino acid sequence of SEQ ID NO: 2, CDR-H2 having the amino acid sequence of SEQ ID NO: 4 and CDR-H3 having the amino acid sequence of SEQ ID NO: 6.

Embodiment 78. The fusion protein construct according to Embodiment 77, wherein each VH domain of the anti-MUC1 antibody module comprises an amino acid sequence which is at least 80% identical, especially 100% identical, to SEQ ID NO: 7.

Embodiment 79. The fusion protein construct according to Embodiment 77 or 78, wherein the antibody light chains each comprise a set of light chain CDR sequences with CDR-L1 having the amino acid sequence of SEQ ID NO: 11, CDR-L2 having the amino acid sequence of SEQ ID NO: 13 and CDR-L3 having the amino acid sequence of SEQ ID NO: 15.

Embodiment 80. The fusion protein construct according to Embodiment 79, wherein each VL domain of the anti-MUC1 antibody module comprises an amino acid sequence which is at least 80% identical, especially 100% identical, to SEQ ID NO: 16.

Embodiment 81. The fusion protein construct according to Embodiment 69, wherein each VH domain of the anti-MUC1 antibody module comprises an amino acid sequence which is at least 80% identical, especially 100% identical, to SEQ ID NO: 9, and a set of heavy chain CDR sequences with CDR-H1 having the amino acid sequence of SEQ ID NO: 1, CDR-H2 having the amino acid sequence of SEQ ID NO: 3 and CDR-H3 having the amino acid sequence of SEQ ID NO: 5.

Embodiment 82. The fusion protein construct according to Embodiment 69, wherein each VH domain of the anti-MUC1 antibody module comprises an amino acid sequence which is at least 80% identical, especially 100% identical, to SEQ ID NO: 9, and a set of heavy chain CDR sequences with CDR-H1 having the amino acid sequence of SEQ ID NO: 1, CDR-H2 having the amino acid sequence of SEQ ID NO: 3 and CDR-H3 having the amino acid sequence of SEQ ID NO: 5, wherein the antibody molecule comprises a mutation to the effect that the amino acid residue at position 8 of SEQ ID NO: 3 which corresponds to the amino acid residue at position 57 of SEQ ID NO: 9 is substituted by any other amino acid residue except Asn, especially by Gln or Ala, in particular by Gln.

Embodiment 83. The fusion protein construct according to Embodiment 81 or 82, wherein each VL domain of the anti-MUC1 antibody module comprises an amino acid sequence which is at least 80% identical, especially 100% identical, to SEQ ID NO: 18, and a set of light chain CDR sequences with CDR-L1 having the amino acid sequence of SEQ ID NO: 10, CDR-L2 having the amino acid sequence of SEQ ID NO: 12 and CDR-L3 having the amino acid sequence of SEQ ID NO: 14.

Embodiment 84. The fusion protein construct according to any one of Embodiments 69 to 83, wherein the IL-15 module comprises an amino acid sequence which is at least 80% identical, especially 100% identical, to SEQ ID NO: 21.

Embodiment 85. The fusion protein construct according to Embodiment 84, wherein the IL-15 module further comprises an amino acid sequence which is at least 80% identical, especially 100% identical, to SEQ ID NO: 22.

Embodiment 86. The fusion protein construct according to Embodiment 85, comprising a peptide linker comprising 2, 3 or 4 repeats of the amino acid sequence GGGGS (SEQ ID NO: 31) between the C terminus of the amino acid sequence which is at least 80% identical, especially 100% identical, to SEQ ID NO: 22 and the N terminus of the amino acid sequence which is at least 80% identical, especially 100% identical, to SEQ ID NO: 21.

Embodiment 87. The fusion protein construct according to any one of Embodiments 69 to 84, wherein the IL-15 module does not comprise an IL-15 receptor α chain or a fragment thereof, especially the extracellular domain of the IL-15 receptor α chain or the sushi domain of the IL-15 receptor α chain or a fragment thereof capable of binding to IL-15.

Embodiment 88. The fusion protein construct according to any one of Embodiments 69 to 87, wherein a peptide linker is present between the IL-15 modules and the antibody module.

Embodiment 89. The fusion protein construct according to any one of Embodiments 69 to 88, wherein the IL-15 modules are fused to C terminus of the heavy chains of the antibody module.

Embodiment 90. The fusion protein construct according to Embodiment 89, comprising a peptide linker comprising 4 repeats of the amino acid sequence GGGGS

(SEQ ID NO: 31) between the C terminus of the heavy chains of the antibody module and the N terminus of the IL-15 modules.

Embodiment 91. The fusion protein construct according to Embodiment 89, comprising a peptide linker comprising 3 repeats of the amino acid sequence GGGGS (SEQ ID NO: 31) between the C terminus of the heavy chains of the antibody module and the N terminus of the IL-15 modules.

Embodiment 92. The fusion protein construct according to Embodiment 89, comprising a peptide linker comprising 2 repeats of the amino acid sequence GGGGS (SEQ ID NO: 31) between the C terminus of the heavy chains of the antibody module and the N terminus of the IL-15 modules.

Embodiment 93. The fusion protein construct according to Embodiment 89, comprising a peptide linker comprising 3 repeats of the amino acid sequence PAPAP (SEQ ID NO: 32) between the C terminus of the heavy chains of the antibody module and the N terminus of the IL-15 modules.

Embodiment 94. The fusion protein construct according to Embodiment 89, comprising a peptide linker comprising 6 repeats of the amino acid sequence PAPAP (SEQ ID NO: 32) between the C terminus of the heavy chains of the antibody module and the N terminus of the IL-15 modules.

Embodiment 95. The fusion protein construct according to any one of Embodiments 90 to 94, wherein the peptide linker further comprises an additional N terminal proline, aspartate or alanine residue.

Embodiment 96. The fusion protein construct according to any one of Embodiments 89 to 95, wherein the heavy chains of the antibody module do not comprise a C terminal lysine residue.

Embodiment 97. The fusion protein construct according to any one of Embodiments 89 to 95, wherein the heavy chains of the antibody module do not comprise the two C terminal residues glycine and lysine.

Embodiment 98. The fusion protein construct according to any one of Embodiments 89 to 95, wherein the heavy chains of the antibody module do not comprise the three C terminal residues proline, glycine and lysine.

Embodiment 99. The fusion protein construct according to any one of Embodiments 89 to 98, wherein one or more of the three C terminal residues proline, glycine and lysine, if present, of the heavy chains of the antibody module are substituted with another amino acid residue, in particular with alanine or leucine.

Embodiment 100. The fusion protein construct according to any one of Embodiments 69 to 88, wherein the IL-15 modules are fused to C terminus of the light chains of the antibody module.

Embodiment 101. The fusion protein construct according to Embodiment 100, comprising a peptide linker comprising 2, 3 or 4 repeats of the amino acid sequence GGGGS (SEQ ID NO: 31) between the C terminus of the light chains of the antibody module and the N terminus of the IL-15 modules.

Embodiment 102. The fusion protein construct according to Embodiment 100, comprising a peptide linker comprising 3 or 6 repeats of the amino acid sequence PAPAP (SEQ ID NO: 32) between the C terminus of the light chains of the antibody module and the N terminus of the IL-15 modules.

Embodiment 103. The fusion protein construct according to any one of Embodiments 69 to 88, wherein the IL-15 modules are fused to N terminus of the light chains of the antibody module.

Embodiment 104. The fusion protein construct according to Embodiment 103, comprising a peptide linker comprising 2, 3 or 4 repeats of the amino acid sequence GGGGS (SEQ ID NO: 31) between the N terminus of the light chains of the antibody module and the C terminus of the IL-15 modules.

Embodiment 105. The fusion protein construct according to any one of Embodiments 69 to 99, wherein each IL-15 module comprises human IL-15 and is fused with its N terminus via a peptide linker to the C terminus of a different heavy chain.

Embodiment 106. The fusion protein construct according to any one of Embodiments 69 to 88, wherein each IL-15 module comprises human IL-15 and is fused with its N terminus via a peptide linker to the C terminus of a different light chain.

Embodiment 107. The fusion protein construct according to any one of Embodiments 69 to 88, wherein each IL-15 module comprises human IL-15 and is fused with its C terminus via a peptide linker to the N terminus of a different light chain.

Embodiment 108. The fusion protein construct according to any one of Embodiments 69 to 99, wherein each IL-15 module comprises human IL-15 and is fused with its N terminus via a peptide linker to the C terminus of a different heavy chain, wherein the heavy chains do not comprise a C terminal lysine residue.

Embodiment 109. The fusion protein construct according to any one of Embodiments 69 to 87, wherein each IL-15 module comprises human IL-15 and is fused with its N terminus directly to the C terminus of a different heavy chain, wherein the heavy chains do not comprise a C terminal lysine residue.

Embodiment 110. The fusion protein construct according to any one of Embodiments 69 to 84 and 87 to 109, wherein the IL-15 module consists of human IL-15.

Embodiment 111. The fusion protein construct according to Embodiment 110, wherein human IL-15 has the amino acid sequence of SEQ ID NO: 21.

Embodiment 112. The fusion protein construct according to any one of Embodiments 69 to 111, wherein the antibody module does not comprise an N-glycosylation site in the CH2 domain of each antibody heavy chains.

Embodiment 113. The fusion protein construct according to Embodiment 112, wherein the antibody module does not comprise an asparagine residue at the position in the heavy chain corresponding to position 297 according to the IMGT/Eu numbering system, in particular comprises an Ala297 mutation in the heavy chain.

Embodiment 114. The fusion protein construct according to any one of Embodiments 69 to 111, wherein the antibody module comprises an N-glycosylation site in the CH2 domain of each antibody heavy chains.

Embodiment 115. The fusion protein construct according to Embodiment 114, wherein the antibody module has a glycosylation pattern in the CH2 domain of the antibody heavy chains, wherein the relative amount of glycans carrying a core fucose residue is at least 60%, especially at least 65% or at least 70% of the total amount of glycans attached to the CH2 domains of the antibody module in a composition of the fusion protein construct.

Embodiment 116. The fusion protein construct according to Embodiment 114, wherein the antibody module has a glycosylation pattern in the CH2 domain of the antibody heavy chains, wherein the relative amount of glycans carrying a core fucose residue is 40% or less, especially 30% or less or 20% or less of the total amount of glycans attached to the CH2 domains of the antibody module in a composition of the fusion protein construct.

Embodiment 117. The fusion protein construct according to any one of Embodiments 69 to 116 for use in the treatment of diseases associated with abnormal cell growth such as cancer, infections such as bacterial, viral, fungal or parasitic infections and immunodeficiencies.

Embodiment 118. The fusion protein construct according to any one of Embodiments 69 to 116 for use in the treatment of ovarian cancer, breast cancer such as triple negative breast cancer, lung cancer or pancreatic cancer.

Embodiment 119. The fusion protein construct according to any one of Embodiments 1 to 60 and 69 to 116 for use in the treatment of cancer in combination with a bispecific antibody targeting MUC1 and CD3.

Embodiment 120. The fusion protein construct according to any one of Embodiments 1 to 60 and 69 to 116 for use in the treatment of cancer in combination with an antibody against PD-L1.

Embodiment 121. The fusion protein construct according to any one of Embodiments 1 to 60 and 69 to 116 for use in the treatment of cancer in combination with an antibody against EGFR, such as tomuzotuximab or cetuximab.

Embodiment 122. The fusion protein construct according to any one of Embodiments 1 to 60 and 69 to 116 for use in the treatment of cancer in combination with an antibody against CD40.

### FIGURES

FIG. 1 shows different fusion protein constructs comprising wildtype IL-15 (IL-15 wt), IL-15 with a mutation reducing receptor binding (IL-15mut) or a combination of the IL-15Rα sushi domain and IL-15 (IL-15sushi) attached to the C terminus of the heavy chain or the C or N terminus of the light chain of an anti-MUC1 antibody (αMUC1).

FIG. 2 illustrates the antigen binding characteristics of PM-IL15wt NA and PM-IL15wt to glycosylated and non-glycosylated MUC1 peptides as measure of tumor specificity analyzed by ELISA. αMUC1 without IL-15 (PankoMab) was used as control.

FIG. 3 shows binding of PM-IL-15 immunocytokines to the TA-MUC1 expressing tumor cell line T-47D as analyzed by flow cytometry. The αMUC1-IL-15 constructs were compared with similar fusion constructs with an antibody which does not bind the target cells (MOPC-IL-15wt/sushi). ααMUC1 without IL-15 (PankoMab) was used as reference.

FIG. 4 shows binding of PM-IL15 wt NA and PM-IL15 wt to tumor-cell expressed TA-MUC1 on PANC-1 analyzed by flow cytometry. αMUC1 without IL-15 (PankoMab) and irrelevant human IgG1 were used as positive and negative control, respectively.

FIG. 5 shows binding of PM-IL-15 immunocytokines to the IL-15 receptor domains IL-15Rα (A) and IL-2/IL-15Rβ (B). Binding was analyzed by ELISA. αMUC1 without IL-15 (PankoMab) was used as control.

FIG. 6 shows binding of PM-IL15wt NA and PM-IL15wt to IL15 receptor subunits IL15Rα and IL15Rβ analyzed by ELISA. αMUC1 without IL-15 (PankoMab) was used as control.

FIG. 7 shows binding of PM-IL-15 immunocytokines with and without a functional Fc part to Fc gamma receptor llla as analyzed by a competitive Alphascreen assay. αMUC1 without IL-15 (PankoMab) was used as control.

FIG. 8 shows induction of natural cytotoxicity by PM-IL-15 immunocytokines against the TA-MUC1 negative Jurkat cell line in presence of PBMC. Cytotoxicity was analyzed by Europium release assay after 5 h. αMUC1 without IL-15 (PankoMab) was used as control.

FIG. 9 shows immune cell mediated antibody-mediated cellular cytotoxicity (ADCC) against target cells initiated by the fusion protein constructs. PBMCs containing NK cells and T cells were incubated with different αMUC1-IL-15 constructs in the presence of MUC1⁺ T47D target cells. Specific lysis of the target cells depending on the concentration of the fusion protein construct was determined. αMUC1 without IL-15 was used as control.

FIG. 10 shows immune cell mediated ADCC against target cells initiated by the fusion protein constructs. PBMCs containing NK cells and T cells were incubated with different αMUC1-IL-15 constructs in the presence of MUC1⁺ Ovcar-3 target cells. Specific lysis of the target cells depending on the concentration of the fusion protein construct was determined. The αMUC1-IL-15 constructs were compared to equivalent untargeted control constructs (MOPC-IL-15wt/sushi). αMUC1 without IL-15 was used as control.

FIG. 11 shows induction of ADCC by PM-IL-15 immunocytokines against TA-MUC1 positive MCF-7 breast cancer cells in presence of PBMC. Cytotoxicity was analyzed by LDH release assay after 24 h. αMUC1 without IL-15 (PankoMab) was used as control.

FIG. 12 shows the induction of cytotoxicity against TA-MUC1 expressing CaOV-3 tumor cells by PM-IL15wt NA and PM-IL15wt. PBMC from different donors were used as effector cells. Killing was determined by LDH release assay.

FIG. 13 shows that PM-IL-15 immunocytokines induce immune cell infiltration into TA-MUC1 expressing 3D tumor spheroids mimicking the immunosuppressive tumor environment. Spheroids co-cultivated with PBMC and immunocytokines or PBS as buffer control for 2 days were analyzed by immunohistochemistry to determine the number of CD45 or CD3 positive immune cells within the tumor after treatment.

FIG. 14 illustrates the induction of immune cell infiltration into TA-MUC1 expressing 3D tumor spheroids by PM-IL-15wt and PM-IL-15-wt NA. Spheroids co-cultivated with PBMC and immunocytokines, αMUC1 without IL-15 (PankoMab) or PBS as buffer control for 2 days were analyzed by immunohistochemistry to determine the number of CD45 or CD8 positive immune cells within the tumor after treatment.

FIG. 15 shows activation of NK cells (A) and NKT cells (B) by the fusion protein constructs. PBMCs containing NK cells and NKT cells were incubated in the presence of αMUC1-IL-15 constructs. Activation of NK cells and NKT cells was determined by CD69 expression. αMUC1 without IL-15 (PankoMab) was used as control.

FIG. 16 shows proliferation of NK cells (A), NKT cells (B) and CD8⁺ T cells (C) by the fusion protein constructs. PBMCs containing NK cells, NKT cells and CD8⁺ T cells were incubated in the presence of αMUC1-IL-15 constructs. Proliferation of the immune cells was determined by the percentage of divided cells. αMUC1 without IL-15 (PankoMab) was used as control.

FIG. 17 demonstrates the stimulatory properties of PM-IL15wt NA and PM-IL15wt on different immune cell populations. Activation markers CD25 and CD69 on NK and CD4+ and CD8+ T cells were analyzed by flow cytometry. αMUC1 without IL-15 (PankoMab) and medium without the addition of antibody were used as controls.

FIG. 18 shows the activation of memory and effector T cell subsets including naïe CD4+ and CD8+ T cells by PM-IL15wt NA and PM-IL15wt as analyzed by detection of activation marker expression via flow cytometry. αMUC1 without IL-15 (PankoMab) and medium without the addition of antibody were used as controls.

FIG. 19 shows the induction of CD4+ and CD8+ T and NK cell proliferation by PM-IL15wt NA and PM-IL15wt analyzed by flow cytometry. αMUC1 without IL-15 (PankoMab) and medium without the addition of antibody were used as controls.

FIG. 20 shows induction of cytokine release by PBMC of healthy donors after incubation with the PM-IL-15 immunocytokines. Medium, αMUC1 without IL-15 (PankoMab) and OKT3 served as controls for no, only moderate or high cytokine release. Secretion of IFN-γ and GM-CSF was analyzed by electrochemiluminescence.

FIG. 21 shows the reactivation of NK and T cells by PM IL15wt NA and PM-IL15wt after previous treatment of these immune cells with the cytokine TGF-β to mimic the immunosuppressive conditions in the tumor microenvironment. Re-activation of NK and T cells after treatment with the immunocytokines or a medium control was determined by analyzing activation markers via flow cytometry.

FIG. 22 shows the chemotactic properties of PM IL15wt NA and PM-IL15wt on immune cell subset analyzed in a transwell-based chemotactic assay. The number of NK (A), NKT (B) and CD8+ T cells (C) migrating from the upper to the lower chamber containing the stimulating immunocytokines or untargeted IL-15 was determined by flow cytometry. Results are expressed as chemotactic index related to an untreated control.

FIG. 23 shows the circulation half-life of different fusion protein constructs. αMUC1-1L-15wt NA and αMUC1-IL-15sushi NA were injected into mice and the plasma concentration of these constructs was monitored for 8 days. The calculated circulation half-lifes of the constructs are shown.

FIG. 24 shows the effect of different fusion protein constructs on the number of T cells in a murine model. αMUC1-IL-15wt NA and αMUC1-IL-15sushi NA were injected into mice and blood samples were analyzed predose and 8d after injection. The number of CD8+ T cells in the blood of the mice was determined by staining PBMC for CD45, CD3, CD4 and CD8.

FIG. 25 shows in vivo pharmacokinetic (PK) behavior of PM-IL15wt NA and PM-IL15wt after single dose i.v. injection into C57BL/6 mice. Serum concentrations determined at the different time points by ELISA are plotted (A) as well as the calculated PK parameters terminal serum half-life (t_{1/2}) and area under the curve (AUC) (B) from groups of 3 mice.

FIG. 26 shows the in vivo pharmacodynamic (PD) effects in C57BL/6 mice after single dose i.v. administration of the immunocytokines PM-IL-15wt NA and PM-IL-15wt or PBS as buffer control. Treatment-induced PD effects were analyzed in the lymphoid organs spleen and inguinal lymph nodes (ingLN) by flow cytometry. The increase of total cells in these organs is shown in A+D, relative proportions of different immune cell populations are shown in B+E whereas the final selective expansion of CD8+ T cells, NK cells and NKT cells is shown in C+F.

FIG. 27 (A) shows the increase of the CD8+/CD4+ Treg ratio by single dose treatment with PM-IL-15wt NA and PM-IL-15wt in comparison to a PBS control as analyzed by flow cytometry in the same model as described for FIG. 26. (B) shows the influence of treatment on relative proportion of different T cell subsets in the CD8+ population. Expression of ICOS, NKG2D and CD122 (IL-2/15Rβ) on CD8+ T cells (C) and NK cells (D) in the spleen was determined after treatment by flow cytometry.

FIG. 28 shows concentrations of the cytokines TNF-α and IFN-γ determined by ELISA in serum samples of these mice after treatment with immunocytokines or PBS as buffer control.

FIG. 29 shows the long term PD effects of treatment with PM-IL-15wt and PM-IL-15wt NA on immune cells in the peripheral blood. Relative proportions of NK, CD8+ T cells, CD4+ T cells, NKT cells, granulocytes and monocytes were determined by flow cytometry prior (day 0) and after treatment with the immunocytokines (day 11).

FIG. 30 shows binding of different PM-IL-15wt constructs to tumor-cell expressed TA-MUC1 on ZR-75-1 analyzed by flow cytometry. αMUC1 without IL-15 (PankoMab) was used as positive control.

FIG. 31 shows binding of different PM-IL-15wt constructs to the IL-15Rα subunit (CD215) analyzed by ELISA.

FIG. 32 shows the proliferation of CTLL-2 and KHyG-1 mCD16 in response to PM-IL-15-CH34GS and -Cκ4GS compared to recombinant IL-15.

FIG. 33 demonstrates the stimulatory properties of PM-IL-15-CH34GS and -Ck4GS compared to recombinant IL-15 on different immune cell populations. The activation marker CD25 was analyzed on NK and CD8+ T cells by flow cytometry. Medium without the addition of antibody was used as control.

FIG. 34 shows the induction of cytotoxicity against TA-MUC1 expressing CaOV-3 tumor cells by PM-IL-15-CH34GS and Cκ4GS compared to recombinant IL-15. Tumor cell killing was determined by LDH release assay.

FIG. 35 shows in vivo pharmacokinetic (PK) behavior of PM-IL-15-CH34GS and -Cκ4GS after single dose i.v. injection into C57BL/6. Calculated PK parameters are shown (terminal serum half-life (t_{1/2}) and area under the curve (AUC)) from groups of 3 mice.

FIG. 36 shows the in vivo pharmacodynamic (PD) effects in C57BL/6 mice after single dose i.v. administration of the immunocytokines PM-IL-15-CH34GS and PM-IL-15-Cκ4GS. Relative proportions of NK and CD8+ T cells as well as CD122 (IL15Rβ subunit) expression on both cell subsets in the blood were determined by flow cytometry prior (day 0) and after treatment with the immunocytokines (day 11).

FIG. 37 shows the influence of treatment with PM-IL-15-CH34GS and -Cκ4GS i.v. and s.c. on the relative proportions of different effector and memory T cell subsets in the CD4+ and CD8+ T cell population in vivo.

FIG. 38 shows the therapeutic effect of PM-IL-15-CH34GS on the tumor volume and survival of mice engrafted with TA-MUC1 positive 4T1 mouse tumor cells.

FIG. 39 shows the synergistic effects that were found on immune cell activation when combining the immunocytokine PM-IL-15-CH34GS with the TA-MUC1 targeting T cell engaging bi-specific (PM-CD3) in presence of CaOV-3 target cells. Treatment-induced expression of the activation marker CD25 on CD4+ (A) and CD8+ (B) T cells was determined after 2 days by flow cytometry.

FIG. 40 shows the synergistic effects that were found on T cell proliferation when combining the immunocytokines PM-IL-15wt NA and PM-IL-15wt with the TA-MUC1 targeting T cell engaging bi-specific (PM-CD3) in presence of CaOV-3 target cells. Treatment-induced proliferation of CD4+ (A) and CD8+ (B) T cells was determined after 5 days by flow cytometry.

FIG. 41 shows the synergistic effects that were found on PBMC-mediated cytotoxicity against CaOV-3 tumor cells after combined treatment of the immunocytokines PM-IL-15wt NA or PM-IL-15wt with the TA-MUC1 targeting T cell engaging bi-specific (PM-CD3). Cytotoxicity was determined after 24 h by LDH release assay. (A) shows absolute specific lysis, (B) further underlines synergism after subtraction of the lysis induced by the immunocytokines themselves. (C) shows the effects after combined treatment with 1 or 5 μg/mL of the immunocytokines.

FIG. 42 shows the expression of PD-L1 on HSC-4 tumor cells and monocytes after incubation for 2 days with 20 nM PM-IL-15-CH34GS compared to the control.

FIG. 43 shows the synergistic effects that were found on PBMC-mediated cytotoxicity against HSC-4 tumor cells after combined treatment of PM-IL-15-CH34GS with the PD-L1 targeting antibody Bavencio®. Cytotoxicity was determined after 24 h by LDH release assay.

FIG. 44 shows the synergistic effects that were found on T cell activation in a mixed lymphocyte reaction after combined treatment of PM-IL-15-CH34GS with the PD-L1 targeting antibody Bavencio®.

FIG. 45 shows the synergistic effects that were found on PBMC-mediated cytotoxicity against HSC-4 tumor cells after combined treatment of PM-IL-15-CH34GS with the EGFR targeting antibodies Erbitux® and CetuGEX®. Cytotoxicity was determined after 24 h by LDH release assay.

FIG. 46 shows the expression of CD215 on NK, NKT, CD4+, and CD8+ T cells after treatment with an glyco-optimized anti-CD40 hIgG1 antibody.

### EXAMPLES

### Example 1

### Production of Fusion Protein Constructs Specifically Binding MUC1 and IL-15

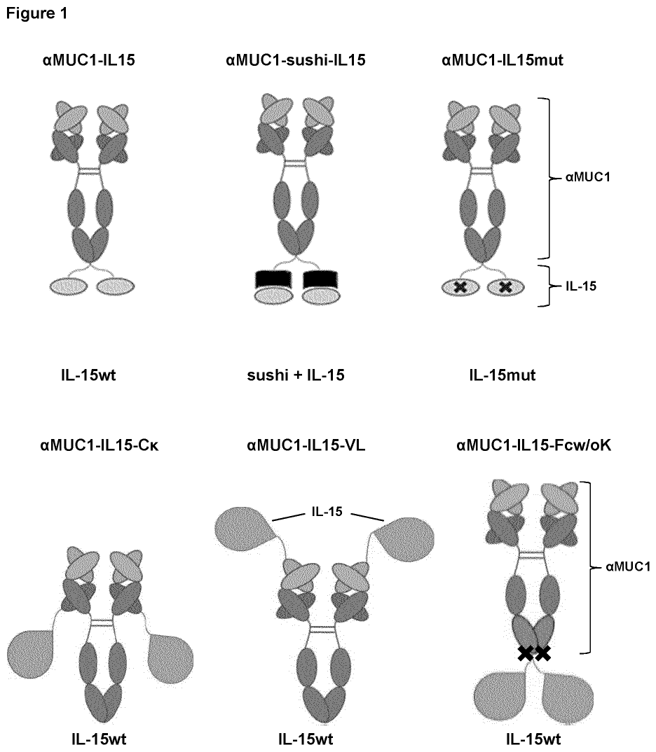

Fusion protein constructs were created that consist of a MUC1 specific binding part and an IL-15 function part. The MUC1 binding part is the humanized full-length IgG1 antibody molecule PankoMab (gatipotuzumab) with the typical antibody Y-shape. The anti-MUC1 antibody either comprises the natural glycosylation site in the CH2 domain (PM) or carries an N297A mutation in the heavy chain, abolishing glycosylation (PM NA). Without glycosylation in the CH2 domain, the antibody does not bind to Fcγ receptors and cannot induce ADCC (Fc silenced variant). IL-15 function is realized by fusion of full-length human IL-15 having the wildtype sequence (IL-15wt) or the mutation 167E (IL-15mut) which reduces receptor binding. In one construct, IL-15wt is accompanied by the sushi domain of the IL-15 receptor α-chain (IL-15sushi). The general structure of the constructs is shown in FIG. 1.

TABLE 1
Fusion protein constructs

| | PankoMab | IL-15 | IL-15Rα sushi |
|---|---|---|---|
| construct | glycosylation | sequence | domain |
| PM-IL-15wt | ✓ | wt | — |
| PM-IL-15mut | ✓ | I67E | — |
| PM-IL-15sushi | ✓ | wt | ✓ |
| PM-IL-15wt NA | — | wt | — |
| PM-IL-15mut NA | — | I67E | — |
| PM-IL-15sushi NA | — | wt | ✓ |

In these constructs, one IL-15 is fused to the C terminus of each antibody heavy chain via a (Gly₄Ser)₄ linker. The IL-15Rα sushi domain, when present, is fused between the

C terminus of the antibody heavy chain and the N terminus of IL-15, with (Gly₄Ser)₄ linker between the fusion partners.

TABLE 2
Further fusion protein constructs

| | position of | linker | presence of heavy |
|---|---|---|---|
| construct | IL-15 | sequence | chain C-term. Lys |
| PM-IL-15-CH34GS | HC C-term. | (GGGGS)₄ | ✓ |
| PM-IL-15-Cκ4GS | LC C-term. | (GGGGS)₄ | ✓ |
| PM-IL-15-Cκ1GS | LC C-term. | (GGGGS)₁ | ✓ |
| PM-IL-15-VL4GS | LC N-term. | (GGGGS)₄ | ✓ |
| PM-IL-15-CH34GS-oK | HC C-term. | (GGGGS)₄ | — |
| PM-IL-15-CH3oLi-oK | HC C-term. | without | — |
| | | linker | |

In these constructs, one IL-15wt is fused to the C terminus or N terminus of each antibody light chain via a (Gly₄Ser)₄ or (Gly₄Ser)₁ linker; or one IL-15 is fused to the C terminus of each antibody heavy chain via a (Gly₄Ser)₄ linker or directly without any linker, wherein the terminal lysine residue of the heavy chain was deleted. PankoMab is glycosylated in the CH2 domain. PM-IL-15-CH34GS corresponds to PM-IL-15wt of Table 1.

The constructs were expressed in the human myeloid leukemia derived cell line NM-H9D8 (DSM ACC2806), producing the constructs with a human glycosylation pattern having about 90% fucosylated glycans in the PankoMab CH2 domain. Additionally, the constructs may also be produced in the related cell line NM-H9D8-E6Q12 (DSM ACC2856), resulting in glycosylated constructs with a low amount of fucosylation of about 10% in the PankoMab CH2 domain of the wt constructs.

TABLE 3
Additional fusion protein constructs

| | | | C-terminal |
|---|---|---|---|
| | | | sequence of |
| | position of | linker | CH3 (EU |
| construct | IL-15 | sequence | numbering) |
| PM(N54Q)-4GS-IL15-CH3-Fcwt | HC C-term. | P(GGGGS)₄ | S₄₄₄P₄₄₅G₄₄₆K₄₄₇ |
| PM(N54Q)-3GS-IL15-CH3P-Fcwt | HC C-term. | P(GGGGS)₃ | S₄₄₄P₄₄₅G₄₄₆K₄₄₇ |
| PM(N54Q)-3xL1-IL15-CH3-Fcwt | HC C-term. | (PAPAP)₃ | S₄₄₄P₄₄₅G₄₄₆K₄₄₇ |
| PM(N54Q)-2GS-IL15-CH3D-Fcwt | HC C-term. | D(GGGGS)₂ | S₄₄₄P₄₄₅G₄₄₆K₄₄₇ |
| PM(N54Q)-3GS-IL15-CH3D-Fcwt | HC C-term. | D(GGGGS)₃ | S₄₄₄P₄₄₅G₄₄₆K₄₄₇ |
| PM(N54Q)-6xL1-IL15-CH3D-Fcwt | HC C-term. | D(PAPAP)₃ | S₄₄₄P₄₄₅G₄₄₆K₄₄₇ |
| PM(N54Q)-3GS-IL15-CH3KA-Fcwt | HC C-term. | (GGGGS)₃ | S₄₄₄P₄₄₅G₄₄₆A₄₄₇ |
| PM(N54Q)-2GS-IL15-CH3KA-Fcwt | HC C-term. | (GGGGS)₂ | S₄₄₄P₄₄₅G₄₄₆A₄₄₇ |
| PM(N54Q)-6xL1-IL15-CH3KA-Fcwt | HC C-term. | (PAPAP)₆ | S₄₄₄P₄₄₅G₄₄₆A₄₄₇ |
| PM(N54Q)-3xL1-IL15-CH3KA-Fcwt | HC C-term. | (PAPAP)₃ | S₄₄₄P₄₄₅G₄₄₆A₄₄₇ |
| PM(N54Q)-3GS-IL15-Ck-Fcwt | LC C-term. | (GGGGS)₃ | — |
| PM(N54Q)-3xL1-IL15-Ck-Fcwt | LC C-term. | (PAPAP)₃ | — |
| PM(N54Q)-6xL1-IL15-Ck-Fcwt | LC C-term. | (PAPAP)₆ | — |
| PM(N54Q)-2GS-IL15-CH3P-Fcwt | HC C-term. | P(GGGGS)₂ | S₄₄₄P₄₄₅G₄₄₆K₄₄₇ |
| PM(N54Q)-6xL1-IL15-CH3-Fcwt | HC C-term. | (PAPAP)₆ | S₄₄₄P₄₄₅G₄₄₆K₄₄₇ |
| PM(N54Q)-3xL1-IL15-CH3D-Fcwt | HC C-term. | D(PAPAP)₃ | S₄₄₄P₄₄₅G₄₄₆K₄₄₇ |
| PM(N54Q)-3GS-IL15-CH3oGK-Fcwt | HC C-term. | (GGGGS)₃ | S₄₄₄P₄₄₅ |
| PM(N54Q)-2GS-IL15-CH3oGK-Fcwt | HC C-term. | (GGGGS)₂ | S₄₄₄P₄₄₅ |
| PM(N54Q)-6xL1-IL15-CH3oGK-Fcwt | HC C-term. | (PAPAP)₆ | S₄₄₄P₄₄₅ |
| PM(N54Q)-3xL1-IL15-CH3oGK-Fcwt | HC C-term. | (PAPAP)₃ | S₄₄₄P₄₄₅ |
| PM(N54Q)-3GS-IL15-CH3GAoK-Fcwt | HC C-term. | (GGGGS)₃ | S₄₄₄P₄₄₅A₄₄₆ |
| PM(N54Q)-6xL1-IL15-CH3GAoK-Fcwt | HC C-term. | (PAPAP)₆ | S₄₄₄P₄₄₅A₄₄₆ |
| PM(N54Q)-3GS-IL15-CH3GSoK-Fcwt | HC C-term. | (GGGGS)₃ | S₄₄₄P₄₄₅S₄₄₆ |
| PM(N54Q)-6xL1-IL15-CH3GSoK-Fcwt | HC C-term. | (PAPAP)₆ | S₄₄₄P₄₄₅S₄₄₆ |
| PM(N54Q)-3GS-IL15-CH3PLoGK-Fcwt | HC C-term. | (GGGGS)₃ | S₄₄₄L₄₄₅ |
| PM(N54Q)-2GS-IL15-CH3PLoGK-Fcwt | HC C-term. | (GGGGS)₂ | S₄₄₄L₄₄₅ |
| PM(N54Q)-3xL1-IL15-CH3PLoGK-Fcwt | HC C-term. | (PAPAP)₃ | S₄₄₄L₄₄₅ |

The constructs were expressed in the human myeloid leukemia derived cell line NM-H9D8 (DSM ACC2806) and in the Chinese hamster ovary cell line CHO/dhFr—which lacks the enzyme dihydrofolate reductase (DHFR). Additionally, the constructs may also be produced in the NM-H9D8 related cell line NM-H9D8-E6Q12 (DSM ACC2856), resulting in glycosylated constructs with a low amount of fucosylation of about 10% in the PankoMab CH2 domain of the wt constructs.

### Analysis of Different PankoMab and IL-15 Variants

### Example 2

### Antigen Binding

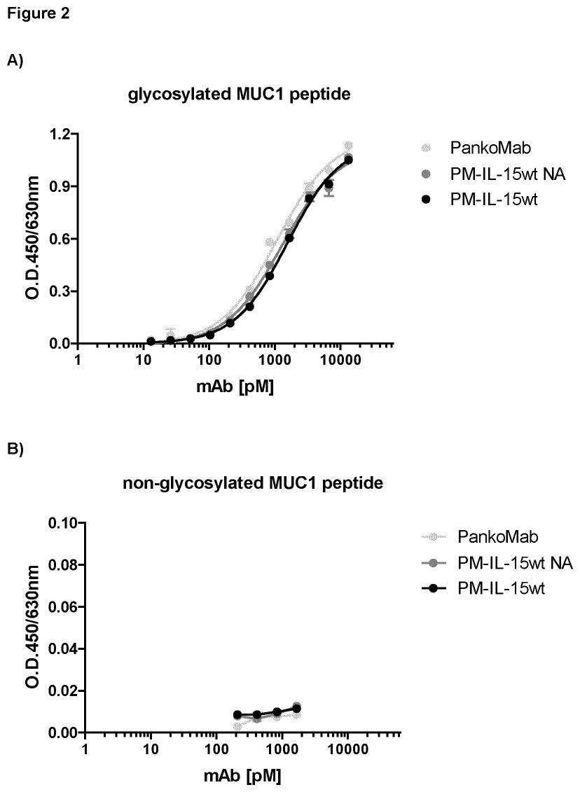

The antigen binding characteristics of PM-IL15wt NA and PM-IL15wt to glycosylated and non-glycosylated MUC1 peptides were compared to PankoMab using ELISA. Both PM-IL15wt NA and PM-IL15wt bind comparably to PankoMab to the glycosylated MUC1 peptides whereas there is no significant binding to the non-glycosylated MUC1 peptide (FIG. 2). This indicates adequate tumor specificity of both PM-IL15 constructs.

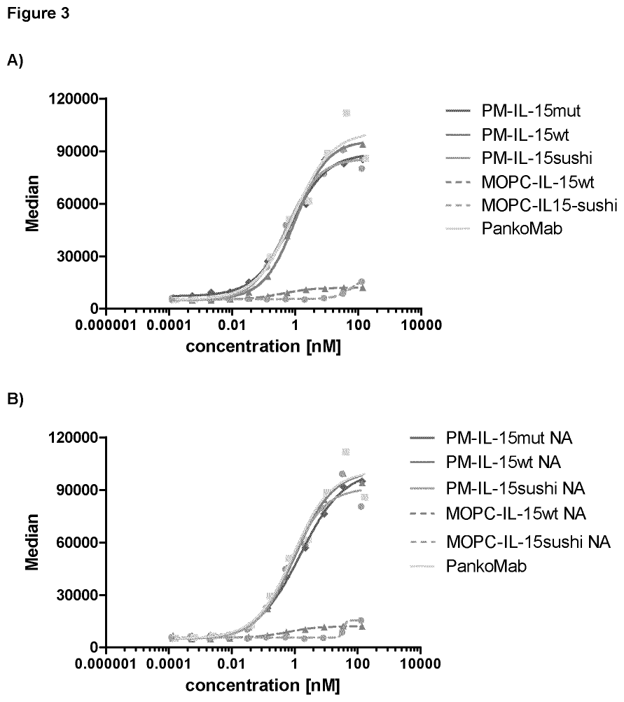

The binding properties of the different variants of PM-IL15 immunocytokines to cell surface TA-MUC1 were analyzed using the breast cancer cell line T-47D which strongly expresses TA-MUC1. Tumor cells were incubated with indicated antibodies in serial dilutions and bound antibodies were detected using a Phycoerythrin-conjugated goat anti-human IgG (heavy and light chain) antibody. Binding was analyzed by flow cytometry. All PM-IL15 immunocytokines show strong binding to T-47D cells irrespective of the IL15 variant attached or glycosylation of the Fc domain (Fc functional variants in FIG. 3A, Fc silenced variants in FIG. 3B). The binding properties (EC50, % maximum binding) were identical to PankoMab. Furthermore, the binding of PM-IL15 immunocytokines was highly specific to TA-MUC1 since no binding of the control constructs MOPC-IL15 (irrelevant Fab domain) was detected.

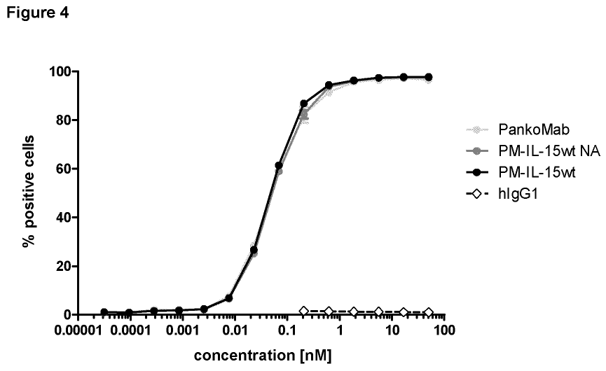

The binding of PM-IL15wt NA and PM-IL15wt to cell surface TA-MUC1 was additionally assessed by flow cytometry using the tumor cell line Panc-1 which strongly expresses TA-MUC1. The Panc-1 cells were incubated with different concentrations of PM-IL15wt and PM-IL15wt NA and compared to PankoMab. A human IgG control was included to control for background staining. Bound antibodies were detected using a Phycoerythrin-conjugated goat anti-human IgG (heavy and light chain) antibody. Both PM-IL15wt NA and PM-IL15wt show strong and specific binding to the TA-MUC1 expressing Panc-1 cells and the binding properties (EC50, % max) were completely identical to PankoMab (FIG. 4).

### Example 3

### IL-15 Receptor Binding

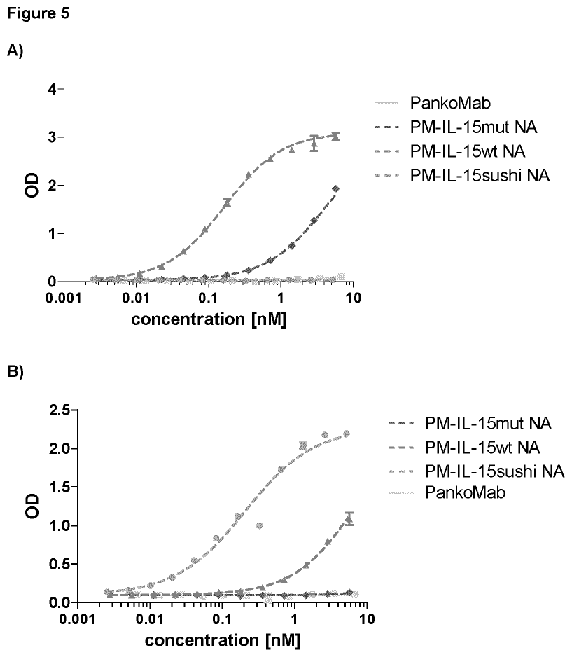

Binding properties to IL-15 receptor were analyzed exemplarily with the Fc silenced NA variants by ELISA. Either IL-15Rα or IL-15Rβ (IL-2rβ, CD122) was coated on 96-Well Maxisorp plates. PM-IL15 immunocytokines were incubated in serial dilutions and bound immunocytokines were detected by incubation with a peroxidase-labeled anti-human IgG F(ab′)2 fragment specific antibody. No binding to IL-15Rα is detectable for PM-IL15sushi NA and PM-IL15wt NA binds IL-15Rα with higher affinity compared to PM-IL15mut NA (FIG. 5A). Analysis of the binding to IL-15Rβ revealed that PM-IL15mut NA is not able to bind to the IL-15Rβ chain of the IL-15 receptor (FIG. 5B). The PM-IL15sushi NA variant showed a stronger binding to IL-15Rβ than PM-IL15wt NA.

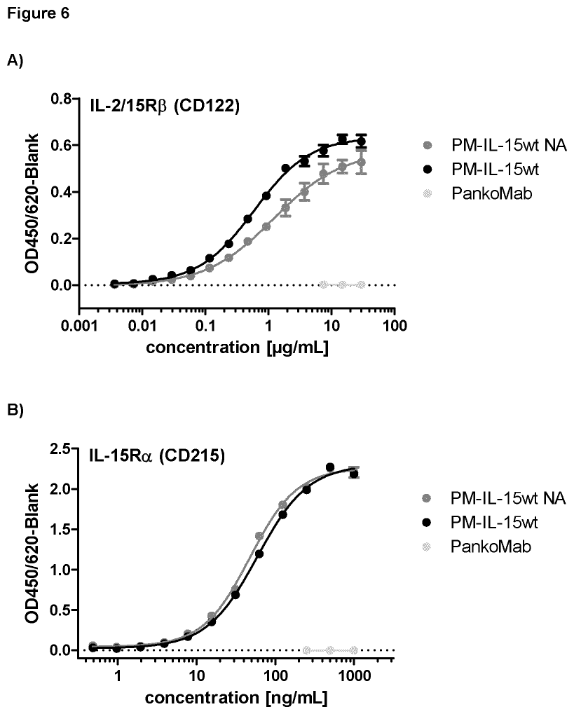

The capacity of PM-IL-15wt and PM-IL-15wt NA to bind to IL-15Rα (CD215) and IL-15Rβ (IL-2rβ, CD122) was compared by ELISA as described above after coating of either IL-15Rα or IL-15Rβ. Analysis of the binding to IL-15Rβ revealed that PM-IL15wt has a clearly stronger binding to CD122 than PM-IL15wt NA which could explain its higher activity on especially NK cells (FIG. 6A). The binding to IL-15Rα was comparable between both constructs (FIG. 6B).

### Example 4

### FcγRllla Binding

In order to characterize the binding of the antibody Fc domain of PM-IL15 immunocytokines to FcγRllla on a molecular level, we used a FcγR binding assay for FcγRllla (CD16a) based on the AlphaScreen® technology of PerkinElmer. The AlphaScreen® platform relies on simple bead-based technology of PerkinElmer and is a more efficient alternative to traditional ELISA.

His-tagged FcγRllla (Glycotope GmbH) is captured by Ni-chelate donor beads. The test PM-IL15 immunocytokines and rabbit-anti-mouse coupled acceptor beads compete for binding to Fcγllla. In case of interaction of FcγRllla with rabbit-anti-mouse acceptor beads, donor and acceptor beads come into close proximity which leads, upon laser excitation at 680 nm, to light emission by chemiluminescence. A maximum signal is achieved without a competitor. In case of competition, where a test antibody binds to FcγRllla, the maximum signal is reduced in a concentration-dependent manner. Chemiluminescence was quantified by measurement at 520-620 nm using an EnSpire 2300 multilabel reader (PerkinElmer). All results were expressed as the mean±standard deviation of duplicate samples. As a result, a concentration dependent sigmoidal dose-response curve was received, which is defined by top-plateau, bottom-plateau, slope, and EC50.

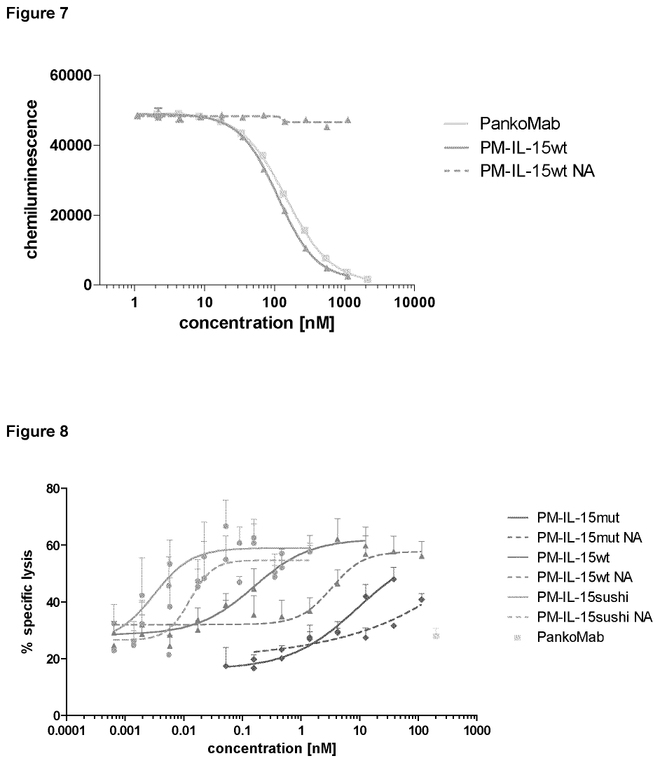

As shown in FIG. 7, PM-IL-15wt shows comparable FcγRllla binding to PankoMab-GEX whereas the Fc mutated N297A variant PM-IL-15wt NA does not show any FcγRllla binding.

### Example 5

### Induction of Natural Cytotoxicity

It is described that IL-15 is able to enhance natural cytotoxicity of immune cells. To test if the PM-IL-15 immunocytokines are able to induce natural cytotoxicity, the leukemic Jurkat T cell line was used as target cells and PBMC of a healthy donor as effector. Jurkat cells are described to be sensitive to natural cytotoxicity and do not express TA-MUC1. The natural cytotoxicity assay was performed as a Europium (Eu) release assay. Jurkat cells were loaded with europium by electroporation and seeded into assay plates. PBMC at an effector to target cell ratio of 80:1 and the dilutions of the test immuncytokines were added. All samples were analyzed in triplicates. For maximal europium release control (MR), target cells were lysed with TritonX-100. Basal europium release (BR) was measured from wells containing target cell supernatant. Finally, spontaneous europium release (SR) by target cells was addressed by controls containing target cells only. All controls were analyzed in sextuplicates. After 5 h of incubation, supernatants were harvested and released europium was determined after incubation with DELFIA Enhancement Solution and measurement on a Tecan Infinite F200 microplate reader at 340 nm extinction and 61 nm emission.

Specific cytotoxicity was calculated as:

% specific lysis=(experimental release−spontaneous release)/(maximal release −basal release)×100.

As shown in FIG. 8, all tested PM-IL-15 immunocytokines increase the natural cytotoxicity of immune cells against Jurkat T cells compared to PankoMab. The strength of the induction of natural cytotoxicity depends on the IL-15 module (IL-15sushi>IL-15wt>IL-15mut) and the variants with functional Fc domain have a higher potency compared to the Fc-silenced variants.

### Example 6

### ADCC Assays 1

Immune cell mediated antibody-dependent cell cytotoxicity (ADCC) is a main mechanism of anti-tumor antibodies. After tumor cell binding via TA-MUC1, the antibody construct activate NK cells and T cells by two different mechanisms. On the one hand, the Fc region of the antibody binds to Fcγ receptor Illa on the surface of the immune cells, and on the other hand the IL-15 portion of the constructs bind to interleukin receptors formed by the IL-2 receptor β-chain and the common γ-chain. The activated immune cells release cytotoxic granules containing perforin and granzymes that promote cell death of the TA-MUC1⁺ tumor cell.

The peripheral blood mononuclear cell (PBMC) ADCC assay was performed as an Europium (Eu) release assay. 3×10⁶ MUC-1 expressing T47D target cells with viabilities over 80% were harvested, washed twice in PBS and resuspended in 100 μL cold europium buffer. After 10 min incubation on ice, cells were electroporated with the Amaxa Nucleofector (Lonza). Electroporated cells were again incubated on ice for 10 min, before they were washed 6× in assay medium (RPM11640+5% (v/v) heat-inactivated FCS). Target cells were counted, diluted to 5×10⁴ cells/mL in assay medium and 100 μL/well added to the antibody dilutions or medium controls. 10× concentrated antibody dilutions were prepared in assay medium and 20 μL/well transferred into a 96-well round bottom plate. PBMCs were isolated and resuspended in assay medium to a density of 5×10⁶ cells/mL. 80 μL/well effector cells were transferred to the assay plates containing target cells and antibody dilutions or medium. All samples were analyzed in triplicates.

For maximal europium release control (MR), 100 μL/well target cells were supplemented with 80 μL/well medium and 20 μL/well 10% (v/v) TritonX-100. Basal europium release (BR) was measured from wells containing 100 μL assay medium and 100 μL target cell supernatant. Finally, spontaneous europium release (SR) by target cells was addressed my controls containing 100 μL assay medium and 100 μL target cells. All controls were analyzed in sextuplicates.

Plates were briefly centrifuged at 300×g and incubated for 4-5 h under standard cell culture conditions. To measure europium levels in the supernatant of assay wells, plates were centrifuged at 300×g for 5 min and 25 μL/well supernatant transferred to white 96-well flat bottom plates provided with 200 μL/well DELFIA Enhancement Solution. Plates were incubated in the dark for 10 min before fluorescence was measured with a Tecan Infinite F200 microplate reader at 340 nm extinction and 61 nm emission.

Specific cytotoxicity was calculated as:

% specific lysis=(experimental release−spontaneous release)/(maximal release−basal release)×100.

% spontaneous lysis=(spontaneous release−basal release)/(maximal release−basal release)×100.

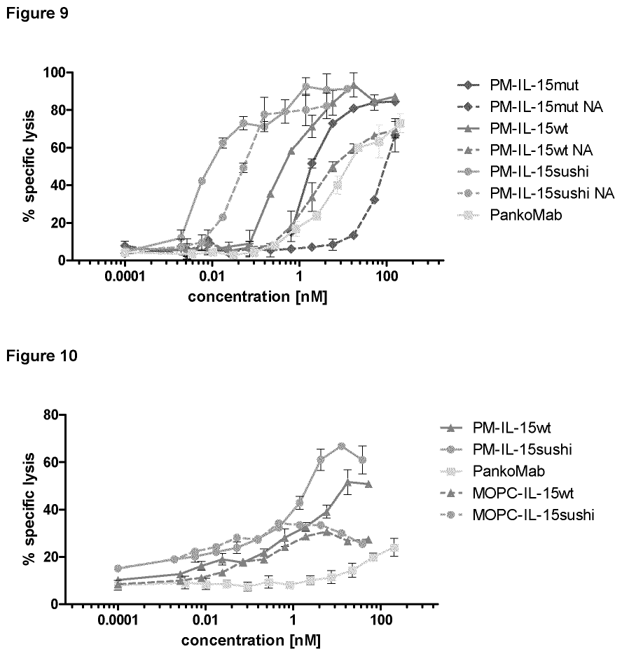

Using PBMCs as effector cells, the different fusion protein constructs showed target cell lysis of MUC-1 expressing tumor cell line T47D. The activities of the different constructs is demonstrated by the different EC50 values (concentration of the construct necessary to achieve half-maximal lysis). As expected the constructs comprising the IL-15Rα sushi domain in addition to IL-15 are more active than constructs with IL-15wt alone, which are more active than constructs with mutated IL-15 167E (see FIG. 9). The effect of the Fc region of the antibody is shown by comparing the PankoMab wt with the PankoMab NA constructs. Surprisingly, a very strong lytic activity could be observed even without immune cell activation via the antibody Fc region (PM-IL-15wt NA and PM-IL-15sushi NA). Except for the PankoMab NA construct with mutated IL-15, all constructs were more active than the naked PankoMab antibody.

The antigen binding of the fusion protein constructs is important for the ADCC effect, as demonstrated by comparison of the constructs with similar constructs with antibodies which do not bind tumor cells (MOPC). The MUC-1 expressing Ovcar-3 tumor cell line was used as target cells. MOPC constructs show a strongly reduced ADCC activity (see FIG. 10).

In a further ADCC assay, MCF-7 cells were used as target cells. TA-MUC1-positive MCF-7 tumor cells were grown for 24 h in assay plates before addition of unstimulated PBMC at an effector to target cell ratio of 10:1. Indicated concentrations of immunocytokines were added and tumor cell killing was assessed 24 h later by quantification of lactate dehydrogenase (LDH) released into cell supernatant (Cytotoxicity Detection Kit (LDH), Roche). Maximal release was achieved by incubation of target cells with triton-X-100 and antibody-independent cell death was measured in samples containing only target cells and PBMCs but no antibody.

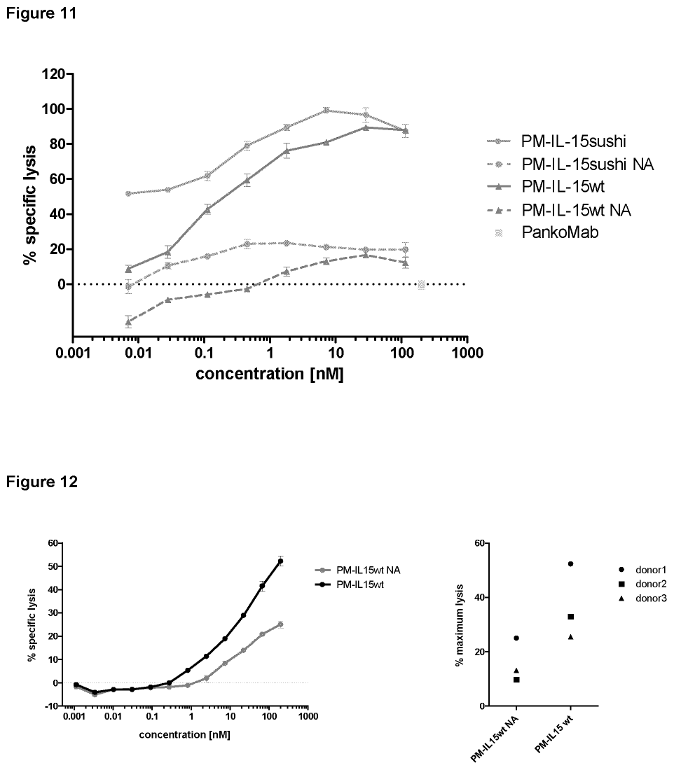

As expected, the constructs comprising the IL-15/IL-15Rα sushi domain are more active than constructs with IL-15wt alone (see FIG. 11). Surprisingly, a strong lytic activity could be observed even without immune cell activation via the antibody Fc region (PM-IL-15wt NA and PM-IL-15sushi NA). All tested immunocytokines were more active than the naked PankoMab antibody showing only moderate ADCC activity at the low E:T ratio of 10:1.

### Example 7

### ADCC Assays 2

Cytotoxicity against tumor cells is one of the main mechanisms which should be induced by immune therapeutics. The direction of IL-15 to tumor cells by using a TA-MUC1 targeted IL-15-based immuncytokine leads to the activation of immune cells and should further result in direct killing of tumor cells by granzyme B and perforin. To assess the cytotoxic potential of the PM-IL15 immunocytokines, TA-MUC1-positive CaOV-3 tumor cells were grown for 24 h in assay plates before addition of PM-IL15 immunocytokines and unstimulated PBMC at an effector to target cell ratio of 10:1. Tumor cell killing was assessed 24 h later by quantification of lactate dehydrogenase (LDH) released into cell supernatant (Cytotoxicity Detection Kit (LDH), Roche). Maximal release was achieved by incubation of target cells with triton-X-100 and antibody-independent cell death was measured in samples containing only target cells and PBMCs but no antibody. As shown in FIG. 12, both PM-IL15wt NA and PM-IL15wt induce effective lysis of tumor cells in a dose-dependent manner. Analysis of several donor PBMC revealed a higher potency of PM-IL15wt to induce tumor cell lysis (lower EC50, higher maximum lysis) compared to PM-IL15wt NA.

### Example 8

### Immune Cell Recruitment in a 3D Spheroid Model

A major advantage of tumor-targeted PM-IL15 immunocytokines is the mediation of local activation of immune cells at the tumor site to turn the immunosuppressive environment into a viable place of joint immune responses. But further, IL-15 is described to attract immune cells by its chemotactic properties. To analyze the potential of PM-IL15 immunocytokines to attract immune cells, we established a co-culture assay of PBMC with 3D tumor spheroids.

A MCF-7 breast cancer cell line was used which is enriched of cells with cancer stem cell (CSC) phenotype (termed _{MCF-}7_{CSC-enriched}). In contrast to the “classical” MCF-7 cell line, the CSC-enriched cell line shows a significantly increased proportion of the CD44+/CD24- and side population in normal adherent 2D culture and has the ability to form 3D spheres. 3D spheroids were generated by seeding TA-MUC1+ MCF-7_{CSC-enriched} cells in Corning Spheroid microplates followed by a 3 day incubation phase in a humidified atmosphere of 5% CO₂ at 37° C. Spheroid compaction and growth was confirmed by observation under a microscope. After spheroid formation at d3, human PBMCs of a healthy donor as well as 20 ng/ml PM-IL-15wt NA and PM-IL-15sushi NA were added and further incubated for 2 days. Infiltration of immune cells into the 3D spheroids was analyzed via immunohistochemistry. Therefore, spheroids were collected, washed and fixed with formalin. Fixed spheroids were then molded in HistoGel™ (Thermo Fisher), stained with tissue marking dye (Thermo Fisher), dehydrated, and embedded in paraffin. Staining was performed on 3-4 μm thick serial sections according to standard methods using antibodies against CD3 and CD45. Bound primary antibodies were detected by using peroxidase-coupled detection antibodies and liquid diaminobenzidine (DAB) as substrate. Nuclei were counterstained with hematoxylin.

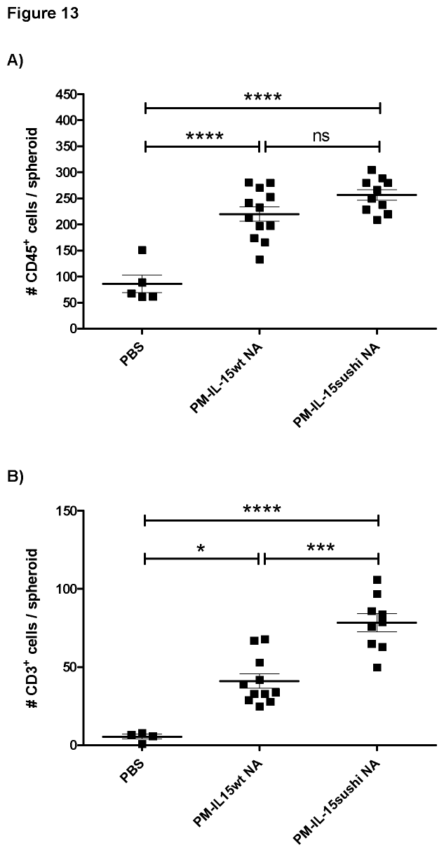

As shown in FIG. 13, addition of PM-IL-15wt NA and PM-IL-15sushi NA lead to a significant increase of CD45+ immune and CD3+ T cells within the spheroids. While there was no difference between PM-IL-15wt NA and PM-IL-15sushi NA in their potency to increase CD45+ immune cell infiltration, the PM-IL-15sushi NA hat a higher potency to recruit CD3+ T cells.

Furthermore, the potential of PM-IL-15wt and PM-IL-15-wt NA to attract immune cells were compared in the co-culture assay of PBMC with 3D tumor spheroids (method as described above).

After spheroid formation at d3, human PBMCs of a healthy donor as well as indicated amounts of PM-IL-15 immunocytokines or PankoMab were added and further incubated for 2 days. Infiltration of immune cells into the 3D spheroids was analyzed after staining of paraffin-embedded spheroids with CD8 and CD45. Bound primary antibodies were detected by using peroxidase-coupled detection antibodies and liquid diaminobenzidine (DAB) as substrate. Nuclei were counterstained with hematoxylin.

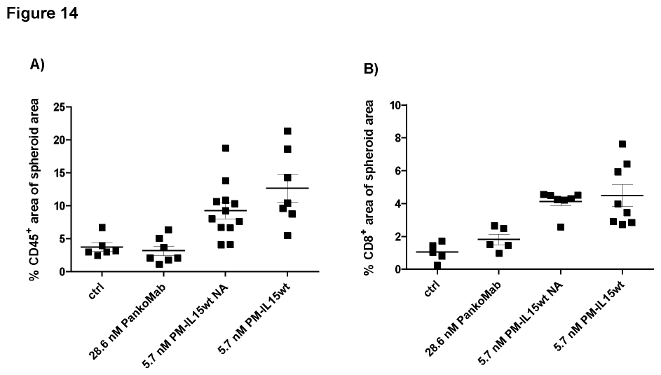

As shown in FIG. 14, addition of PM-IL-15wt and PM-IL-15wt NA lead to a significant increase of CD45+ immune and CD8+ T cells within the spheroids while there was no effect using PankoMab. There were no differences between PM-IL-15wt and PM-IL-15wt NA.

### Example 9

### Immune Cell Activation and Proliferation

To investigate activation of immune cells by the fusion protein constructs, expression of the activation markers CD69 after 48 h was analyzed by flow cytometry on NK cells and NKT cells. For this purpose human PBMCs from healthy donors were incubated with different fusion protein constructs at the indicated concentrations for 48 h. After 48 h PBMC's were harvested and stained with fluorescence labelled, αCD4, αCD8, αCD25, αCD69, αCD56, αCD14, and αCD19 antibodies, respectively. To solely analyze viable cells DAPI (Sigma-Aldrich) was used. Cells were analyzed in an Attune NxT (Thermo Fisher) flow cytometer. Besides immune cell activation another mechanism of action of the PankoMab-IL-15 constructs is the induction of NK and T cell proliferation. To measure immune cell proliferation PBMCs from healthy donors were labeled with CellTrace™ Violet (Thermo Fisher) and incubated with different fusion protein constructs for 5 days. If immune cells proliferate the CellTrace™ dye is diluted for each generation of proliferating cells. After 5 days PBMC's were harvested and stained with fluorescence labelled αCD4, αCD8, αCD56, αCD14, and αCD19 antibodies, respectively. To solely analyze viable cells 7-AAD (Calbiochem) was used. Cells were analyzed in an Attune NxT (Thermo Fisher) flow cytometer.

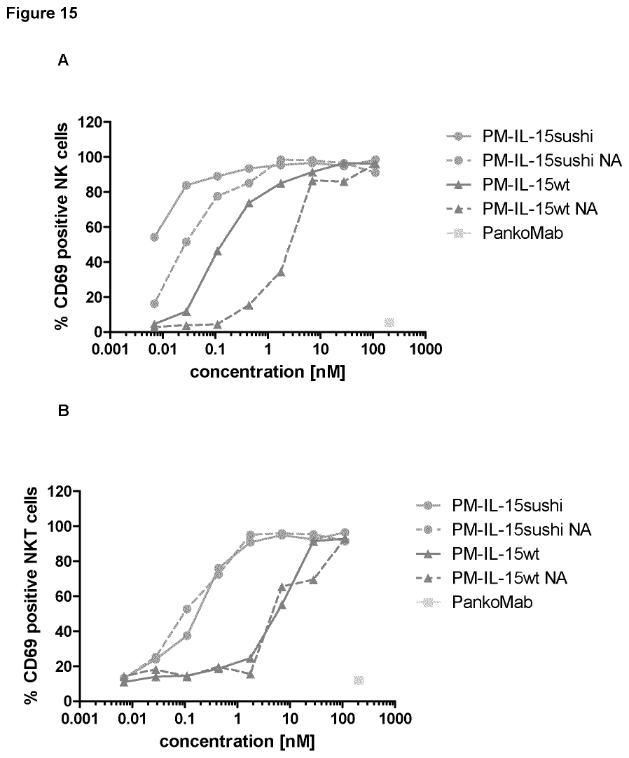

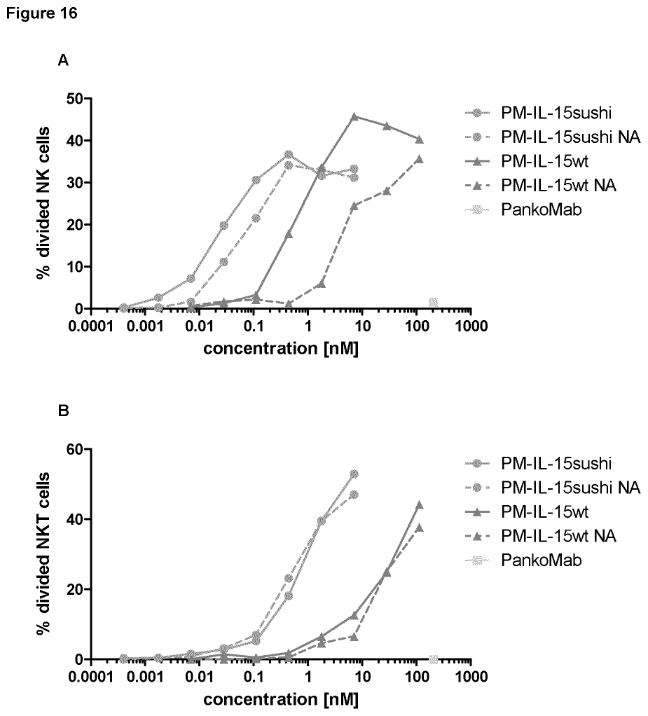

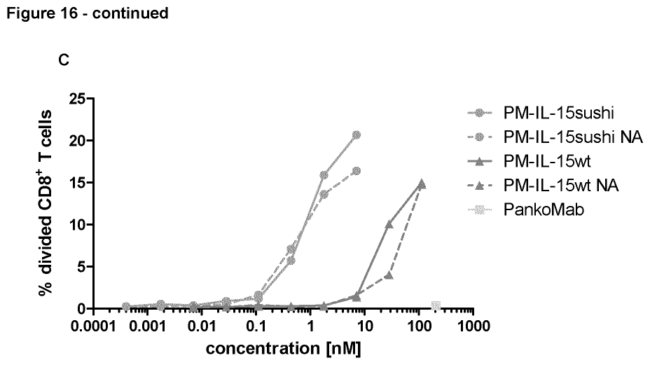

The results demonstrate that all fusion protein constructs are able to induce NK and NKT cell activation and NK, NKT and CD8⁺ T cell proliferation (see FIGS. 15 and 16). The strength of the induction of activation and proliferation depends on the IL-15 module (with IL-15sushi>IL-15wt>IL-15mut) and, for NK cells, on the glycosylation of the antibody Fc part (with glycosylated >non-glycosylated).

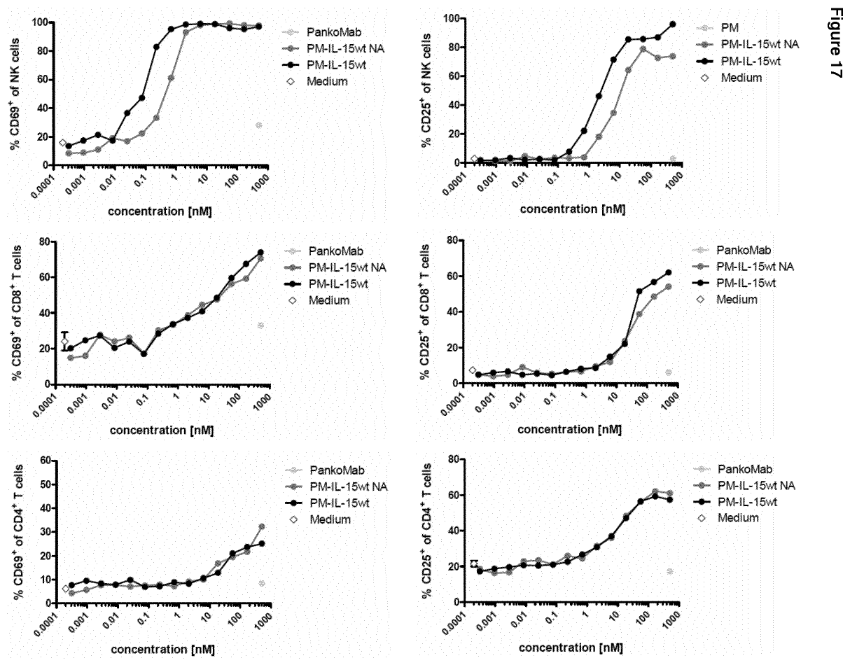

The immune stimulatory properties of PM-IL15 immunocytokines with and without Fc glycosylation (Fc wt and Fc silenced (NA) variants) was also investigated in detail. The activation of NK cells, CD8+ T cells and CD4+ T cells was analyzed after incubating PBMC for 5 days with the indicated molecules. Stimulated PBMC were stained with fluorescence labelled αCD3, αCD4, αCD8, αCD14, αCD19, αCD25, αCD45RA, αCD56, αCD69, and αCD197 antibodies. Dead cells were excluded by addition of DAPI before analysis. As shown in FIG. 17, PM-IL15wt NA and PM-IL15wt did induce expression of CD25 and CD69 on NK cells, CD4+ T cells and CD8+ T cells whereas PankoMab was not able to activate these cell subsets in this assay setup. Interestingly, the construct PM-IL15wt which is able to engage FcγR showed a higher potency to activate NK cells than PM-IL15wt NA which is unable to trigger FcγR activity. However, the expression of CD25 and CD69 on T cells induced by PM-IL15wt NA and PM-IL15wt was identical between both constructs.

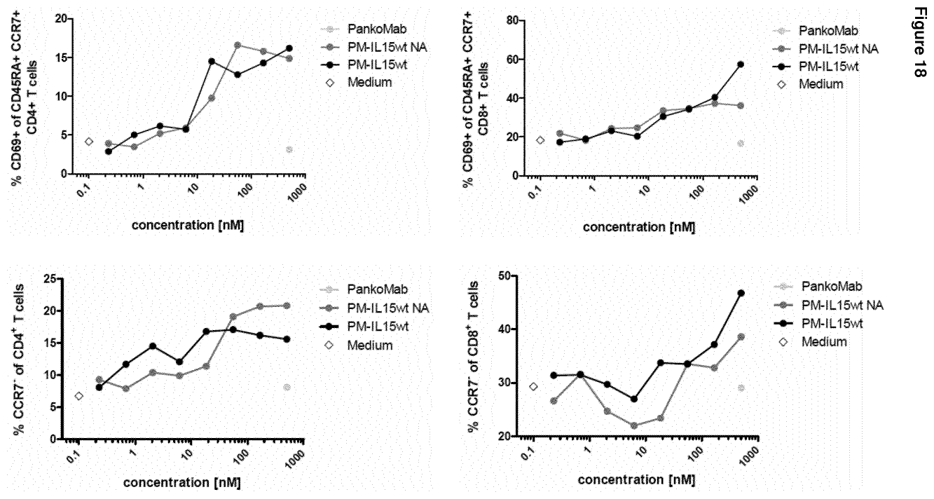

Interestingly, the analysis of memory and effector T cell subsets revealed that PM-IL15wt NA and PM-IL15wt were not only able to activate the classical effector populations but also naïve (CD45RA+ and CCR7+) CD4+ and CD8+ T cells as shown by the induction of CD69 on this particular cell subsets (FIG. 18). Furthermore, incubation with both PM-IL15wt NA and PM-IL15wt resulted in an increase of CCR7-effector T cells indicating that PM-IL15 immunocytokines are able to potentiate effector cell populations.

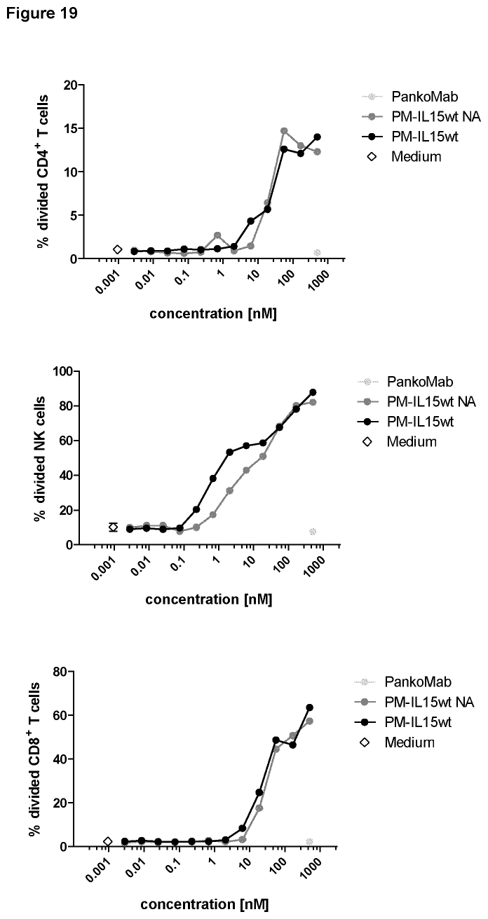

A full activation of NK and T cells results in robust proliferation. To assess the capacity of PM-IL15wt NA and PM-IL15wt to mediate proliferation, CellTrace™ Violet (Thermo Fisher)-labelled PBMC were incubated for 5 days with the indicated molecules. Stimulated PBMC were stained with fluorescence labelled αCD3, αCD4, αCD8, αCD14, αCD19, αCD45 and αCD56 antibodies. Dead cells were excluded by staining with 7-AAD (Sigma-Aldrich) before analysis by flow cytometry. Both PM-IL15wt NA and PM-IL15wt induced proliferation of NK cells, CD4+ T cells and CD8+ T cells while PankoMab alone had no effects (FIG. 19). Similar to the results of the immune cell activation assay, there was no difference in potency between PM-IL15wt NA and PM-IL15wt to induce T cell proliferation. However, PM-IL15wt reproducibly induced NK cell proliferation at lower concentrations than PM-IL15wt NA.

### Example 10

### Cytokine Release

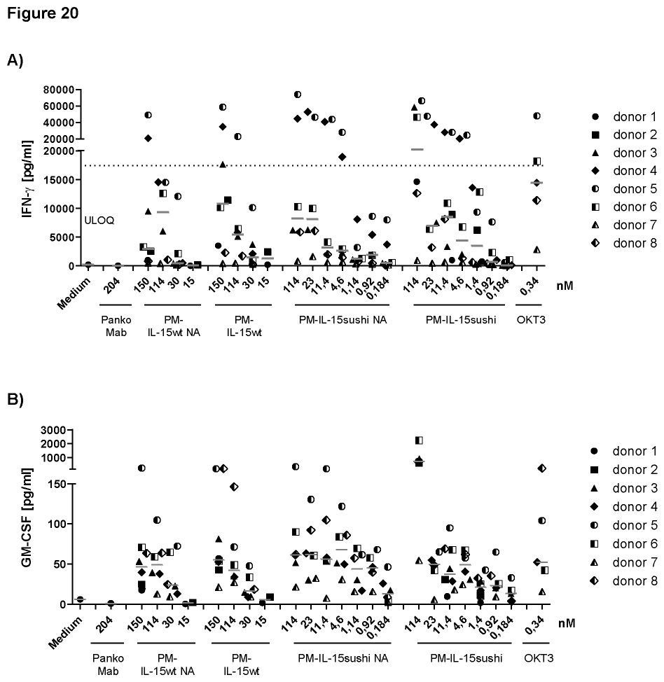

IL-15 is a potent cytokine and potential cytokine release mediated by IL-15 treatment is an issue which should be considered in preclinical studies. Especially the secretion of IFN-γ, GM-CSF and MIP1-α by immune cells is described after IL-15 stimulation. PBMC of eight healthy donors were incubated for 72 h with the indicated PM-IL-15 immunocytokines added to the solution phase of the assay well. Supernatants were analyzed using the UPLEX assay platform (MSD). Shown is the secretion of IFN-γ (FIG. 20A) and GM-CSF (FIG. 20B).

As expected, all tested PM-IL-15 immunocytokines induced a cytokine release and the constructs comprising the 1L-15/1L-15Rα sushi domain induced a higher secretion of IFN-γ (FIG. 20A) and GM-CSF (FIG. 20B) than constructs with IL-15wt alone. Surprisingly, there was no apparent influence of the Fc domains on the release of the tested cytokines.

### Example 11

### Activation of Immunosuppressed Cells

The microenvironment in solid tumors is generally highly suppressive, which is one of the main problems for quite a number of immune therapeutics to get implemented. To investigate if PM-1L15 immunocytokines have the ability to overcome a suppressive environment and activate suppressed immune cells, PBMC of healthy donors were incubated with 50ng/ml of the immunosuppressive cytokine TGF-β. After 48 h, PM-IL-15wt and PM-IL-15wt NA were added at equimolar concentrations (572 nM) and the activation of NK cells, CD8+ T cells and CD4+ T cells was analyzed after a further incubation of 5 days. PBMC were stained with fluorescence labelled αCD3, αCD4, αCD8, αCD25, αCD56 and αCD69. Dead cells were excluded by addition of DAPI before analysis.

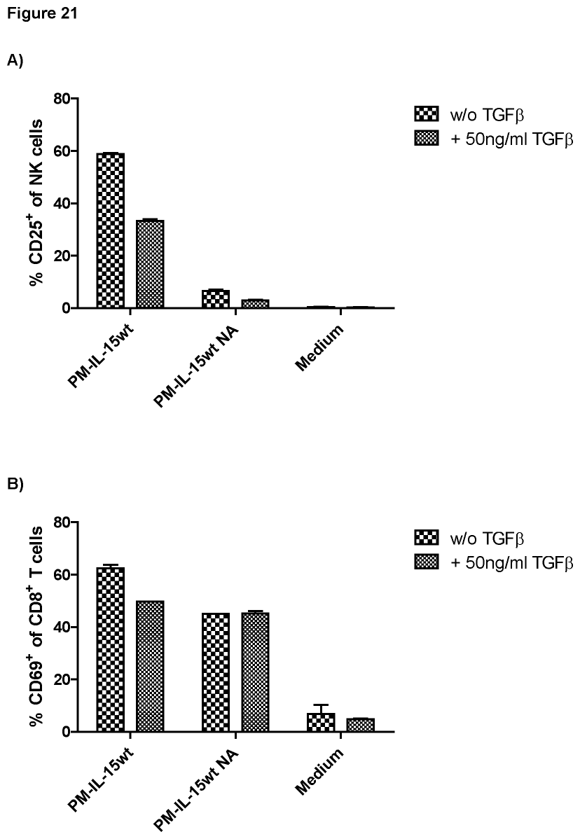

Activation of NK cells (CD25) is shown in FIG. 21A and of CD8+ T cells (CD69) in FIG. 21B. As expected and described earlier, PM-IL-15wt and PM-IL-15wt NA comparably activate unsuppressed T cells whereas NK cells are stronger activated by PM-IL-15wt than PM-IL-15wt NA. Interestingly, both PM-IL-15 immunocytokines were also able to activate immune cells suppressed by TGF-β. Immune suppression by TGF-β was visible on all analyzed cell subsets since the expression of CD25 and CD69 was reduced after treatment with TGF-β. Again, PM-IL-15wt and PM-IL-15wt NA showed similar potency to activate T cells and the PM-IL-15wt construct with functional Fc domain had a higher potency to stimulate suppressed NK cells compared to the Fc silenced variant PM-IL-15wt NA.

### Example 12

### Chemotaxis

IL-15 is described to attract immune cells by its chemotactic properties. To confirm that PM-IL-15 immunocytokines still act chemotactic, a classical chemotaxis assay was set up. Healthy PBMC were placed into the upper chamber of a 96-well Transwell system (5 μm pore size polycarbonate membrane, Corning Costar). The lower chamber was filled with medium to which PM-IL-15 immunocytokines were added. After incubation in 5% CO₂ for 4 h at 37° C., the number of migrated immune cells was determined by counting cells in the lower chamber using a flow cytometer. Prior to analysis, cells were stained with fluorescence labelled αCD3, αCD4, αCD8, αCD14, αCD19, αCD56, and DAPI.

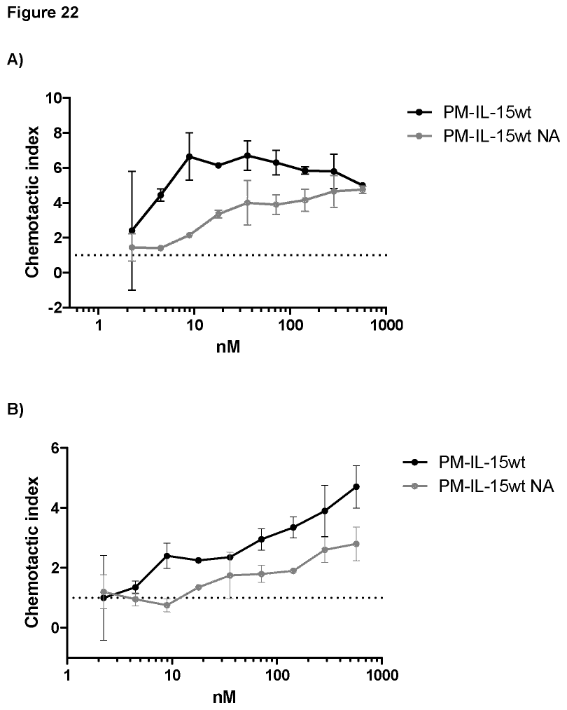

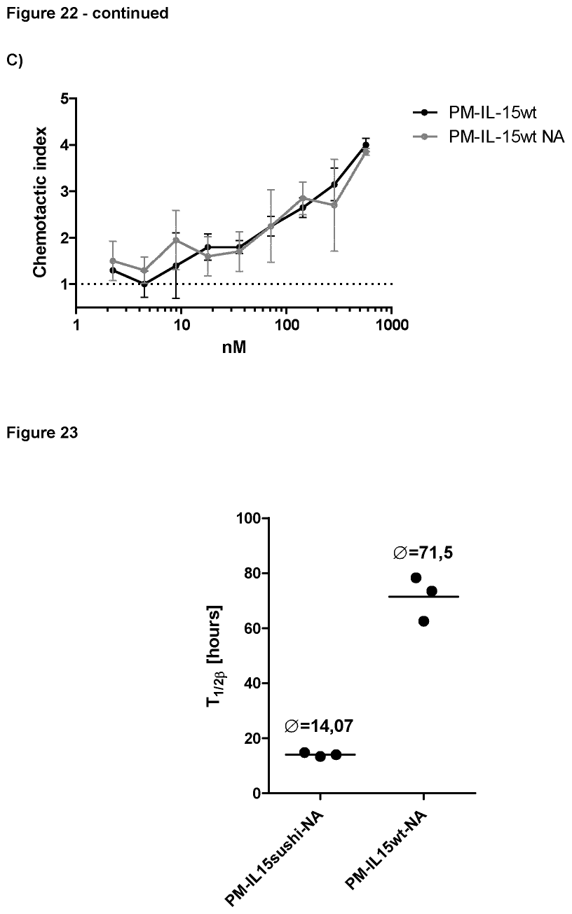

Shown is the migration of NK cells (FIG. 22A), NKT cells (FIG. 22B) and CD8+ T cells (FIG. 22C) towards the indicated PM-IL-15 immunocytokines relative to control wells (chemotactic index) without addition of any stimulus. Both PM-IL-15wt and PM-IL-15wt NA had a high potential to attract NK, NKT and CD8+ T cells. Interestingly, the variant PM-IL-15wt with functional Fc domain was more potent to induce the migration of NK cells than PM-IL-15wt NA while there was no clear difference regarding NKT and CD8+ T cells.

### Example 13

### Pharmacokinetics In Vivo

To analyze the pharmacokinetics of PM-IL-15sushi NA and PM-IL-15wt NA, C57BL/6 mice were injected i.v. with 200 pmol antibody. Serum was collected 5min, 1 h, 6 h, 1 d, 2 d, 3 d, 4 d, 5 d and 8 d after injection. In order to determine antibody titers of serum samples, Maxisorp F96 ELISA plates were coated with 2 μg/ml mouse anti-human Igκ light chain antibody in 0.1 M carbonate buffer pH 9.6 overnight. Unspecific binding was blocked with 5% (v/v) bovine serum albumin (BSA) in PBS (BSA/PBS). Serum samples and the standard were diluted in 1% (v/v) BSA/PBS. After sample incubation, horseradish peroxidase (HRP)-conjugated goat anti-human (IgG, Fcγ fragment specific) antibody was used at a dilution of 1:30.000 in 1% (v/v) BSA/PBS. For color development, TMB One Component HRP Microwell Substrate was added to the ELISA plate. The reaction was stopped with 1.25 M sulphuric acid and signals were measured at 450 nm and 620 nm using a Tecan Infinite F200 microplate reader.

Furthermore, blood samples were analyzed predose and 8d after injection. PBMC were stained with fluorophore-conjugated anti-mouse CD45, CD3, CD4, CD8 and cells were analyzed on an Attune® NxT Acoustic Focusing Cytometer.

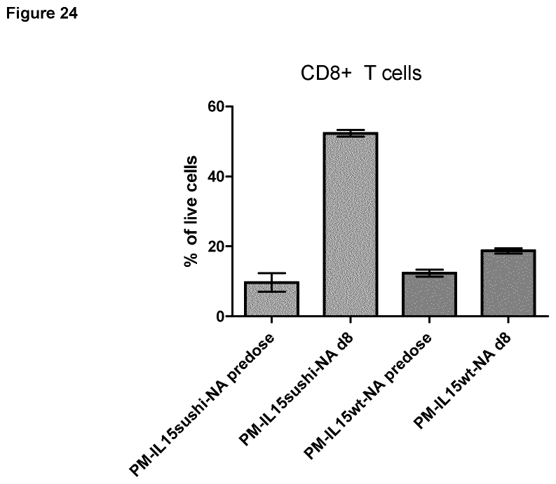

The results demonstrate a shorter half-life of PM-IL-15sushi NA compared to PM-IL-15wt NA (see FIG. 23). Further, treatment with both constructs leads to an increase of CD8+ T cells, the effect being more pronounced using PM-IL-15sushi NA (see FIG. 24).

To investigate if the FcγR-binding domain of PM-IL-15wt impacts its pharmacokinetic profile compared to PM-IL-15wt NA, C57BL/6 mice were injected i.v. with 2 mg/kg of the constructs (n=3). Serum was collected 5min, 6 h, 1 d, 2 d, 4 d, 7 d and 11 d after injection and antibody titers were determined by ELISA as described above.

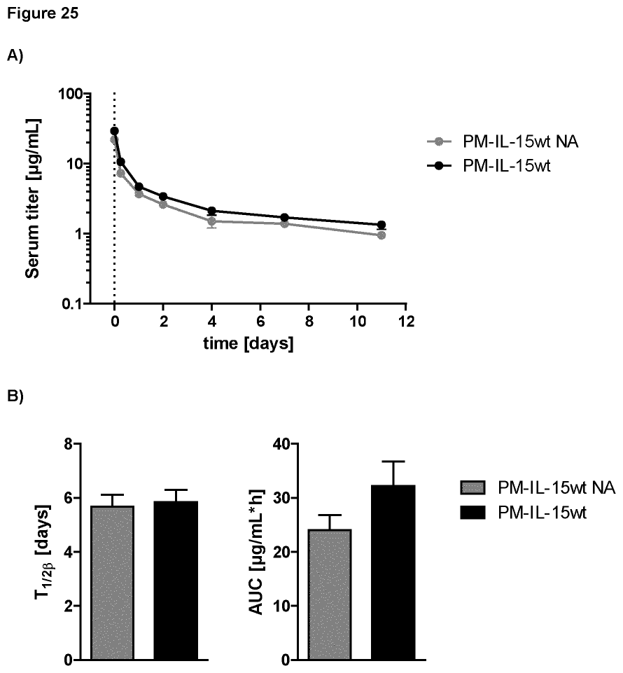

As shown in FIG. 25, PM-IL-15wt and PM-IL-15wt NA exhibit an identical t_{1/2} in C57BL/6 mice and a similar total exposition (AUC).

### Example 14

### Pharmacodynamics In Vivo

The pharmacodynamic effects of PM-IL-15 immunocytokines were further investigated in vivo. Mice (C57BL/6, n=3) were injected i.v. with either 2 mg/kg of PM-IL-15wt NA or PM-IL-15wt and were sacrificed on day 3 to collect blood, serum, inguinal lymph nodes (ingLN) and spleen. Serum was investigated to analyze effects on cytokine secretion and immune cells from blood and lymphoid organs were characterized for phenotype and frequencies of immune cell subsets.

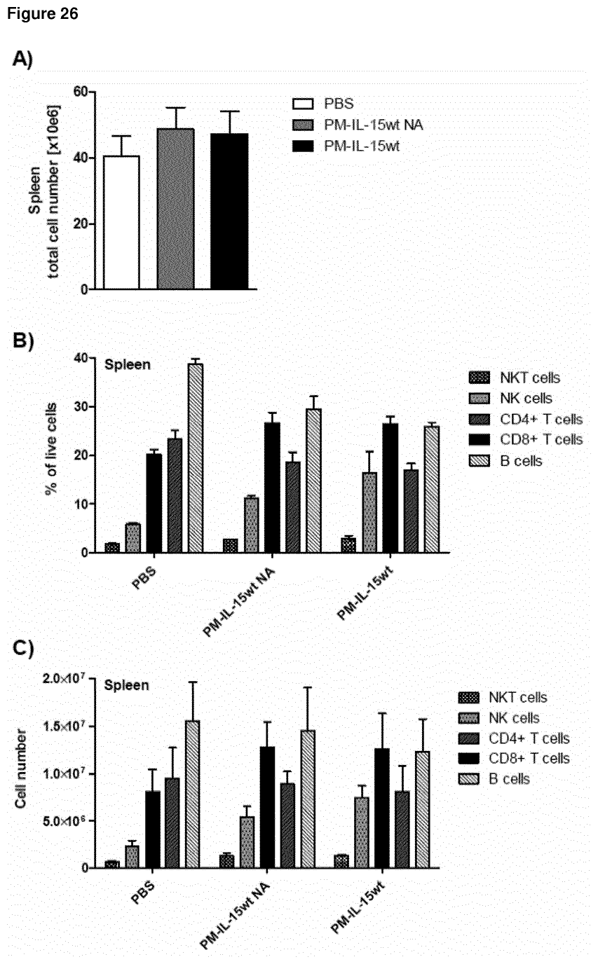

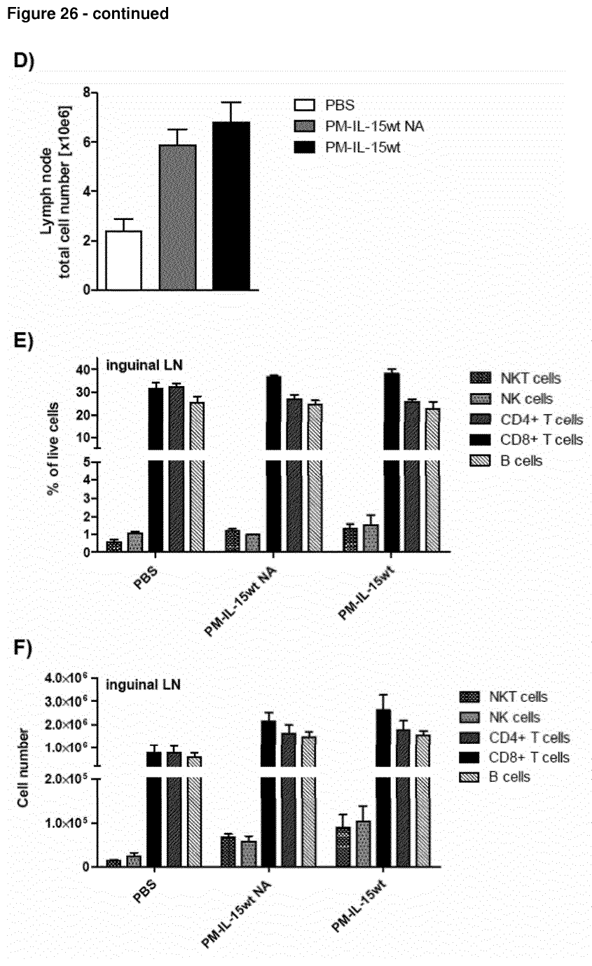

Treatment with PM-IL-15 immunocytokines for 3 days lead to an increase of the total number of cells within the spleen (FIG. 26A) and inguinal lymph nodes (FIG. 26D). Further, we observed a relative increase of CD8+ T cells, NK cells and NKT cells while CD4+ T cells and B cells rather decreased (FIG. 26B, E). Ultimately, this resulted in a selective expansion of CD8+ T cells, NK cells and NKT cells in spleen and ingLN (FIG. 26C, F) while the total cell number of B cells and CD4+ T cells were not (spleen) or only slightly (ingLN) affected. Generally, there were no differences between PM-IL-15wt NA and PM-IL-15wt except that PM-IL-15wt had a higher potency to stimulate NK cell expansion.

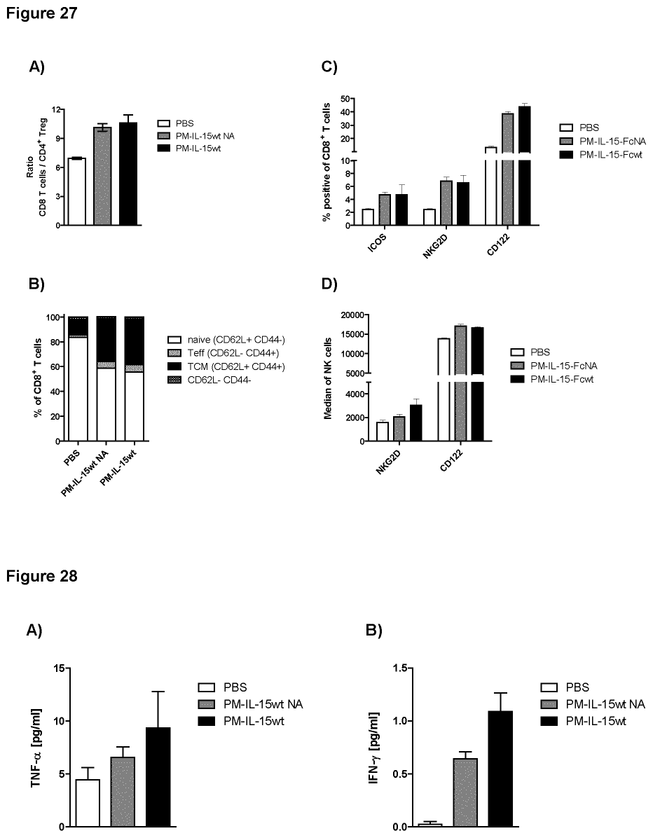

The selective expansion of CD8⁺ T cells induced by PM-IL-15 treatment for 3 days changed the ratio of CD8⁺ T cells to CD4⁺ regulatory T cells (Treg) in the spleen from initially 7:1 to 10:1 increasing the dominance of CD8+ T cells (FIG. 27A). Within the CD8+ T cell population, treatment with PM-IL-15wt and PM-IL-15wt NA lead to a relative reduction of naïve cells and concurrently to an increase of cells with central memory (TCM) and effector (Teff) phenotype (FIG. 27B). There were no differences between PM-IL-15wt and PM-IL-15wt NA. Similar effects were observed in ingLN (not shown).

The phenotypic characterization of mouse immune cells revealed that stimulation with PM-IL-15 immunocytokines for 3 days leads to the upregulation of ICOS, NKG2D and surprisingly also CD122 (IL-2/15Rβ) on CD8+ T cells (FIG. 27C). Similarly, NKG2D and CD122 were upregulated on NK cells (FIG. 27D). Both effects were visible in spleen (shown here) and ing LN (not shown). We observed no differences between PM-IL-15wt and PM-IL-15wt NA.

Serum of the mice was further analyzed on day 3 for cytokine content. Injection of PM-IL-15wt and PM-IL-15wt NA increased the cytokine level of TNF-αand IFN-γwith PM-IL-15wt inducing slightly stronger secretion of both cytokines compared to PM-IL-15wt NA (FIG. 28).

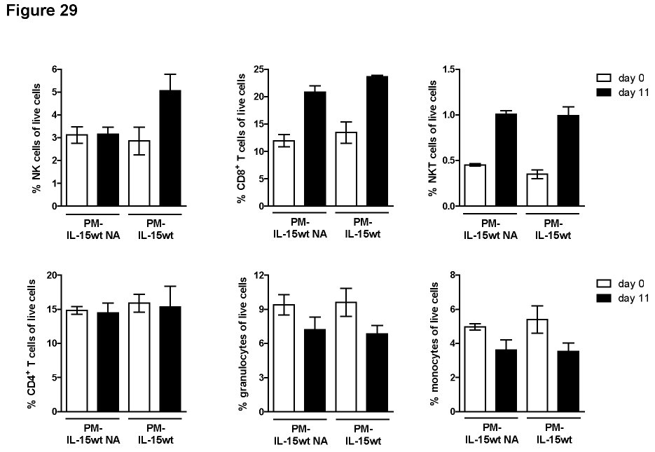

Finally, long term effects of PM-IL-15 immunocytokines were analyzed in vivo by analyzing immune cells in the blood on d11. A higher frequency of CD8+ T cells and NKT cells could still be observe in the blood after injection of PM-IL-15wt and PM-IL-15wt NA while a higher NK cell frequency was only observed with PM-IL15wt (FIG. 29). The frequency of CD4+ T cells was not affected and the percentages of granulocytes and monocytes were rather reduced by the treatment. This shows that a single injection of PM-IL-15 immunocytokines is able to induce long term effects on CD8+ T cells, NK cells and NKT cells.

### analysis of Different PM-IL-15 Construct Designs

### Example 15

### Antigen Binding

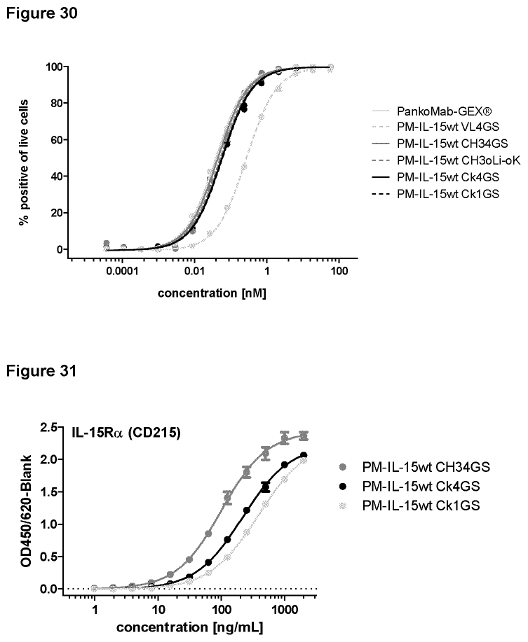

The binding properties of the different variants of PM-IL15 immunocytokines to cell surface TA-MUC1 were analyzed using the breast cancer cell line ZR-75-1 which strongly expresses TA-MUC1. Tumor cells were incubated with indicated antibodies in serial dilutions and bound antibodies were detected using a Phycoerythrin-conjugated goat anti-human IgG (heavy and light chain) antibody. Binding was analyzed by flow cytometry. Attachment of IL-15 to the OK or CH3 domain of the antibody (constructs CH34GS, CH3oLi-oK, Cκ4GS, COGS) did not influence the binding properties to TA-MUC1 when compared to the parental antibody PankoMab. Attachment of IL-15 to the VL region of the antibody (construct VL4GS) reduced the ability to bind to TA-MUC1 (FIG. 30).

### Example 16

### IL-15 Receptor Binding

Binding properties of different PM-IL-15wt constructs to IL-15 receptor subunits were analyzed by ELISA. IL-15Rα (CD215) was coated on 96-Well Maxisorp plates. PM-IL15 immunocytokines were incubated in serial dilutions and bound immunocytokines were detected by incubation with a peroxidase-labeled anti-human IgG F(ab′)2 fragment specific antibody. All tested constructs were able to bind to CD215 (FIG. 31). PM-IL-15-CH34GS showed superior binding to CD215 compared to the light chain fusion constructs PM-IL-15-Cκ4GS and PM-IL-15-Cκ1GS.

### Example 17

### Induction of Cell Proliferation

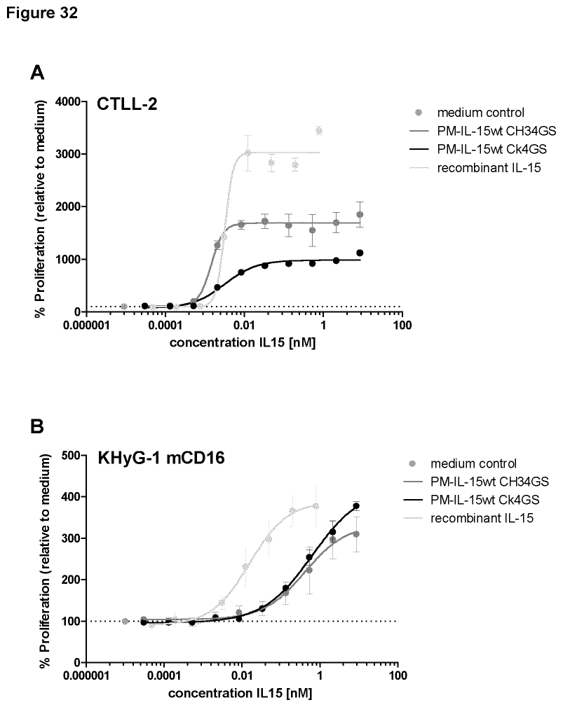

IL-15 is important for the survival of NK cells and memory CD8+ T cells and several cell lines of NK or T cell origin exist that are equally dependent on this cytokine for proliferation. The murine CTLL-2 T cell line is routinely used to test the biological activity of recombinant IL-15 by proliferation assay and also the natural killer cell leukemia cell line KHYG-1 mCD16 (KHYG-1 transfected with mouse CD16) responds to IL-15 with proliferation. These two cell lines were used to test the biological activity of IL-15 fused to either the CH3- or Cκ-domain of the PM-IL15 constructs in comparison to recombinant IL-15 (Miltenyi). The recombinant IL-15 had a higher potency to induce proliferation of CTLL-2 (FIG. 32A) and KHYG-1 mCD16 (FIG. 32B) cells compared to PM-IL-15-CH34GS and PM-IL-15-Cκ4GS when normalized to the molar concentration of applied of IL-15. Further, while PM-IL-15-CH34GS and PM-IL-15-Cκ4GS had an equal activity to induce proliferation of KHYG-1 mCD16 cells (FIG. 32B), the PM-IL-15-CH34GS induced stronger proliferation of CTLL-2 cells compared to PM-IL-15-Cκ4GS (FIG. 32A) probably due to the differential expression of the IL-15R chains on the cell lines.

### Example 18

### Induction of Immune Cell Activation

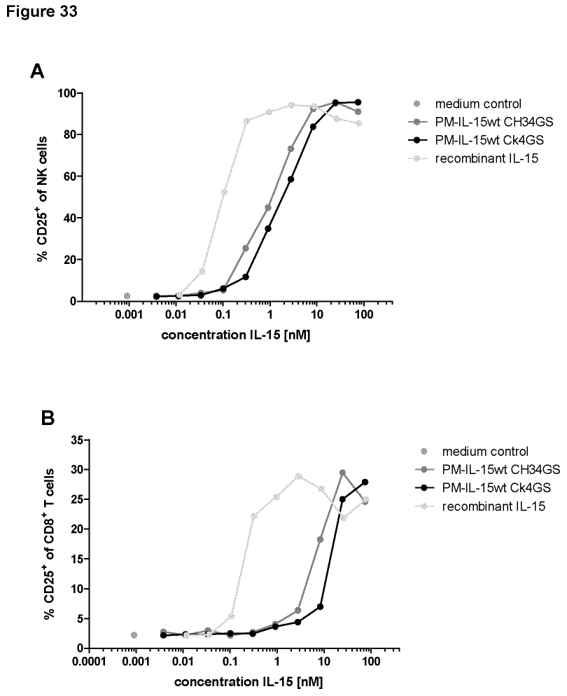

PM-IL-15 CH3 or Cκ fusion constructs were compared against recombinant IL-15 in their potency to activate primary immune cells. PBMC of a healthy donor were incubated for 5 days with the indicated molecules. Stimulated PBMC were stained with fluorescence labelled αCD3, αCD4, αCD8, αCD14, αCD25, αCD45, and αCD56 antibodies. Dead cells were excluded by addition of DAPI before analysis. As shown in FIG. 33, all tested molecules were able to induce expression of CD25 on NK cells (FIG. 33A) and CD8+ T cells (FIG. 33B). Again, when normalized to the molar concentration of IL-15 present in the assay recombinant IL-15 excelled in the activation of immune cells when compared to both PM-IL-15 constructs. Further, PM-IL-15-CH34GS had a slightly higher potency to induce the activation of immune effector cells compared to PM-IL-15-Cκ4GS.

### Example 19

### Induction of Anti-Tumor Cytotoxicity

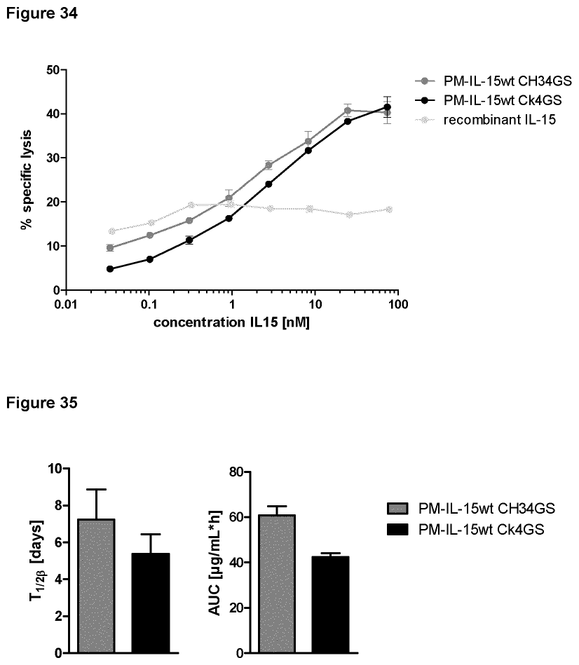

Next, it was investigated if similar differences between recombinant IL-15 and PM-IL-15 CH3 or Cκ fusion constructs could be observe in a tumor cell killing assay. For this purpose, TA-MUC1-positive CaOV-3 tumor cells were grown for 24 h in assay plates before addition of PM-IL-15-CH34GS, PM-IL-15-Cκ4GS or recombinant IL-15 and unstimulated PBMC at an effector to target cell ratio of 10:1. Tumor cell killing was assessed 24 h later by quantification of lactate dehydrogenase (LDH) released into cell supernatant (Cytotoxicity Detection Kit (LDH), Roche). Maximal release was achieved by incubation of target cells with triton-X-100 and antibody-independent cell death was measured in samples containing only target cells and PBMCs but no antibody. As shown in FIG. 34, both PM-IL-15-CH34GS and -Cκ4GS induced specific lysis of CaOV-3 tumor cells and the fusion CH3 fusion construct showed a higher activity compared to the Cκ fusion construct as observed before. Importantly, although recombinant IL-15 was superior in inducing activation of NK cells and CD8+ T cells (FIG. 33), this did not translate into effective immune cell mediated tumor cell lysis as observed for PM-IL-15-CH34GS and -Cκ4GS (FIG. 34, maximum lysis using recombinant IL-15 ˜20% compared to 40% using PM-IL-15).

### Example 20

### Influence of Construct Design on Pharmacokinetics In Vivo

To investigate if the fusion of IL-15 to the CH3 or Cκ domain of an antibody impacts its pharmacokinetic profile, C57BL/6 mice were injected i.v. with 2 mg/kg of the constructs (n=3). Serum was collected 5min, 6 h, 1 d, 2 d, 4 d, 7 d, and 11 d after injection and antibody titers were determined by ELISA. Maxisorp F96 ELISA plates were coated with 2 pg/ml mouse anti-human Igκ light chain antibody in 0.1 M carbonate buffer pH 9.6 overnight. Unspecific binding was blocked with 5% (v/v) bovine serum albumin (BSA) in PBS (BSA/PBS). Serum samples and the standard were diluted in 1% (v/v) BSA/5% (v/v) mouse serum/PBS and were detected using horseradish peroxidase (HRP)-conjugated goat anti-human (IgG, Fcγ fragment specific). For color development, TMB One Component HRP Microwell Substrate was added to the ELISA plate. The reaction was stopped with 1.25 M sulfuric acid and signals were measured at 450 nm and 620 nm using a Tecan Infinite F200 microplate reader.

As shown in FIG. 35, PM-IL-15-CH34GS exhibited a longer t_{1/2} and greater total exposition (AUC) in C57BL/6 mice compared to PM-IL-15-Cκ4GS. The same results were observed after s.c. injection of both constructs.

### Example 21

### Influence of Construct Design on Pharmacodynamics In vivo

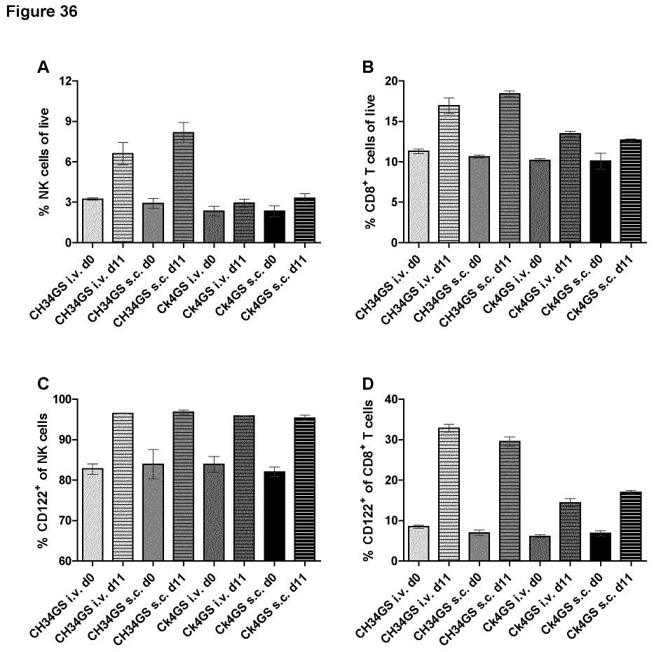

Next it was investigated if there are also differences in the pharmacodynamic profile of PM-IL-15-CH34GS and -Cκ4GS in vivo. Mice (C57BL/6, n=3) were injected i.v. and s.c. with either 2 mg/kg of PM-IL-15wt CH34GS or Ck4GS and were sacrificed on day 11 to collect blood. Immune cells from blood were characterized for phenotype and frequencies by flow cytometry using fluorescence labelled αCD3, αCD4, αCD8, αCD44, αCD45, αCD45R, αCD62L, αCD122, and αNK1.1 antibodies. Application of PM-IL-15-CH34GS lead to the expansion of NK cells (2 fold) after i.v. and s.c. injection while there was no significant effect on the NK cell compartment using PM-IL-15-Cκ4GS at this time point (FIG. 36A). However, injection of both constructs resulted in a similar up-regulation of CD122 on NK cells implying that also the Cκ4GS construct is able to engage NK cells (FIG. 36C). Analyzing CD8+ T cells, both constructs were able to increase the total frequency and CD122 expression of this subset but again PM-IL-15-CH34GS induced a stronger expansion (1.5 fold) and CD122 up-regulation than PM-IL-15-Cκ4GS (1.3 fold expansion) (FIG. 36B and D).

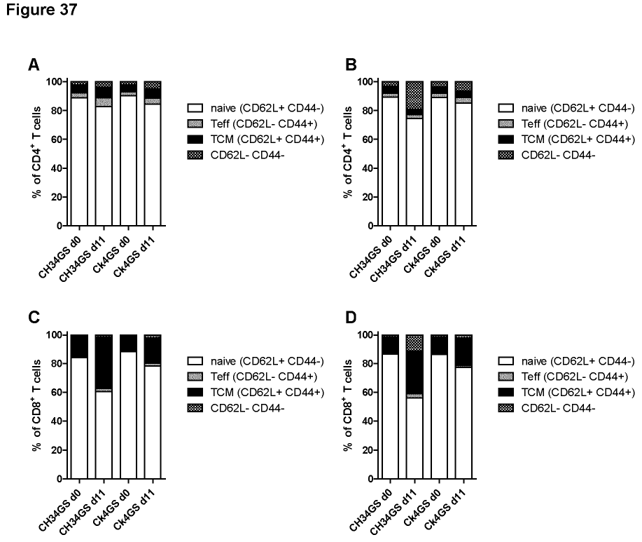

Within the CD4+ and CD8+ T cell population, treatment with PM-IL-15-CH34GS and PM-IL-15-Cκ4GS lead to a relative reduction of naïve cells and concurrently to an increase of cells with central memory (TCM) and effector (Teff) phenotype after i.v. (FIG. 37A and C) and s.c. injection (FIG. 37B and D). Further, PM-IL-15-CH34GS was superior in mobilizing CD4+ and CD8+ effector and memory T cells than the PM-IL-15-Cκ4GS construct independently of the used injection route.

### Example 22

### Therapeutic Efficacy in In Vivo Tumor Mmodel

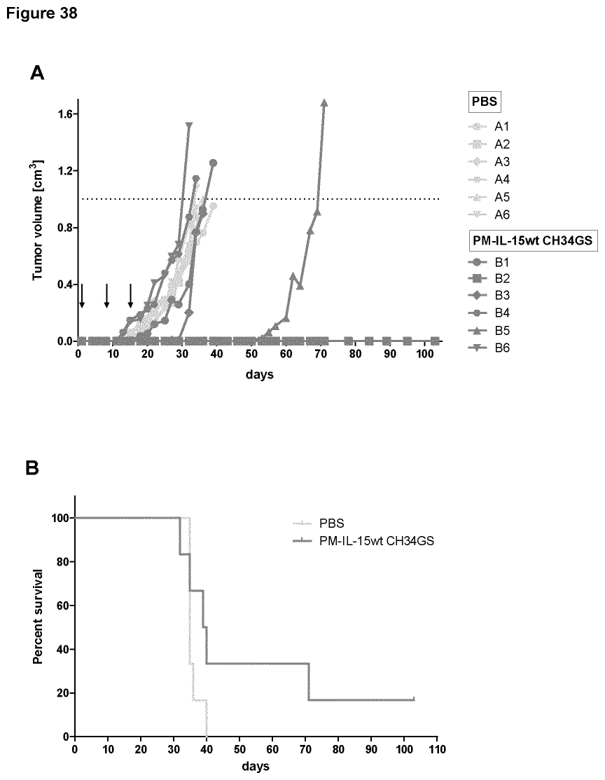

Next it was analyzed if a TA-MUC1 targeted IL-15 immunocytokine has the potential to improve the outcome of tumor bearing mice. Since the glyco-specific epitope TA-MUC1 is not found in mice, the mouse breast carcinoma cell line 4T1 was transfected with MUC1 and the TA-MUC1 expressing transfectant MUC1-4T1 was used as a tumor model for in vivo studies. Tumors derived of 4T1 cells are described to be highly immunosuppressive containing predominantly myeloid-derived suppressor cells. MUC1-4T1 cells were injected into the mammary fat pad (mfp) and treated on d1, d8, and d15 with 0.25 mg/kg PM-IL-15-CH34GS. Tumor volumes were monitored and mice were sacrificed when they reached a tumor burden of 1 cm³ according to state guidelines. FIG. 38A shows the tumor volume of PBS and PM-IL-15-CH34GS treated mice over time, the arrows indicate the dosing days. FIG. 38B displays the survival of the mice. Application of PM-IL-15-CH34GS lead to a tumor growth delay in 3 of 6 mice (FIG. 38A) in this highly suppressive model which was also reflected by a longer survival of PM-IL-15-CH34GS treated mice compared to the PBS group (FIG. 38B).

### Combination of PM-IL-15 with Other Therapeutics

### Example 23

### Activation of Immune Cells using PM-IL-15 in Combination with PM-CD3

The idea behind a therapy with a PM-IL-15 immunocytokine is that it not only activates immune cells by itself but additionally enhances ongoing immune responses by stimulating NK and T cells. To test this hypothesis, we combined a TA-MUC1 targeting T cell engager (PM-CD3; a bispecific antibody wherein scFv fragments against CD3 are fused to the C terminus of the heavy chains of the anti-TA-MUC1 antibody Pankomab) with PM-IL-15wt and PM-IL-15wt NA and analyzed T cell activation, T cell proliferation and cytotoxicity.

For the analysis of T cell activation, PBMC of a healthy donor were incubated with PM-IL-15-CH34GS in the absence or presence of a suboptimal concentration (0.4 μg/ml and 2 μg/ml, respectively) of PM-CD3 The activation of T cells was analyzed after 2 days by staining stimulated PBMC with fluorescence labelled αCD4, αCD8, αCD14, αCD19, αCD25, αCD45, αCD56, and αCD69 antibodies. Dead cells were excluded by addition of DAPI before analysis by flow cytometry.

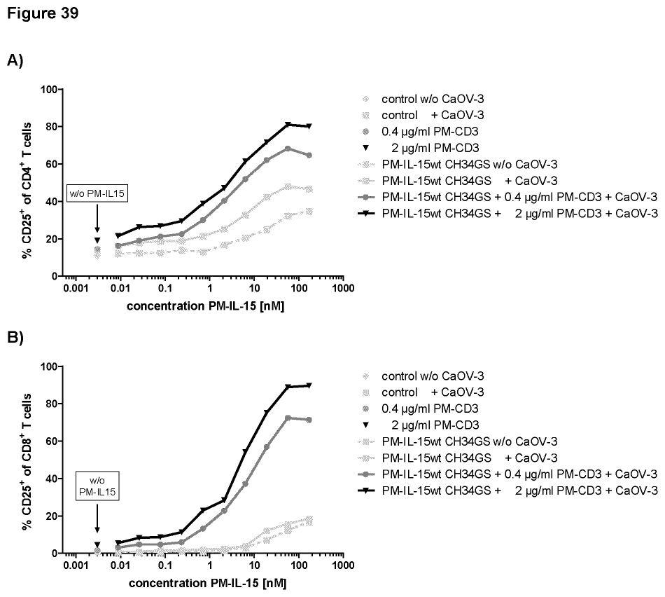

FIG. 39 shows that PM-CD3 as single therapy at suboptimal concentrations induced a slight expression of CD25 on CD4+ and CD8+ T cells. PM-IL-15wt induced a concentration-dependent increase of CD25 on both T cell subsets which was on CD4+ T cells further enhanced in the presence of CaOV-3 tumor cells. Interestingly, the combination of PM-IL-15wt with only 0.4 μg/ml (2 nM) PM-CD3 strongly enhanced the expression of CD25 on CD4+ and CD8+ T cells. Importantly, the observed effects were not only additive but highly synergistic and could be further enhanced by increasing the amount of PM-CD3 to 2 μg/ml (10 nM).

### Example 24

### Proliferation of Immune Cells using PM-IL-15 in Combination with PM-CD3

For the analysis of the effect of PM-IL-15 immunocytokines in PM-CD3 induced T cell proliferation, PBMC of a healthy donor were incubated with PM-CD3 in the absence or presence of 1 μg/ml PM-IL-15wt and PM-IL-15wt NA. CellTrace™ Violet (Thermo Fisher)-labelled PBMCs were incubated for 5 days with the indicated molecules and TA-MUC1 positive CaOV-3 tumor cells. Stimulated PBMCs were stained with fluorescence labelled αCD4, αCD8, αCD14, αCD19, αCD45 and αCD56 antibodies. Dead cells were excluded by staining with 7-AAD (Sigma-Aldrich) before analysis by flow cytometry.

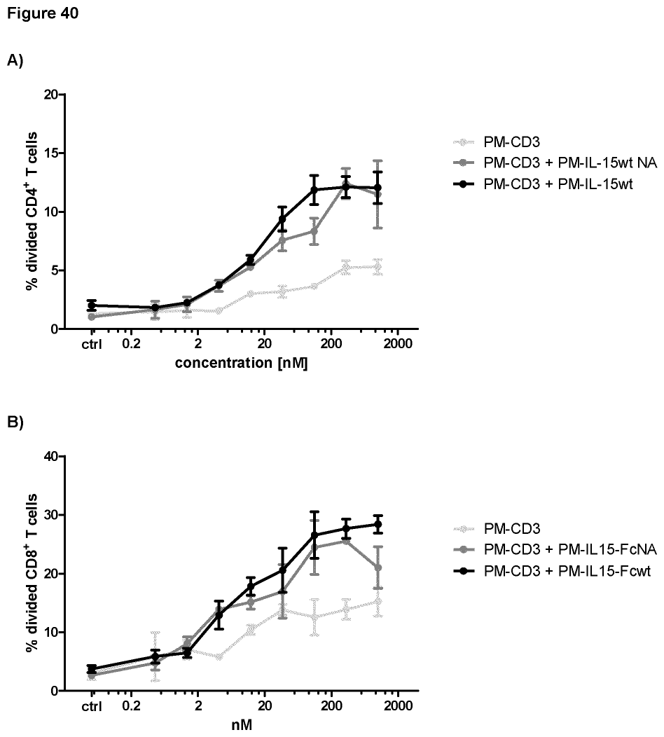

PM-CD3 alone was able to induce proliferation of CD4+ T cells (FIG. 40A) and CD8+ T cells (FIG. 40B) in the presence of CaOV-3 tumor cells. Surprisingly, while PM-IL-15wt and PM-IL-15wt NA were not able to induce proliferation of CD4+ and CD8+ T cells at this concentration by themselves, they strongly enhanced PM-CD3 mediated proliferation in a highly synergistic manner. The potency of PM-IL-15wt and PM-IL-15wt NA to stimulate PM-CD3 induced proliferation was comparable.

### Example 25

### Cytotoxicity of PM-IL-15 in Combination with PM-CD3

We also assessed the potential of combining PM-IL-15 immunocytokines and PM-CD3 in tumor cell killing. TA-MUC1-positive CaOV-3 tumor cells were grown for 24 h in assay plates before addition of unstimulated PBMC at an effector to target cell ratio of 10:1. PM-CD3 alone or in combination with indicated concentrations of PM-IL-15wt and PM-IL-15wt NA were added. Tumor cell killing was assessed 24 h later by quantification of lactate dehydrogenase (LDH) released into cell supernatant (Cytotoxicity Detection Kit (LDH), Roche). Maximal release was achieved by incubation of target cells with triton-X-100 and antibody-independent cell death was measured in samples containing only target cells and PBMCs but no antibody.

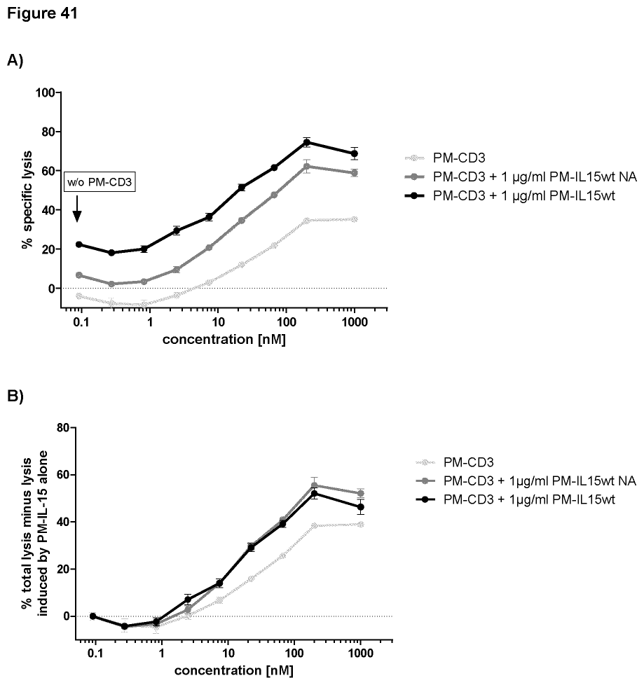

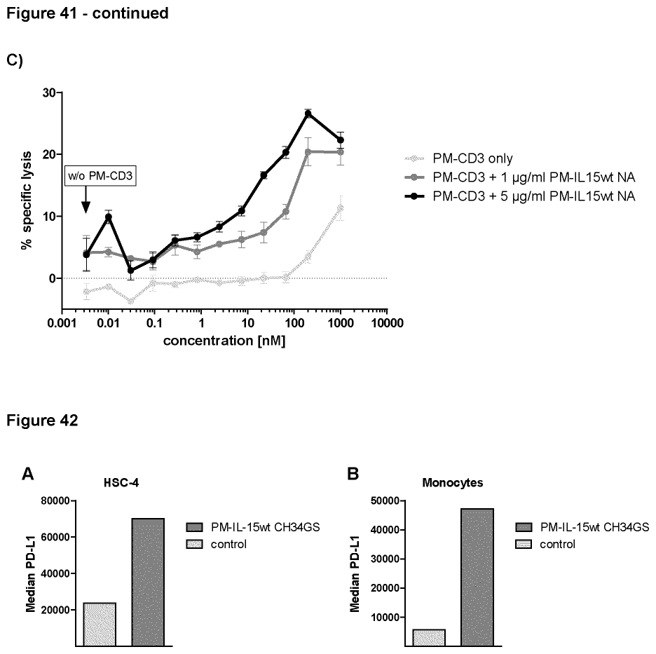

As single therapy, PM-CD3 and PM-IL-15 immunocytokines were able to induce a slight to moderate lysis of CaOV-3 tumor cells (FIG. 41). Surprisingly, addition of PM-IL-15wt or PM-IL-15wt NA enhanced the specific lysis of tumor cells again not only additively but synergistically (FIG. 41A and B). By adding only 1 μg/ml PM-IL-15wt to 200 nM PM-CD3 the specific lysis of CaOV-3 tumor cells was increased from 34% to 75%. The PM-IL-15wt NA construct was slightly less potent (maximum lysis 62%). FIG. 41C shows that the combination effect is also dependent on the concentration of the PM-IL-15 immunocytokines used. By increasing the concentration of PM-IL-15wt NA from 1 μg/ml to 5 μg/ml, we could further reduced EC50 values and increase the maximum lysis of tumor cells.

### Example 26

### Combination of PM-IL-15 Immunocytokine with a PD-L1 or PD-1 Targeting Therapy

As described before, the PM-IL-15 immunocytokine mediates the activation of immune cells by itself but is also able to enhance responses of NK and T cells induced by another drug (e.g. bispecific T cell engager). Checkpoint inhibitors targeting the PD-1/PD-L1 are widely used in the clinic and thus in vitro studies were performed to analyze if there is a rationale for combination of these agents with a TA-MUC1 targeting IL-15 immunocytokine. First it was analyzed if PD-L1 expression on tumor cells and monocytes is altered after incubation with PM-IL-15-CH34GS. TA-MUC1-positive HSC-4 tongue squamous carcinoma cells were grown for 24 h in assay plates before addition of freshly isolated PBMC at an effector to target cell ratio of 10:1. PM-IL-15-CH34GS (20 nM) or PBS were added and plates were cultured for 48 h. Tumor cells and PBMC were harvested and analyzed for the expression of PD-L1. As shown in FIG. 42, the addition of PM-IL-15-CH34GS significantly increased the expression of PD-L1 on HSC-4 tumor cells (FIG. 42A) and monocytes (FIG. 42B).

Next the potential of combining PM-IL-15 immunocytokines and the PD-L1 targeting antibody Avelumab (Bavencio®) in a tumor cell killing assay was investigated. TA-MUC1-positive HSC-4 tumor cells were grown for 24 h in assay plates before addition of unstimulated PBMC at an effector to target cell ratio of 10:1. PM-IL-15-CH34GS alone or in combination with indicated concentrations of Avelumab or hIgG1 as control were added. Tumor cell killing was assessed 24 h later by quantification of lactate dehydrogenase (LDH) released into cell supernatant as described before.

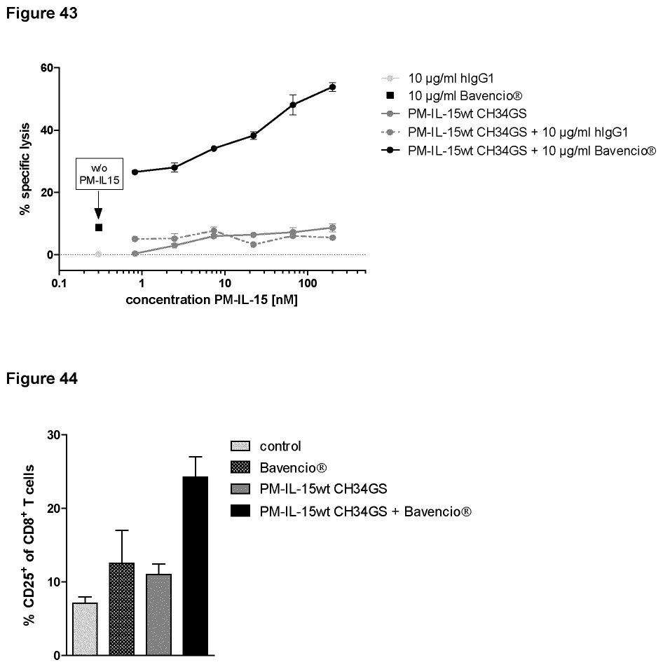

As single therapy, PM-IL-15-CH34GS and Avelumab were able to induce a slight lysis (˜9%) of HSC-4 tumor cells (FIG. 43). Surprisingly, the combination of PM-IL-15-CH34GS and Avelumab significantly increased the specific lysis of tumor cells not only additively but synergistically. A concentration of PM-IL-15-CH34GS of only 0.8 nM was sufficient to triplicate the tumor cells lysis induced by Avelumab alone (8.9% to 26.5%). This effect could be further enhanced by increasing the concentration of PM-IL-15-CH34GS (max. observed lysis 54%).

Another accepted method to investigate the ability of PD-1/PD-L1 checkpoint inhibitors to activate T cells in vitro is the allogeneic mixed lymphocyte reaction (MLR), where monocyte-derived dendritic cells (moDCs) and T cells from different donors are co-incubated to mimic immunosuppressive effects by the interaction of PD-L1 and PD-1. Monocytes were isolated from PBMC of a healthy donor and moDCs were generated by culturing the monocytes in medium supplemented with conditioned medium, GM-CSF, and IL-4. Seven days later, moDCs were harvested and cultured in 96-well plates together with allogeneic T cells at a ratio of 1:10 in the presence of 1 μg/ml of each test antibody. Cell were harvested 5 days later and analyzed by flow cytometry for the expression of CD25 on CD8+ T cells. Addition of either PM-IL-15-CH34GS and Avelumab lead to an increase of CD8 T cell activation determined by up-regulation of CD25 compared to the control (5.4% and 3.9% increase, respectively; FIG. 44). However, combination of PM-IL-15-CH34GS and Avelumab increased the expression of CD25 from 7.1% to 24.3% (17.2% increase) implying a synergism of both antibodies.

### Example 27

### Combination of PM-IL-15 Immunocytokine with Anti-EGFR Therapeutics

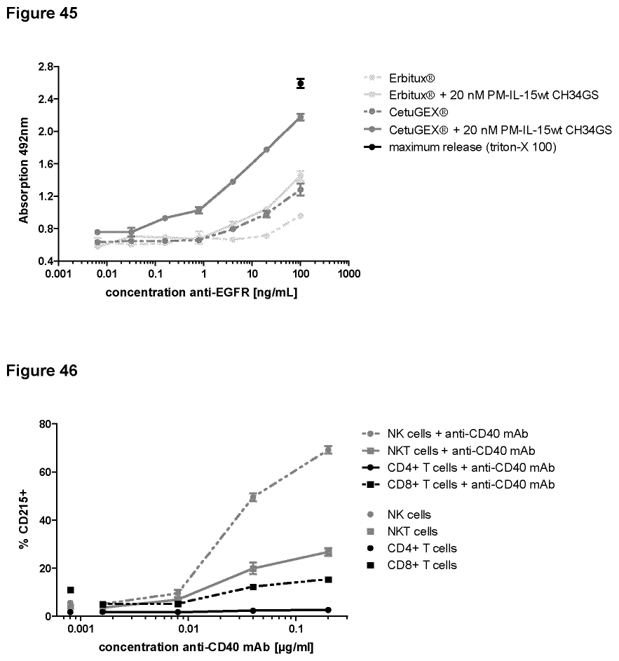

Next, it was investigated if EGFR-targeting therapeutics can be enhanced. Erbitux® (classical hIgG1 cetuximab) and CetuGEX® (tomuzotuximab, Glycotope's glyco-optimized second-generation cetuximab) were tested in combination with PM-IL-15-CH34GS in a tumor cell killing assay as described in Example 19. HSC-4 tumor cells that express similar amounts of TA-MUC1 and EGFR (internal analysis) were used as target cells. As shown in FIG. 45, CetuGEX® alone induced a concentration-dependent release of LDH that was higher than using Erbitux® alone. The addition of only 20 nM (3.5 μg/ml) PM-IL-15-CH34GS significantly increased the release of LDH in combination with Erbitux® and CetuGEX® at certain threshold concentrations (>0.1 ng/ml for CetuGEX® and >1 ng/ml for Erbitux®). This indicates that the PM-IL-15 immunocytokine has the potential to amplify responses to anti-EGFR treatment in a synergistic fashion.

### Example 28

### Up-Regulation of IL-15R Expression by a CD40 Agonist

Further interesting combination partners for PM-IL-15 immunocytokine are other immunostimulatory drugs like anti-CD40 antibodies. FIG. 46 shows the expression of IL-15Rα (CD215) on NK, NKT, CD4+, and CD8+ T cells analyzed by flow cytometry after incubation of PBMC for 3 days with increasing concentrations of a glyco-optimized anti-CD40 IgG1 (Glycotope GmbH) compared to untreated cells. Treatment with anti-CD40 resulted in the up-regulation of CD215 on NK, NKT, and CD8+ T cells but not on CD4+ T cells (FIG. 46). These data imply that cross-presentation of IL-15 could be improved in the presence of anti-CD40 mAb thereby augmenting the activity of an IL-15-based immunocytokine.

### Identification of the Deposited Biological Material

The cell lines DSM ACC 2806, DSM ACC 2807 and DSM ACC 2856 were deposited at the DSMZ—Deutsche Sammlung von Mikroorganismen and Zellkulturen GmbH, Inhoffenstraße 7B, 38124 Braunschweig (DE) by Glycotope GmbH, Robert-Rössle-Str. 10, 13125 Berlin (DE) on the dates indicated in the following table.

| Name of the | Accession | | Date of |
|---|---|---|---|
| Cell Line | Number | Depositor | Deposition |
| NM-H9D8 | DSM ACC 2806 | Glycotope | Sep. 15, 2006 |
| | | GmbH | |
| NM-H9D8-E6 | DSM ACC 2807 | Glycotope | Oct. 5, 2006 |
| | | GmbH | |
| NM-H9D8-E6Q12 | DSM ACC 2856 | Glycotope | Aug. 8, 2007 |
| | | GmbH | |

1. A fusion protein construct, comprising
(i) an antibody module specifically binding to MUC1, and
(ii) an IL-15 module.

2. The fusion protein construct according to claim 1, wherein the antibody module has one or more of the following characteristics
(a) comprises a heavy chain variable domain and a light chain variable domain;
(b) comprises two antibody heavy chains and two antibody light chains;
(c) is an IgG-type antibody module, in particular an IgG1-type antibody module;
(d) specifically binds to a TA-MUC1 epitope;
(e) comprises a set of CDR sequences with CDR-H1 having the amino acid sequence of SEQ ID NO: 1, CDR-H2 having the amino acid sequence of SEQ ID NO: 3, CDR-H3 having the amino acid sequence of SEQ ID NO: 5, CDR-L1 having the amino acid sequence of SEQ ID NO: 10, CDR-L2 having the amino acid sequence of SEQ ID NO: 12 and CDR-L3 having the amino acid sequence of SEQ ID NO: 14;
(f) comprises a set of CDR sequences with CDR-H1 having the amino acid sequence of SEQ ID NO: 1, CDR-H2 having the amino acid sequence of SEQ ID NO: 33, CDR-H3 having the amino acid sequence of SEQ ID NO: 5, CDR-L1 having the amino acid sequence of SEQ ID NO: 10, CDR-L2 having the amino acid sequence of SEQ ID NO: 12 and CDR-L3 having the amino acid sequence of SEQ ID NO: 14; and/or
(g) comprises at least one, in particular two, heavy chain variable region comprising the amino acid sequence of SEQ ID NO: 9 or 34 or an amino acid sequence which is at least 80% identical thereto, and at least one, in particular two, light chain variable region comprising the amino acid sequence of SEQ ID NO: 18 or an amino acid sequence which is at least 80% identical thereto.

3. The fusion protein construct according to claim 1, wherein the IL-15 module comprises human IL-15 or a fragment thereof.

4. The fusion protein construct according to claim 3, wherein the human IL-15 or the fragment thereof has one or more of the following characteristics
(a) specifically binds to an interleukin receptor comprising the IL-2 receptor β-chain, the common γ-chain and the IL-15 receptor α chain;
(b) comprises the sequence of SEQ ID NO: 21; and/or
(c) comprises a mutation decreasing receptor binding, such as 167E.

5. The fusion protein construct according to claim 3, wherein the IL-15 module further comprises human IL-15 receptor α chain or a fragment thereof which specifically binds to human IL-15.

6. The fusion protein construct according to claim 1, wherein the IL-15 module does not comprise comprises an IL-15 receptor α chain or a fragment thereof which specifically binds to IL-15.

7. The fusion protein construct according to claim 1, wherein the antibody module comprises an N-glycosylation site in any CH2 domain in the antibody heavy chains.

8. The fusion protein construct according to claim 1, comprising
(i) one antibody module which comprises two antibody heavy chains and two antibody light chains; and
(ii) two IL-15 modules, wherein one IL-15 module is fused to the C terminus of each antibody heavy chain via a peptide linker.

9. The fusion protein construct according to claim 1, comprising
(i) one antibody module which comprises two antibody heavy chains and two antibody light chains; and
(ii) two IL-15 modules, wherein one IL-15 module is fused to the C terminus of each antibody light chain via a peptide linker.

10. The fusion protein construct according to claim 1, comprising a further agent conjugated thereto.

11. A nucleic acid encoding the fusion protein construct according to claim 1.

12. An expression cassette or vector comprising the nucleic acid according to claim 11 and a promoter operatively connected with said nucleic acid.

13. A host cell comprising the nucleic acid according to claim 11.

14. A pharmaceutical composition comprising the fusion protein construct according to claim 1 and one or more further components selected from the group consisting of solvents, diluents, and excipients.

15. (canceled)

16. A method of treating, prognosing, diagnosing and/or monitoring of diseases associated with abnormal cell growth, infections, and diseases associated with reduced immune activity, using the fusion protein construct according to claim 1.

17. The method according to claim 16, wherein the disease associated with abnormal cell growth is cancer.

18. The method according to claim 16, wherein the fusion protein construct is used in combination with a further agent.

19. The method according to claim 18, wherein the further agent is selected from the group consisting of a bispecific antibody targeting MUC1 and CD3, an antibody against PD-L1, an antibody against EGFR, and an antibody against CD40.

20. The method of claim 17, wherein the cancer is selected from the group consisting of cancer of the breast, colon, stomach, liver, pancreas, kidney, blood, lung, endometrium, thyroid and ovary.

21. The method of claim 16, wherein the infection is a bacterial, viral, fungal or parasitic infection.

22. The method of claim 16, wherein the disease associated with reduced immune activity is an immunodeficiency.


SEQUENCE LISTING (34 sequences)

SEQ ID NO: 1 - protein, 5 aa, Artificial
  anti-TA-MUC1 CDR H1
NYWMN

SEQ ID NO: 2 - protein, 5 aa, Artificial
  anti-TA-MUC1 CDR H1
DAWMD

SEQ ID NO: 3 - protein, 19 aa, Artificial
  anti-TA-MUC1 CDR H2
EIRLKSNNYTTHYAESVKG

SEQ ID NO: 4 - protein, 19 aa, Artificial
  anti-TA-MUC1 CDR H2
EIRSKANNHATYYAESVKG

SEQ ID NO: 5 - protein, 6 aa, Artificial
  anti-TA-MUC1 CDR H3
HYYFDY

SEQ ID NO: 6 - protein, 7 aa, Artificial
  anti-TA-MUC1 CDR H3
GGYGFDY

SEQ ID NO: 7 - protein, 118 aa, Artificial Sequence
  anti-TA-MUC1 heavy chain variable region
EVKLVESGGGLVQPGGSMKLSCAASGFTFSDAWMDWVRQSPEKGLEWVAEIRSKANNHATYYAESVKGRFTLSRDVSKSSVYLQMNNLRAEDTGLYYCTRGGYGFDYWGQGTTLTVSS

SEQ ID NO: 8 - protein, 117 aa, Artificial Sequence
  anti-TA-MUC1 heavy chain variable region
EVKLVESGGGLVQPGGSMKLSCVASGFTFSNYWMNWVRQSPEKGLEWVAEIRLKSNNYTTHYAESVKGRFTISRDDSKSSVSLQMNNLRVEDTGLYYCTRHYYFDYWGQGTTLTVSS

SEQ ID NO: 9 - protein, 117 aa, Artificial
  anti-TA-MUC1 heavy chain variable region
EVQLVESGGGLVQPGGSMRLSCVASGFPFSNYWMNWVRQAPGKGLEWVGEIRLKSNNYTTHYAESVKGRFTISRDDSKNSLYLQMNSLKTEDTAVYYCTRHYYFDYWGQGTLVTVSS

SEQ ID NO: 10 - protein, 16 aa, Artificial
  anti-TA-MUC1 CDR L1
RSSKSLLHSNGITYFF

SEQ ID NO: 11 - protein, 16 aa, Artificial
  anti-TA-MUC1 CDR L1
RSSQSIVHSNGNTYLE

SEQ ID NO: 12 - protein, 7 aa, Artificial
  anti-TA-MUC1 CDR L2
QMSNLAS

SEQ ID NO: 13 - protein, 7 aa, Artificial
  anti-TA-MUC1 CDR L2
KVSNRFS

SEQ ID NO: 14 - protein, 9 aa, Artificial
  anti-TA-MUC1 CDR L3
AQNLELPPT

SEQ ID NO: 15 - protein, 9 aa, Artificial
  anti-TA-MUC1 CDR L3
FQGSHVPLT

SEQ ID NO: 16 - protein, 113 aa, Artificial Sequence
  anti-TA-MUC1 light chain variable region
DIVLTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPLTFGDGTKLELKR

SEQ ID NO: 17 - protein, 113 aa, Artificial Sequence
  anti-TA-MUC1 light chain variable region
DIVMTQAAFSNPVTLGTSASISCRSSKSLLHSNGITYFFWYLQKPGLSPQLLIYQMSNLASGVPDRFSSSGSGTDFTLRISRVEAEDVGVYYCAQNLELPPTFGGGTKLEIKR

SEQ ID NO: 18 - protein, 113 aa, Artificial
  anti-TA-MUC1 light chain vatiable region
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIKR

SEQ ID NO: 19 - protein, 4 aa, Artificial
  epitope
PDTR

SEQ ID NO: 20 - protein, 5 aa, Artificial
  epitope
PDTRP

SEQ ID NO: 21 - protein, 114 aa, Artificial Sequence
  IL-15
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 22 - protein, 65 aa, Artificial Sequence
  IL-15Ralpha sushi domain
ITCPPPMSVEHADIWVKSYSLYSRERYICNSGFKRKAGTSSLTECVLNKATNVAHWTTPSLKCIR

SEQ ID NO: 23 - protein, 6 aa, Artificial Sequence
  anti-CD3 CDR-H1
GYTFTR

SEQ ID NO: 24 - protein, 9 aa, Artificial Sequence
  anti-CD3 CDR-H2
YINPSRGYT

SEQ ID NO: 25 - protein, 10 aa, Artificial Sequence
  anti-CD3 CDR-H3
YYDDHYALDY

SEQ ID NO: 26 - protein, 119 aa, Artificial Sequence
  anti-CD3 heavy chain variable region
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYALDYWGQGTTLTVSS

SEQ ID NO: 27 - protein, 10 aa, Artificial Sequence
  anti-CD3 CDR-L1
RASSSVSYMN

SEQ ID NO: 28 - protein, 7 aa, Artificial Sequence
  anti-CD3 CDR-L2
DTSKVAS

SEQ ID NO: 29 - protein, 9 aa, Artificial Sequence
  anti-CD3 CDR-L3
QQWSSNPLT

SEQ ID NO: 30 - protein, 106 aa, Artificial Sequence
  anti-CD3 light chain variable region
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 31 - protein, 5 aa, Artificial Sequence
  linker repeat
GGGGS

SEQ ID NO: 32 - protein, 5 aa, Artificial Sequence
  linker repeat
PAPAP

SEQ ID NO: 33 - protein, 19 aa, Artificial Sequence
  anti-TA-MUC1 CDR H2
  VARIANT (8)..(8): Xaa is any amino acid
EIRLKSNXYTTHYAESVKG

SEQ ID NO: 34 - protein, 117 aa, Artificial Sequence
  anti-TA-MUC1 heavy chain variable region
  VARIANT (57)..(57): Xaa is any amino acid
EVQLVESGGGLVQPGGSMRLSCVASGFPFSNYWMNWVRQAPGKGLEWVGEIRLKSNXYTTHYAESVKGRFTISRDDSKNSLYLQMNSLKTEDTAVYYCTRHYYFDYWGQGTLVTVSS